(19) United States
(12) Patent Application Publication
(10) Pub. No.: US 2010/0196321 A1
(43) Pub. Date: Aug. 5, 2010

(54) COMPOUNDS

(75) Inventors: JOEL COOPER, Durham, NC (US); MAOSHENG DUAN, Durham, NC (US); RICHARD GRIMES, Durham, NC (US); WIESLAW KAZMIERSKI, Durham, NC (US); MATTHEW TALLANT, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GLAXOSMITHKLINE LLC, Philadelphia, PA (US)

(21) Appl. No.: 12/695,506

(22) Filed: Jan. 28, 2010

Related U.S. Application Data
(60) Provisional application No. 61/247,040, filed on Sep. 30, 2009.
(60) Provisional application No. 61/148,632, filed on Jan. 30, 2009.

(51) Int. Cl. A61K 31/496; C07D 487/08; A61K 31/4709; A61K 31/5377; A61K 31/541; A61K 38/21; A61K 31/7056; A61K 31/7088; A61P 31/12

(52) U.S. Cl. 424/85.7; 540/460; 514/210.18; 514/312; 514/233.2; 514/228.2; 514/253.07; 424/85.4; 514/43; 514/44 A

(57) ABSTRACT

The present invention features compounds of Formula (I) and (Ia), pharmaceutical compositions and use in the treatment of viral disease:

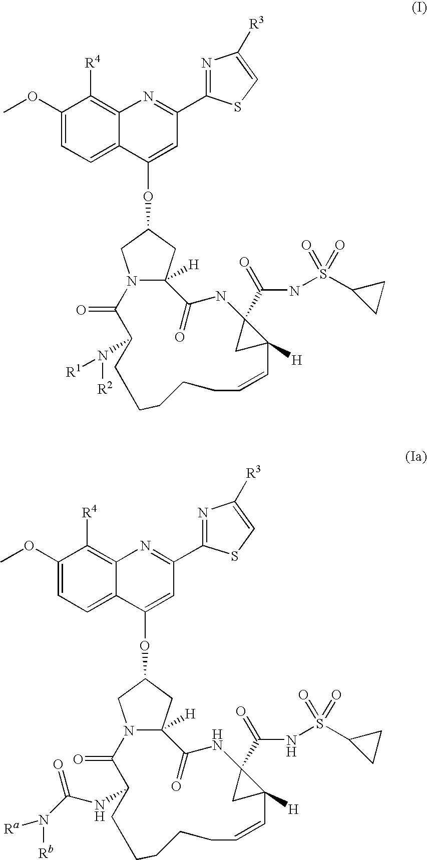

This application is filed pursuant to 35 U.S.C. §111(a) and claims priority from U.S. Provisional Application Ser. No. 61/148,632, filed Jan. 30, 2009, and U.S. Provisional Application Ser. No. 61/247,040, filed Sep. 30, 2009, the contents of both of which are hereby incorporated by reference herein.

### FIELD OF THE INVENTION

The present invention relates to compounds useful as anti-viral agents. Specifically, the present invention involves novel inhibitors of Hepatitis C Virus (HCV) replication.

### BACKGROUND OF THE INVENTION

Infection with HCV is a major cause of human liver disease throughout the world. Although only 30% of acute infections are symptomatic, greater than 85% of infected individuals develop chronic, persistent infection. In the US, an estimated 4.5 million Americans are chronically infected with HCV. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U.S.

Alpha-interferon (alone or in combination with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, (1997) Hepatology, 26 (suppl 1): 71S-77S).

The HCV NS3-4A protease is considered to be essential for replication of hepatitis C virus (Kolykhalov et al., (2000) Journal of Virology, 74, 2046-2051). Therefore, the use of protease inhibitors, in particular those that are selective HCV serine protease inhibitors have potential in treating HIV infections by inhibiting HCV replication.

### SUMMARY OF THE INVENTION

The present invention features macrocyclic compounds, pharmaceutical compositions comprising such compounds and use of the compounds in treating viral infection, especially HCV infection.

### DETAILED DESCRIPTION OF THE INVENTION

The present invention provides a compound of Formula (I):

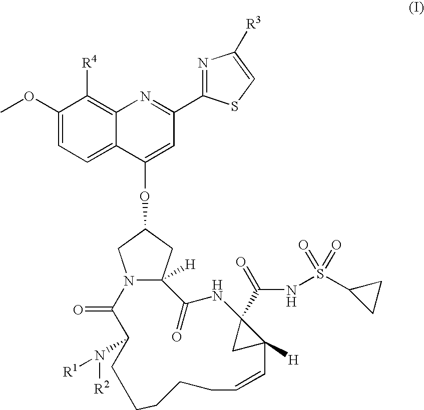

wherein:
R¹ is hydrogen, C₁-C₈ alkyl, haloalkyl, hydroxyalkyl;

### R² is C(O)XR^{a}R^{b};

X is N or O;
R^{a} and R^{b} are independently selected from the group consisting of hydrogen, C₁-C₈ alkyl, haloalkyl, hydroxyalkyl, C₁₋₆alkoxy, C₃-C₇ cycloalkyl, heteroaryl, or aryl;
or R^{a} and R^{b} together with the nitrogen to which they are attached form a four to seven membered heterocyclic ring,
   R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
   or a pharmaceutically acceptable salt thereof, provided that:
   if a) X is N and R^{a} is hydrogen, then R^{b} is not C₁-C₈ alkyl, haloalkyl or C₃-C₇ cycloalkyl; b) X is O, then R^{a} is absent, and R^{b} is not C₁-C₈ alkyl, haloalkyl or C₃-C₇ cycloalkyl.

The term “alkyl”, alone or in combination with any other term, refers to a straight-chain or branched-chain saturated aliphatic hydrocarbon radical containing the specified number of carbon atoms. Examples of alkyl radicals include, but are not limited to, methyl, ethyl, n-propyl, isopropyl, n-butyl, isobutyl, sec-butyl, tert-butyl, pentyl, isoamyl, n-hexyl and the like.

The term “cycloalkyl” refers to a saturated or partially saturated carbocyclic ring composed of 3-7 carbons in any chemically stable configuration. Examples of suitable carbocyclic groups include cyclopropyl, cyclobutyl, cyclopentyl, cyclohexyl, and cyclohexenyl.

The term “alkoxy” refers to an —O-alkyl group wherein alkyl is as defined herein. Examples of such groups include methoxy, ethoxy, propoxy, butoxy, pentoxy or hexoxy and the like. If specified herein, the alkoxy group may be substituted by one or more substituents.

The term, “halogen” or “halo” refers to a fluorine, chlorine, bromine or iodine atom.

References to “fluoro”, “chloro”, “bromo” or “iodo” should be construed accordingly.

The term “alicyclic” refers to a carbocyclic aliphatic ring containing 3 to 8 carbon atoms.

The term “alkylidene” refers to a divalent group formed from an alkane by removal of two hydrogen atoms from the same carbon atom, the free valencies of which are part of a double bond.

The term “alkylene” referres to a straight or branched chain divalent hydrocarbon radical, preferably having from one to twelve carbon atoms, unless otherwise defined. Examples of “alkylene” as used herein include, but are not limited to, methylene, ethylene, propylene, butylene, isobutylene and the like.

The term “aryl” alone or in combination with any other term, refers to a carbocyclic aromatic moiety (such as phenyl or naphthyl) containing the specified number of carbon atoms, preferably from 6-10 carbon atoms. “Aryl” includes carbocyclic aryl and biaryl groups. Examples of aryl radicals include, but are not limited to, phenyl, naphthyl, indenyl, azulenyl, fluorenyl, anthracenyl, phenanthrenyl, tetrahydronaphthyl, indanyl, phenanthridinyl and the like. Unless otherwise indicated, the term “aryl” also includes each possible positional isomer of an aromatic hydrocarbon radical, such as in 1-naphthyl, 2-naphthyl, 5-tetrahydronaphthyl, 6-tetrahydronaphthyl, 1-phenanthridinyl, 2-phenanthridinyl, 3-phenanthridinyl, 4-phenanthridinyl, 7-phenanthridinyl, 8-phenanthridinyl, 9-phenanthridinyl and 10-phenanthridinyl. Examples of aryl radicals include, but are not limited to, phenyl, naphthyl, indenyl, azulenyl, fluorenyl, anthracenyl, phenanthrenyl, tetrahydronaphthyl, indanyl, phenanthridinyl and the like.

As used herein, “heteroaryl” refers to a 5-, 6-, 8-, 9- or 10-membered monocyclic or bicyclic aromatic moiety comprising one to four heteroatoms selected from N, O and S. In one aspect, “heteroaryl” moieties are selected from pyridine, pyrazine, thiazole, thiophene, oxadiazole, oxazole, pyrimidine, pyridazine, triazole, tetrazole, benzodioxole, benzofuran, benzodioxin, indole, benzimidazole, benzofuran, indole, indazole, isoindole, benzothiophene, benzothiazole, benzoxazole, benzisoxazole, benzisothiazole, benzotriazole, furopyridine, furopyrimidine, furopyridazine, furopyrazine, furotriazine, pyrrolopyridine, pyrrolopyrimidine, pyrrolopyridazine, pyrrolopyrazine, pyrrolotriazine, thienopyridine, thienopyrimidine, thienopyridazine, thienopyrazine, thienotriazine, thiazolopyridine, thiazolopyrimidine, thiazolopyridazine, thiazolopyrazine, thiazolotriazine, oxazolopyridine, oxazolopyrimidine, oxazolopyridazine, oxazolopyrazine, oxazolotriazine, imidazopyridine, imidazopyrimidine, imidazopyridazine, imidazopyrazine, imidazotriazine, pyrazolopyridine, pyrazolopyrimidine, pyrazolopyridazine, pyrazolopyrazine, pyrazolotriazine, triazolopyridine, triazolopyrimidine, triazolopyridazine, triazolopyrazine, quinoline, naphthyridine, quinoxaline, quinazoline, isoquinoline, cinnoline, pyridopyridazine, pyridopyrimidine, pyridopyrazine, pyrazinopyrazine, pteridine, pyrazinopyridazine, pyrimidopyridazine, pyrimidopyrimidine, imidazothiazole and thiazolooxazole. All isomers of the above heteroaryl groups are within the scope of this invention. Each heteroaryl group may be attached at any ring carbon or may be attached through nitrogen when the nitrogen is part of a 5-membered ring.

The term “heterocycle,” “heterocyclic,” and “heterocyclyl” as used herein, refer to a 3- to 7-membered monocyclic heterocyclic ring or 8-to 11-membered bicyclic heterocyclic ring system any ring of which is either saturated or partially saturated and which may be optionally benzofused if monocyclic. Each heterocycle consists of one or more carbon atoms and from one to four heteroatoms selected from the group consisting of N, O and S, and wherein the nitrogen and sulfur heteroatoms may optionally be oxidized, and the nitrogen atom may optionally be quaternized, and including any bicyclic group in which any of the above-defined heterocyclic rings is fused to a benzene ring. The heterocyclic ring may be attached at any carbon or heteroatom, provided that the attachment results in the creation of a stable structure. Preferred heterocycles include 5-7 membered monocyclic heterocycles and 8-10 membered bicyclic heterocycles. When the heterocyclic ring has substituents, it is understood that the substituents may be attached to any atom in the ring, whether a heteroatom or a carbon atom, provided that a stable chemical structure results. Also included within the scope of the term “heterocycle, “heterocyclic” or “heterocyclyl” is a group in which a non-aromatic heteroatom-containing ring is fused to one or more aromatic rings, such as in an indolinyl, chromanyl, phenanthridinyl or tetrahydro-quinolinyl, where the radical or point of attachment is on the non-aromatic heteroatom-containing ring. Unless otherwise indicated, the term “heterocycle, “heterocyclic” or “heterocyclyl” also included each possible positional isomer of a heterocyclic radical, such as in 1-indolinyl, 2-indolinyl, 3-indolinyl. Examples of heterocycles include imidazolyl, imidazolinoyl, imidazolidinyl, quinolyl, isoquinolyl, indolyl, indazolyl, indazolinolyl, perhydropyridazyl, pyridazyl, pyridyl, pyrrolyl, pyrrolinyl, pyrrolidinyl, pyrazolyl, pyrazinyl, quinoxolyl, piperidinyl, pyranyl, pyrazolinyl, piperazinyl, pyrimidinyl, pyridazinyl, morpholinyl, thiamorpholinyl, furyl, thienyl, triazolyl, thiazolyl, carbolinyl, tetrazolyl, thiazolidinyl, benzofuranoyl, thiamorpholinyl sulfone, oxazolyl, oxadiazolyl, benzoxazolyl, oxopiperidinyl, oxopyrrolidinyl, oxoazepinyl, azepinyl, isoxozolyl, isothiazolyl, furazanyl, tetrahydropyranyl, tetrahydrofuranyl, thiazolyl, thiadiazoyl, dioxolyl, dioxinyl, oxathiolyl, benzodioxolyl, dithiolyl, thiophenyl, tetrahydrothiophenyl, sulfolanyl, dioxanyl, dioxolanyl, tetahydrofurodihydrofuranyl, tetrahydropyranodihydrofuranyl, dihydropyranyl, tetradyrofurofuranyl and tetrahydropyranofuranyl.

The term “heteroatom” means nitrogen, oxygen, or sulfur and includes any oxidized form of nitrogen, such as N(O) {N⁺—O⁻} and sulfur such as S(O) and S(O)₂, and the quaternized form of any basic nitrogen.

As used herein, the term “pharmaceutically acceptable” used in relation to an ingredient (such as an active ingredient, a salt thereof or an excipient) which may be included in a pharmaceutical formulation for administration to a patient, refers to that ingredient being acceptable in the sense of being compatible with any other ingredients present in the pharmaceutical formulation and not being deleterious to the recipient thereof.

The term “treatment” as used herein refers to the alleviation of symptoms of a particular disorder in a patient, or the improvement of an ascertainable measurement associated with a particular disorder, and may include the suppression of symptom recurrence in an asymptomatic patient such as a patient in whom a viral infection has become latent. Treatment may include prophylaxis which refers to preventing a disease or condition or preventing the occurrence of symptoms of such a disease or condition, in a patient. As used herein, the term “patient” refers to a mammal, including a human.

As used herein, the term “subject” refers to a patient, animal or a biological sample. The term “biological sample”, as used herein, includes, without limitation, cell cultures or extracts thereof; preparations of an enzyme suitable for in vitro assay; biopsied material obtained from a mammal or extracts thereof; and blood, saliva, urine, feces, semen, tears, or other body fluids or extracts thereof.

The present invention also features a compound of formula (I) as described above wherein

R¹ is hydrogen, C₁-C₈ alkyl, haloalkyl, hydroxyalkyl;

### R² is C(O)XR^{a}R^{b};

X is N or O;
R^{a} and R^{b} are independently selected from the group consisting of hydroxyalkyl, C₁₋₆alkoxy, heteroaryl, and aryl, or R^{a} and R^{b} together with the nitrogen to which they are attached form a four to seven membered heterocyclic ring, and wherein if X is O, then R^{a} is absent;
   R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
   or a pharmaceutically acceptable salt thereof.

The present invention also features a compound of formula (I) as described above wherein

R¹ is hydrogen, C₁-C₈ alkyl, haloalkyl, hydroxyalkyl,;

### R² is C(O)XR^{a}R^{b};

X is N or O;
R^{a} and R^{b} together form a four to seven membered heterocyclic ring;
   R³ and R⁴ are independently selected from the group consisting of C₁-C₈ alkyl, C₁-C₈ hydroxyalkyl, halogen, haloalkyl, C₁₋₈alkoxy, C₃-C₇ cycloalkyl, heteroaryl, and aryl;
   or a pharmaceutically acceptable salt thereof.

The present invention features a compound of formula (I) as described above wherein:

R¹ is hydrogen, C₁-C₈ alkyl, haloalkyl, hydroxyalkyl;

### R² is C(O)XR^{a}R^{b};

X is N or O;
R^{a} is hydrogen and R^{b} is selected from the group consisting of hydroxyalkyl, heteroaryl, and aryl, or R^{a} and R^{b} together form a four to seven membered heterocyclic ring;
   R³ and R⁴ are independently selected from the group consisting of C₁-C₈ alkyl, C₁-C₈ hydroxyalkyl, halogen, haloalkyl, C₁₋₈alkoxy, C₃-C₇ cycloalkyl, heteroaryl, and aryl;
   or a pharmaceutically acceptable salt thereof.

The present invention features a compound of formula (I) as described above wherein X is O.

The present invention features a compound of formula (I) as described above wherein R^{a} and R^{b} together with the nitrogen atom to which they are attached form a four to seven membered heterocyclic ring.

The present invention features a compound of formula (I) as described above wherein R¹ is hydrogen.

The present invention also features a compound of formula (Ia)

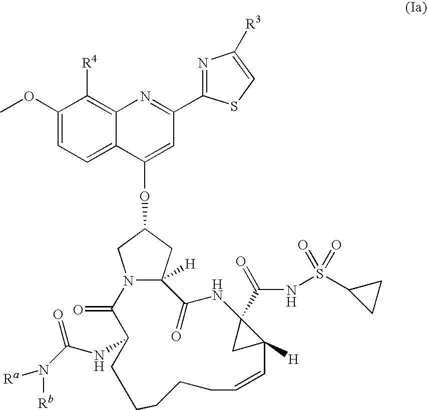

wherein:
R^{a} and R^{b} are independently selected from the group consisting of hydrogen, C₁-C₈ alkyl, haloalkyl, hydroxyalkyl, C₁₋₆alkoxy, C₃-C₇ cycloalkyl, heteroaryl, or aryl;
   or R^{a} and R^{b} together with the nitrogen to which they are attached form a four to seven membered heterocyclic ring;
      R³ and R⁴ are independently selected from the group consisting of C₁-C₈ alkyl, C₁-C₈ hydroxyalkyl, halogen, haloalkyl, C₁₋₈alkoxy, C₃-C₇ cycloalkyl, heteroaryl, and aryl;
      or a pharmaceutically acceptable salt thereof, provided that:
      when R^{a} is hydrogen, then R^{b} is not C₁-C₈ alkyl, haloalkyl or C₃-C₇ cycloalkyl.

The present invention also features a compound of formula (Ia) wherein:
R^{a} and R^{b} are independently selected from the group consisting of C₁-C₈ alkyl, haloalkyl, hydroxyalkyl, C₁₋₆alkoxy, C₃-C₇ cycloalkyl, heteroaryl, or aryl;
   or R^{a} and R^{b} together with the nitrogen to which they are attached form a four to seven membered heterocyclic ring;
      R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
      or a pharmaceutically acceptable salt thereof.

The present invention features a compound of formula (Ia) as described above wherein:

R^{a} and R^{b} are independently selected from the group consisting of hydroxyalkyl, Cᵢ₋ₑalkoxy, heteroaryl, and aryl, or R^{a} and R^{b} together with the nitrogen to which they are attached form a four to seven membered heterocyclic ring;
R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
or a pharmaceutically acceptable salt thereof.

The present invention features a compound of formula (Ia) as described above wherein:

R^{a} is hydrogen and R^{b} is selected from the group consisting of hydroxyalkyl, C₁₋₆alkoxy, heteroaryl, and aryl, or R^{a} and R^{b} together with the nitrogen to which they are attached form a four to seven membered heterocyclic ring;
R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
or a pharmaceutically acceptable salt thereof.

The present invention features a compound of formula (I) or (Ia) wherein R^{a} and R^{b} are both C₁-C₈ alkyl, haloalkyl, hydroxyalkyl, C₃-C₆ cycloalkyl, heteroaryl, and aryl; or a pharmaceutically acceptable salt thereof.

The present invention features a compound of formula (Ia) as described above wherein R^{a} and R^{b} together with the nitrogen atom to which they are attached form a four to seven membered heterocyclic ring.

The present invention features a compound of formula (Ia) wherein R³ is C₁-C₈ alkyl or C₃-C₆ cycloalkyl; R⁴ is C₁-C₈ alkyl or halogen; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

The present invention features a compound of formula (Ia) wherein R³ is cyclopropyl or isopropyl; R⁴ is C₁-C₈ alkyl; R^{a} is hydrogen or C₁-C₈ alkyl, and R^{b} is selected from the group consisting of hydrogen, C₁-C₈ alkyl, C₃-C₇ cycloalkyl, heteroaryl, aryl, cycloalkylalkyl and alkoxyalkyl.

The present invention features a compound of formula (Ia) wherein R³ is C₁-C₈ alkyl or C₃-C₆ cycloalkyl; R⁴ is C₁-C₈ alkyl or halogen; and R^{a} and R^{b} together with the nitrogen to which they are attached form a four to eight membered heterocyclic ring optionally substituted with one or more substituents selected from the group consisting of C₁-C₈ alkyl, hydroxyalkyl, C₃-C₇ cycloalkyl, alkylidene, hydroxy , halogen, oxo, aryl, heterocyclyl, R^{c}C(O)NH₂ and R^{c} C(O)OH wherein R^{o} is alklylene.

The present invention features a compound of formula (Ia) wherein R³ is isopropyl or cyclopropyl; and R^{a} and R^{b} together with the nitrogen to which they are attached form a four to eight membered heterocyclic ring optionally substituted with one or more substituents selected from the group consisting of C₁-C₈ alkyl, hydroxyalkyl, C₃-C₇ cycloalkyl, alkylidene, hydroxy , halogen, oxo, aryl, heterocyclyl, R^{c}C(O)NH₂ and R^{c}C(O)OH wherein R^{c} is alklylene.

The present invention features a compound of formula (Ia) as described above wherein the four to eight membered heterocyclic ring is selected from the group consisting of morpholinyl, a thiomorpholinyl, a piperidinyl, a piperazinyl, a pyrrolidinyl and an azetidinyl heterocyclic ring.

The present invention features a compound of formula (Ia) wherein R³ is C₁-C₈ alkyl or C₃-C₆ cycloalkyl; R⁴ is halogen; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

The present invention features a compound of formula (Ia) wherein R³ is C₁-C₈ alkyl or C₃-C₆ cycloalkyl; R⁴ is chloro; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

The present invention features a compound of formula (Ia) wherein R³ is cyclopropyl, and R⁴ is C₁-C₈ alkyl or halogen; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

The present invention features a compound of formula (Ia) wherein R³ is isopropyl, and R⁴ is C₁-C₈ alkyl or halogen; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

The present invention features a compound of formula (Ia) wherein R³ is C₁-C₈ alkyl or C₃-C₆ cycloalkyl; R⁴ is methyl; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

The present invention features a compound of formula (Ia) wherein R³ is cyclopropyl or isopropyl, R⁴ is C₁-C₆ alkyl or halogen; R^{a} is hydrogen and R^{b} is hydrogen.

The present invention features a compound of formula (Ia) wherein R³ is C₁-C₈ alkyl or C₃-C₆ cycloalkyl; R⁴ is methyl; R^{a} is C₁-C₈ alkyl; and R^{b} is selected from the group consisting of C₁-C₈ alkyl, C₃-C₇ cycloalkyl, heteroaryl, aryl, cycloalkylalkyl and alkoxyalkyl.

The present invention features a compound of formula (Ia) wherein R³ is cyclopropyl or isopropyl; R⁴ is halogen or C₁-C₈ alkyl; R^{a} is methyl, ethyl, propyl or isopropyl, and R^{b} is selected from the group consisting of hydrogen, C₁-C₈ alkyl, C₃-C₇ cycloalkyl, heteroaryl, aryl, cycloalkylalkyl and alkoxyalkyl.

The present invention features a compound of formula (Ia) wherein R³ is cyclopropyl or isopropyl; R⁴ is halogen or C₁-C₈ alkyl; R^{a} is methyl, ethyl, propyl, or isopropyl, and R^{b} is methyl, ethyl, propyl, and isopropyl.

The present invention features a compound of formula (Ia) as described above wherein the four to eight membered heterocyclic ring is selected from the group consisting of a morpholinyl, a thiomorpholinyl, a piperidinyl, a piperazinyl, a pyrrolidinyl and an azetidinyl.

The present invention features a compound of formula (Ia) wherein R³ is isopropyl, and R⁴ is C₁-C₈ alkyl or halogen; R^{a} is C₁-C₈ alkyl; and R^{b} is selected from the group consisting of C₁-C₈ alkyl, C₃-C₇ cycloalkyl, heteroaryl, aryl, cycloalkylalkyl and alkoxyalkyl.

The present invention features a compound of formula (Ia) wherein R³ is isopropyl, and R⁴ is methyl; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

The present invention features a compound of formula (Ia) wherein R³ is cyclopropyl or isopropyl; R⁴ is methyl; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

The present invention features a compound of formula (Ia) wherein R³ is isopropyl; R⁴ is methyl; R^{a} is methyl or ethyl or propyl or isopropyl, and R^{b} is methyl, ethyl, propyl, or isopropyl.

The present invention features a compound of formula (Ia) wherein R³ is cyclopropyl or isopropyl, and R⁴ is methyl; R^{a} is C₁-C₈ alkyl, and R^{b} is selected from the group consisting of C₁-C₈ alkyl, C₃-C₇ cycloalkyl, heteroaryl, aryl, cycloalkylalkyl and alkoxyalkyl.

The present invention features a compound of formula (Ia) wherein R³ is isopropyl, and R⁴ is methyl; R^{a} is C₁-C₈ alkyl, and R^{b} is selected from the group consisting of C₁-C₈ alkyl, C₃-C₇ cycloalkyl, heteroaryl, aryl, cycloalkylalkyl and alkoxyalkyl.

The present invention features a compound of formula (Ia) wherein R³ is isopropyl, and R⁴ is methyl; R^{a} and R^{b} together with the nitrogen to which they are attached form a four to eight membered heterocyclic ring.

The present invention features a compound of formula (Ia) wherein R³ is isopropyl; R⁴ is methyl; R^{a} and R^{b} together with the nitrogen to which they are attached form a four to eight membered heterocyclic ring selected from a morpholinyl, a thiomorpholinyl, a hexahydrocyclopenta[c]pyrrol-2(1H)-yl, a piperidinyl, a piperazinyl, a pyrrolidinyl and an azetidinyl heterocyclic ring.

The present invention features a compound of formula (Ia) wherein R³ is isopropyl, and R⁴ is methyl; R^{a} and R^{b} together with the nitrogen to which they are attached form a four to eight membered heterocyclic ring selected from a piperidinyl, a piperazinyl, a pyrrolidinyl and an azetidinyl heterocyclic ring.

The present invention features a compound of formula (Ia) wherein R³ is isopropyl or cyclopropyl, and R⁴ is halogen or C₁-C₈ alkyl; R^{a} and R^{b} together with the nitrogen to which they are attached form a four to eight membered heterocyclic ring selected from a piperidinyl, a piperazinyl, a pyrrolidinyl and an azetidinyl heterocyclic ring.

The present invention features a compound of formula (Ia) wherein R³ is isopropyl or cyclopropyl, and R⁴ is halogen or C₁-C₈ alkyl; R^{a} and R^{b} together with the nitrogen to which they are attached form a piperidinyl heterocyclic ring.

The present invention features a compound of formula (Ia) wherein R³ is isopropyl, R⁴ is halogen or C₁-C₈ alkyl; R^{a} and R^{b} together with the nitrogen to which they are attached form a piperidinyl heterocyclic ring.

The present invention features a compound of formula (Ia) wherein R³ is isopropyl or cyclopropyl, and R⁴ is methyl; R^{a} and R^{b} together with the nitrogen to which they are attached form a heterocyclic piperidinyl ring.

The present invention features a compound of formula (Ia) wherein R³ is isopropyl; R⁴ is halogen or C₁-C₈ alkyl; and R^{a} and R^{b} together with the nitrogen to which they are attached form a four to eight membered heterocyclic ring optionally substituted with one or more substituents selected from the group consisting of C₁-C₈ alkyl, hydroxyalkyl, C₃-C₇ cycloalkyl, alkylidene, hydroxy , halogen, oxo, aryl, heterocyclyl, R^{c}C(O)NH₂ and R^{c}C(O)OH wherein R^{e} is alkylene.

The present invention features a compound of formula (Ia) wherein R³ is isopropyl or cyclopropyl; R⁴ is methyl; and R^{a} and R^{b} together with the nitrogen to which they are attached form a four to eight membered heterocyclic ring optionally substituted with one or more substituents selected from the group consisting of C₁-C₈ alkyl, hydroxyalkyl, C₃-C₇ cycloalkyl, alkylidene, hydroxy , halogen, oxo, aryl, heterocyclyl, R^{c}C(O)NH₂ and R^{c}C(O)OH wherein R^{e} is alkylene.

The present invention features a compound of formula (Ia) wherein R³ is isopropyl; R⁴ is methyl; and R^{a} and R^{b} together with the nitrogen to which they are attached form a four to eight membered heterocyclic ring optionally substituted with one or more substituents selected from the group consisting of C₁-C₈ alkyl, hydroxyalkyl, C₃-C₇ cycloalkyl, alkylidene, hydroxy , halogen, oxo, aryl, heterocyclyl, R^{c}C(O)NH₂ and R^{c}C(O)OH wherein R^{e} is alkylene.

A combination of substituents or variables is permissible only if such combination results in a stable or chemically feasible compound.

It will be appreciated that the compounds of Formula (I) and (la) or salts thereof may contain one or more asymmetric carbon atoms and may exist as racemates, racemic mixtures and as individual enantiomers or diastereomers. All such isomeric forms are included within the present invention, including mixtures thereof. Although the specific compounds exemplified herein may be depicted in a particular stereochemical configuration, compounds having either the opposite stereochemistry at any given chiral center or mixtures thereof are also envisioned. Racemic compounds may either be separated using preparative HPLC and a column with a chiral stationary phase or resolved to yield individual enantiomers utilising methods known to those skilled in the art. In addition, chiral intermediate compounds may be resolved and used to prepare chiral compounds of Formula (I) or (Ia) or salts thereof.

The compounds of Formula (I) or (Ia) may exist in different tautomeric forms, i.e. one or more tautomeric forms. All tautomers, and mixtures thereof, are contemplated to be within the scope of the present invention. For example, a claim to 2-hydroxyquinolinyl would also cover its tautomeric form, α-quinolinonyl (2-quinolinonyl).

The present invention features a compound selected from the group consisting of:
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-{[(phenylamino)carbonyl]amino}-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-6-{[(diethylamino)carbonyl]amino}-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-6-[(aminocarbonyl)amino]-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS,Z)-2-(8-chloro-2-(4-cyclopropylthiazol-2-yl)-7-methoxyquinolin-4-yloxy)-N-(cyclopropylsulfonyl)-6-(3,3-diethylureido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
1,1-dimethylethyl ((2R,6S,13aS,14aR,16aS)-14a-{[(cyclopropylsulfonyl)amino]carbonyl}-2-{[8-methyl-2-[4-(1-methylethyl)-1,3-thiazol-2-yl]-7-(methyloxy)-4-quinolinyl]oxy}-5,16-dioxo-1,2,3,5,6,7,8,9,10,11,13a,14,14a,15,16,16a-hexadecahydrocyclopropa[e]pyrrolo[1,2-a][1,4]diazacyclopentadecin-6-yl)carbamate; and
   pharmaceutically acceptable salts thereof.

The present invention features a compound selected from the group consisting of:
(2R,6S,13aS,14aR,16aS)-N-(cyclopropylsulfonyl)-6-({[(2R,6S)-2,6-dimethyl-4-morpholinyl]carbonyl}amino)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-6-{[(1,1-dioxido-4-thiomorpholinyl)carbonyl]amino}-2-[{8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-6-{[(4-cyclopentyl-1-piperazinyl)carbonyl]amino}-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-6-[(4-morpholinylcarbonyl)amino]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-[(1-pyrrolidinylcarbonyl)amino]-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-[(1-piperidinylcarbonyl)amino]-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-6-[(1-azetidinylcarbonyl)amino]-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-{[(4-phenyl-1-piperidinyl)carbonyl]amino}-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-({[4-(1-pyrrolidinyl)-1-piperidinyl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-6-{[(4-hydroxy-1-piperidinyl)carbonyl]amino}-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-6-({[4-(2-amino-2-oxoethyl)-1-piperidinyl]carbonyl}amino)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;.
(2R,6S,13aS,14aR,16aS)-N-(cyclopropylsulfonyl)-6-[(hexahydrocyclopenta[c]pyrrol-2(1H)-ylcarbonyl)amino]-2-{[8-methyl-2-[4-(1-methylethyl)-1 , 3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-6-({[4-(hydroxymethyl)-1-piperidinyl]carbonyl}amino)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S ,13aS,14aR,16aS ,Z)-N-(cyclopropylsulfonyl)-2-(2-(4-cyclopropylthiazol-2-yl)-7-methoxy-8-methylquinolin-4-yloxy)-6-(3,3-diethylureido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-2-(8-chloro-2-(4-cyclopropylthiazol-2-yl)-7-methoxyquinolin-4-yloxy)-N-(cyclopropylsulfonyl)-6-(4-methylenepiperidine-1-carboxamido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-N-(cyclopropylsulfonyl)-2-(2-(4-cyclopropylthiazol-2-yl)-7-methoxy-8-methylquinolin-4-yloxy)-5,16-dioxo-6-(pyrrolidine-1-carboxamido)-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-N-(cyclopropylsulfonyl)-2-(2-(4-cyclopropylthiazol-2-yl)-7-methoxy-8-methylquinolin-4-yloxy)-5,16-dioxo-6-(piperidine-1-carboxamido)-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-6-(azetidine-1-carboxamido)-N-(cyclopropylsulfonyl)-2-(2-(4-cyclopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S ,13aS,14aR,16aS,Z)-N-(cyclopropylsulfonyl)-2-(2-(4-cyclopropylthiazol-2-yl)-7-methoxy-8-methylquinolin-4-yloxy)-6-(3,3-difluoroazetidine-1-carboxamido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S ,13aS,14aR,16aS ,Z)-N-(cyclopropylsulfonyl)-2-(2-(4-cyclopropylthiazol-2-yl)-7-methoxy-8-methylquinolin-4-yloxy)-6-(3,3-dimethylpyrrolidine-1-carboxamido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-N-(cyclopropylsulfonyl)-6-(3,3-difluoroazetidine-1-carboxamido)-2-(2-(4-isopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S ,13aS,14aR,16aS,Z)-N-(cyclopropylsulfonyl)-6-(3,3-dimethylpyrrolidine-1-carboxamido)-2-(2-(4-isopropylthiazol-2-yl)-7-methoxy-8-methylquinolin-4-yloxy)-5,16-dioxo-1,2,3,5,6,7,8,9,10,11 ,13a,14,14a,15,16,16a-hexadecahydrocyclopropa[e]pyrrolo[1,2-a][1,4]diazacyclopentadecine-14a-carboxamide;
   and pharmaceutically acceptable salts thereof.

The compounds of the present invention may be in the form of their free base or pharmaceutically acceptable salts, pharmaceutically acceptable solvates (including pharmaceutically acceptable solvates of salts) or pharmaceutically acceptable esters thereof.

The present invention also provides a pharmaceutically acceptable salt of a compound of Formula (I) or (Ia).

Pharmaceutically acceptable salts of the compounds according to the invention include those derived from pharmaceutically acceptable inorganic and organic acids and bases. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g. sodium), alkaline earth metal (e.g., magnesium), ammonium, NW₄⁺ (wherein W is C₁₋₄ alkyl) and other amine salts. Physiologically acceptable salts of a hydrogen atom or an amino group include salts or organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids. Physiologically acceptable salts of a compound with a hydroxy group include the anion of said compound in combination with a suitable cation such as Na⁺, NH₄⁺, and NW₄⁺ (wherein W is a C₁₋₄alkyl group). Preferred salts include sodium, calcium, potassium, magnesium, choline, meglumine, hydrochloride, and quaternary ammonium.

The invention includes within its scope all possible stoichiometric and non-stoichiometric forms of the salts of the compounds of Formula (I) or (Ia).

The salts of a compound of Formula (I) or (Ia) may be prepared by contacting appropriate stoichiometric amounts of the free acid with the appropriate base in a suitable solvent. The free acid of a compound of Formula (I) or (Ia) may for example be in solution with the appropriate base added as a solid or both the free acid of a compound of Formula (I) or (Ia) and the appropriate acid may independently be in solution.

Suitable solvents for solubilising a compound of Formula (I) or (Ia) free acid include for example alcohols such as isopropanol; ketones such as acetone; acetonitrile or toluene. If the base is to be added as a solution in a solvent, the solvent used may include acetone, methanol or water.

The salts of a compound of Formula (I) or (Ia) may be isolated in solid form by conventional means from a solution thereof obtained as above. For example, a non-crystalline salt may be prepared by precipitation from solution, spray drying or freeze drying of solutions, evaporating a solution to a glass, or vacuum drying of oils, or solidification of melts obtained from reaction of the free base and the acid.

The salts of a compound of Formula (I) or (Ia) may be prepared by directly crystallising from a solvent in which the salt has limited solubility, or by triturating or otherwise crystallising a non-crystalline salt. For example, organic solvents such as acetone, acetonitrile, butanone, 1-butanol, ethanol, 1-propanol or tetrahydrofuran or mixtures of such solvents may be used. An improved yield of the salts may be obtained by the evaporation of some or all of the solvent or by crystallisation at elevated temperature followed by controlled cooling, for example in stages. Careful control of the precipitation temperature and seeding may be used to improve the reproducibility of the production process and the particle size distribution and form of the product.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of Formula (I) or (Ia) are within the scope of the invention. Therefore, the present invention also relates to solvates of the compounds of Formula (I) or (Ia), for example hydrates.

Salts and solvates of compounds of Formula (I) or (Ia) which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of Formula (I) or (Ia) or salts, solvates or esters thereof and their pharmaceutically acceptable salts and solvates.

Furthermore, some of the crystalline forms of the compounds of Formula (I) or (Ia) or salts and solvates thereof may exist in one or more polymorphic form, which are included in the present invention.

It will be appreciated by those skilled in the art that certain protected derivatives of compounds of Formula (I) or (Ia), which may be made prior to a final deprotection stage, may not possess pharmacological activity as such, but may, in certain instances, be administered orally or parenterally and thereafter metabolised in the body to form compounds defined in the first aspect which are pharmacologically active. Such derivatives may therefore be described as “prodrugs”. All protected derivatives and prodrugs of compounds defined in the first aspect are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compounds of Formula (I) or (Ia). Suitable prodrugs for compounds of Formula (I) or

(la) or salts, solvates or esters thereof include: esters, carbonate esters, hemi-esters, phosphate esters, nitro esters, sulfate esters, sulfoxides, amides, carbamates, azo-compounds, phosphamides, glycosides, ethers, acetals and ketals.

The present invention features a pharmaceutically acceptable ester of a compound of Formula (I) or (Ia).

Esters of the compounds of the present invention are independently selected from the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted by, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

In such esters, unless otherwise specified, any alkyl moiety present advantageously contains from 1 to 18 carbon atoms, particularly from 1 to 6 carbon atoms, more particularly from 1 to 4 carbon atoms, Any cycloalkyl moiety present in such esters advantageously contains from 3 to 6 carbon atoms. Any aryl moiety present in such esters advantageously comprises a phenyl group.

While it is possible for the active ingredient to be administered alone, it is preferable to present it as a pharmaceutical composition. The compositions of the present invention comprise at least one active ingredient, as defined above, together with one or more acceptable carriers thereof and optionally other therapeutic agents. Each carrier must be acceptable in the sense of being compatible with the other ingredients of the composition and not injurious to the patient.

Pharmaceutical compositions include those suitable for oral, rectal, nasal, topical (including transdermal, buccal and sublingual), vaginal or parenteral (including subcutaneous, intramuscular, intravenous, intradermal, and intravitreal) administration. The compositions may conveniently be presented in unit dosage form and may be prepared by any methods well known in the art of pharmacy. Such methods represent a further feature of the present invention and include the step of bringing into association the active ingredients with the carrier, which constitutes one or more accessory ingredients. In general, the compositions are prepared by uniformly and intimately bringing into association the active ingredients with liquid carriers or finely divided solid carriers or both, and then if necessary shaping the product.

The present invention further includes a pharmaceutical composition as hereinbefore defined wherein a compound of the present invention or a pharmaceutically acceptable salt thereof and another therapeutic agent are presented separately from one another as a kit of parts.

Compositions suitable for transdermal administration may be presented as discrete patches adapted to remain in intimate contact with the epidermis of the recipient for a prolonged period of time. Such patches suitably contain the active compound 1) in an optionally buffered, aqueous solution or 2) dissolved and/or dispersed in an adhesive or 3) dispersed in a polymer. A suitable concentration of the active compound is about 1% to 25%, preferably about 3% to 15%. As one particular possibility, the active compound may be delivered from the patch by electrotransport or iontophoresis as generally described in Pharmaceutical Research 3(6), 318 (1986).

Pharmaceutical compositions of the present invention suitable for oral administration may be presented as discrete units such as capsules, caplets, cachets or tablets each containing a predetermined amount of the active ingredients; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredients in a free-flowing form such as a powder or granules, optionally mixed with a binder (e.g. povidone, gelatin, hydroxypropylmethyl cellulose), lubricant, inert diluent, preservative, disintegrant (e.g. sodium starch glycollate, cross-linked povidone, cross-linked sodium carboxymethyl cellulose) surface-active or dispersing agent. Molded tablets may be made by molding a mixture of the powdered compound moistened with an inert liquid diluent in a suitable machine. The tablets may optionally be coated or scored and may be formulated so as to provide slow or controlled release of the active ingredients therein using, for example, hydroxypropylmethyl cellulose in varying proportions to provide the desired release profile. Tablets may optionally be provided with an enteric coating, to provide release in parts of the gut other than the stomach.

Pharmaceutical compositions suitable for topical administration in the mouth include lozenges comprising the active ingredients in a flavored base, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions suitable for vaginal administration may be presented as pessaries, tampons, creams, gels, pastes, foams or spray. Pharmaceutical compositions may contain in addition to the active ingredient such carriers as are known in the art to be appropriate.

Pharmaceutical compositions for rectal administration may be presented as a suppository with a suitable carrier comprising, for example, cocoa butter or a salicylate or other materials commonly used in the art. The suppositories may be conveniently formed by admixture of the active combination with the softened or melted carrier(s) followed by chilling and shaping in molds.

Pharmaceutical compositions suitable for parenteral administration include aqueous and nonaqueous isotonic sterile injection solutions which may contain anti-oxidants, buffers, bacteriostats and solutes which render the pharmaceutical composition isotonic with the blood of the intended recipient; and aqueous and non-aqueous sterile suspensions which may include suspending agents and thickening agents; and liposomes or other microparticulate systems which are designed to target the compound to blood components or one or more organs. The pharmaceutical compositions may be presented in unit-dose or multi-dose sealed containers, for example, ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules and tablets of the kind previously described.

Unit dosage pharmaceutical compositions include those containing a daily dose or daily subdose of the active ingredients, as hereinbefore recited, or an appropriate fraction thereof.

It should be understood that in addition to the ingredients particularly mentioned above the pharmaceutical compositions of this invention may include other agents conventional in the art having regard to the type of pharmaceutical composition in question, for example, those suitable for oral administration may include such further agents as sweeteners, thickeners and flavoring agents.

The present invention features a compound of Formula (I) or (Ia) or pharmaceutically acceptable salt thereof for use in human or veterinary medical therapy, particularly in the treatment of viral infection, particularly flavivirus infection, for example HCV infection.

It will further be appreciated that references herein to treatment or prophylaxis of HCV infection include treatment or prophylaxis of HCV-associated disease such as liver fibrosis, cirrhosis and hepatocellular carcinoma.

The present invention also features a method for the treatment of an animal subject, for example a human subject, with viral infection, particularly HCV infection, which method comprises administering to said subject an effective amount of a compound of Formula (I) or (Ia) or a pharmaceutically acceptable salt thereof.

The present invention also features the use of a compound of Formula (I) or (Ia) or a pharmaceutically acceptable salt thereof in the manufacture of a medicament for the treatment and/or prophylaxis of viral infection, particularly HCV infection.

In general a suitable dose for each of the above-mentioned conditions will be in the range of 0.01 to 250 mg per kilogram body weight of the recipient (e.g. a human) per day, preferably in the range of 0.1 to 100 mg per kilogram body weight per day and most preferably in the range 0.5 to 30 mg per kilogram body weight per day and particularly in the range 1.0 to 20 mg per kilogram body weight per day. Unless otherwise indicated, all weights of active ingredient are calculated as the parent compound of formula (I) or (Ia); for salts or esters thereof, the weights would be increased proportionally. The desired dose may be presented as one, two, three, four, five, six or more sub-doses administered at appropriate intervals throughout the day. In some cases the desired dose may be given on alternative days. These sub-doses may be administered in unit dosage forms, for example, containing 10 to 1000 mg or 50 to 500 mg, preferably 20 to 500 mg, and most preferably 50 to 400 mg of active ingredient per unit dosage form.

Compounds of the present invention are useful as inhibitors of HCV NS3-4A protease. One aspect of the instant invention relates to methods of treating or preventing viral infection, for example an HCV infection, in a biological sample comprising contacting the biological sample with a compound of formula (I) or (Ia) or a pharmaceutically acceptable salt thereof.

The above compounds according to the invention or their pharmaceutically acceptable slats may be employed in combination with other therapeutic agents for the treatment of the above infections or conditions. Combination therapies according to the present invention comprise the administration of a compound of the present invention or a pharmaceutically acceptable salt thereof and another pharmaceutically active agent, for example, interferon, pegylated interferon, and/or ribavirin. The active ingredient(s) and pharmaceutically active agents may be administered simultaneously (i.e., concurrently) in either the same or different pharmaceutical compositions or sequentially in any order. The amounts of the active ingredient(s) and pharmaceutically active agent(s) and the relative timings of administration will be selected in order to achieve the desired combined therapeutic effect.

When a compound of Formula (I) or (Ia) or a pharmaceutically acceptable salt thereof is used in combination with a second therapeutic agent active against the same disease state, the dose of each compound may differ from that when the compound is used alone. Appropriate doses will be readily appreciated by those skilled in the art. It will be appreciated that the amount of a compound of Formula (I) or (Ia) or a salt thereof required for use in treatment will vary with the nature of the condition being treated and the age and the condition of the patient and will be ultimately at the discretion of the attendant physician or veterinarian. The pharmaceutical compositions according to the invention may also be used in combination with other therapeutic agents, for example immune therapies [e.g. interferon, such as interferon alfa-2a (ROFERON®-A; Hoffmann-La Roche), interferon alpha-2b (INTRON®-A; Schering-Plough), interferon alfacon-1 (1NFERGEN®; Intermune), peginterferon alpha-2b (PEGINTRON™; Schering-Plough) or peginterferon alpha-2a (PEGASYS®; Hoffmann-La Roche)], therapeutic vaccines, antifibrotic agents, anti-inflammatory agents [such as corticosteroids or NSAIDs], bronchodilators [such as beta-2 adrenergic agonists and xanthines (e.g. theophylline)], mucolytic agents, anti-muscarinics, anti-leukotrienes, inhibitors of cell adhesion [e.g. ICAM antagonists], anti-oxidants [e.g. N-acetylcysteine], cytokine agonists, cytokine antagonists, lung surfactants and/or antimicrobial, anti-viral agents [e.g. nitazoxanide, ribavirin and amantidine], and anti-HCV agents [for example HCV NS3 protease inhibitors, e.g. TMC435350 (Medivir; Tibotec), BI201335 (Boehringer-Ingelheim), MK-7009 (Merck), VX950 (telapravir; Vertex), SCH503034 (Schering Plough) or ITMN191 (Intermune)], or HCV NS5b polymerase inhibitors [for example, VCH-759 (Virochem) or R7128 (Pharmasset/Roche)], HCV NS5A antagonists [e.g. BMS-790052], RNAi agents or cyclophilin inhibitors (for example DEBIO-025). The compositions according to the invention may also be used in combination with gene replacement therapy.

The combinations referred to above may conveniently be presented for use in the form of a pharmaceutical formulation and thus pharmaceutical formulations comprising a combination as defined herein together with a pharmaceutically acceptable carrier thereof represent a further aspect of the invention.

The individual components of such combinations may be administered either sequentially or simultaneously in separate or combined pharmaceutical formulations. Appropriate doses of known therapeutic agents will be readily appreciated by those skilled in the art.

The present invention further includes the use of a compound according to the invention in the manufacture of a medicament for simultaneous or sequential administration with at least another therapeutic agent, such as those defined hereinbefore.

Compounds of the present invention may be administered with an agent known to inhibit or reduce the metabolism of compounds, for example ritonavir. Accordingly, the present invention features a method for the treatment or prophylaxis of a disease as hereinbefore described by administration of a compound of the present invention in combination with a metabolic inhibitor. Such combination may be administered simultaneously or sequentially. In general a suitable dose for each of the above-mentioned conditions will be in the range of 0.01 to 250 mg per kilogram body weight of the recipient (e.g. a human) per day, preferably in the range of 0.1 to 100 mg per kilogram body weight per day and most preferably in the range 0.5 to 30 mg per kilogram body weight per day and particularly in the range 1.0 to 20 mg per kilogram body weight per day. Unless otherwise indicated, all weights of active ingredient are calculated as the parent compound of formula (I) or (Ia); for salts or esters thereof, the weights would be increased proportionally. The desired dose may be presented as one, two, three, four, five, six or more sub-doses administered at appropriate intervals throughout the day. In some cases the desired dose may be given on alternative days. These sub-doses may be administered in unit dosage forms, for example, containing 1 to 1000 mg or 5 to 500 mg, preferably 2 to 500 mg, and most preferably 1 to 400 mg of active ingredient per unit dosage form.

The compounds of the present invention may be prepared by methods known to one skilled in the art or according to the following reactions schemes and examples, or modifications thereof using readily available starting materials, reagents and conventional synthesis procedures. In these reactions, it is also possible to make use of variants which are known to those of ordinary skill in the art.

### EXAMPLES

### Abbreviations

CDI NAP-carbonyldiimidazole
DBU 1,8-diazabicycloundec-7-ene
DCE 1,1,-dichloroethane
DIAD diisopropylazodicarboxylate
DIEA N,N-di-isopropylethylamine
DMF N,N-dimethylformamide
h hours
HATU 2-(1H-7-azabenzotriazol-1-yl)—1,1,3,3-tetramethyl uronium hexafluorophosphate
THF tetrahydrofuran

### Assays

The potential for compounds of Formula (I) or (Ia) or salts thereof to inhibit NS3-4A HCV protease activity was demonstrated, for example, using the following in vitro assay:

### Hepatitis C NS3 Protease FRET Assay (RET-S1) Using the HCV Genotype 1a NS3-4A Protease Domain

Compounds were tested as inhibitors of HCV NS3-4A protease domain in 10 μl reactions which contained 50 mM HEPES pH 7.5, 20% sucrose, 0.05% NP40, 5 mM DTT, 1 μM 4A peptide (KKGSWIVGRIVLSGKPAIIPKK that served as a cofactor and activated the enzyme in the assay) (prepared by standard synthetic methods), 5 μM RET S1 substrate (Ac-DED(EDANS)EEAbu ψ[COO] ASK(DABCYL)) and 1 nM NS3 protease domain. The RET S1 substrate (Ac-Asp-Glu-Asp(EDANS)-Glu-Glu-Abu-ψ-[COO]-Ala-Ser-Lys(DABCYL)-NH₂) was purchased from Anaspec, Inc. (Taliani et. al, 1996, Anal Biochem). “ψ-[COO]” refers to the location of the ester bond within the molecule

Cleavage of the substrate, which is based on the natural NS4A/NS4B junction (DEMEECASHL), was monitored by measuring the increase in fluorescence with 355 nm excitation/495 nm emission in a Molecular Devices SpectraMax Gemini plate reader. Compounds were dissolved in DMSO and tested at 10 μM in duplicate or in dose response curves with usually 10 μM top concentration and 3-fold dilutions. The maximum level of DMSO was 5%. The standard compound in the assay was the compound BILN-2061 (Lamarre at al., (2003) Nature, 426, 186-189).

Table 1 shows the sequence of the HCV 1a NS3-4A protease domain (SEQ ID NO: 1) compared to the full length HCV 1a 1\153-4A protease (SEQ ID NO: 2). Shaded areas indicate where residues of the NS3-4A protease domain exactly match those of the full length NS3-4A protease.

TABLE 1

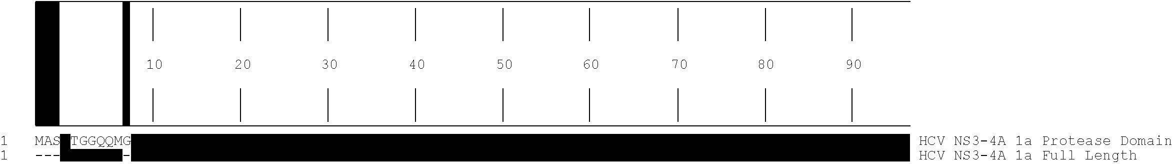

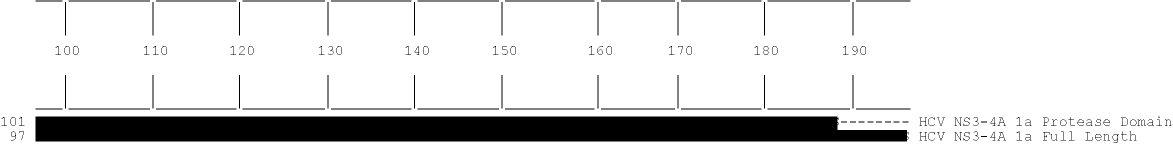

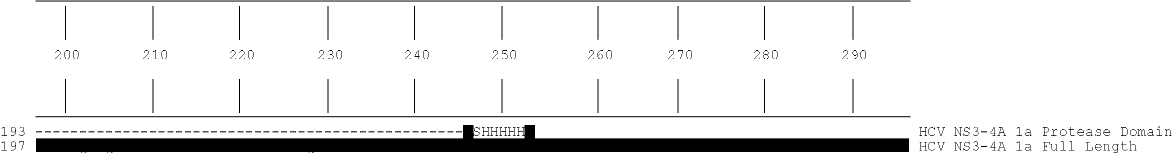

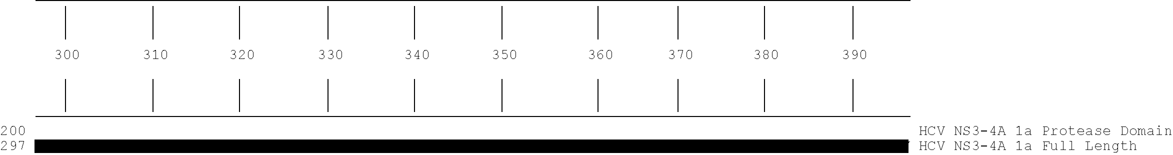

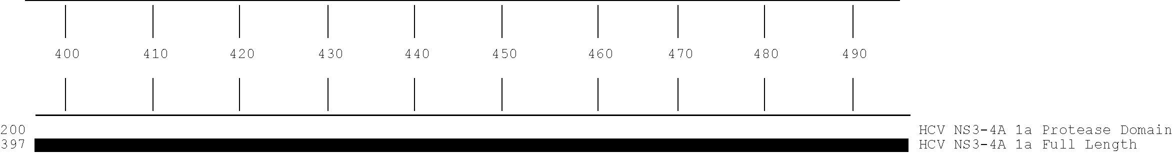

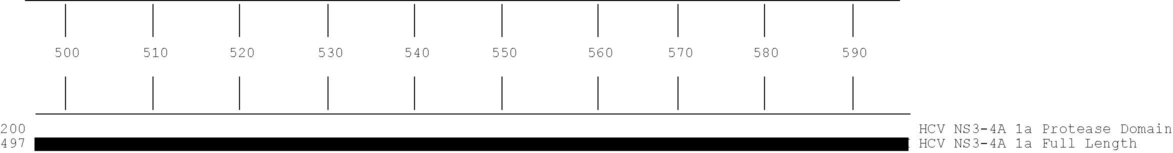

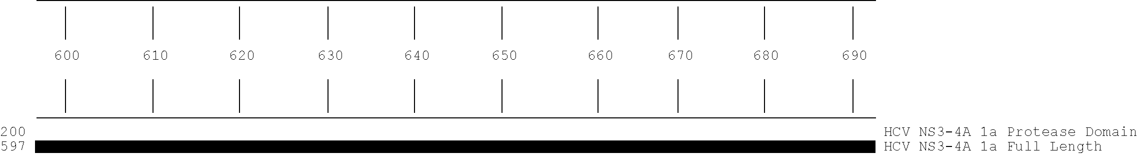

Decoration ‘Decoration #1’: Shade (with deep red at 40% fill) residues that match HCV HS3-4A 1a Full Length exactly.

### Hepatitis C NS3-4A Protease FRET Assay Using the Full Length HCV Genotype 1a NS3-4A Protease

Compounds were tested by the same method as for NS3-4A protease domain, but the full length HCV genotype 1a NS3-4A protease replaced the NS3-4A protease domain.

Compounds were considered active as inhibitors of the protease if they had an IC₅₀ value of less than 10 μM in the above FRET assays.

The compounds of the invention were tested in the FRET Assay using the HCV Genotype la and were found to have an IC₅₀ value of less than 0.1 μM

### Hepatitis C NS3 Protease FRET Assay (QXL520) Using the HCV Genotype 1a NS3-4A Protease Domain

Compounds were tested as inhibitors of HCV NS3-4A protease domain in 10 μl reactions which contained 50 mM HEPES pH 7.5, 20% sucrose, 0.05% NP40, 5 mM DTT, 1 μM 4A peptide (KKGSWIVGRIVLSGKPAIIPKK that served as a cofactor and activated the enzyme in the assay) (prepared by standard synthetic methods), 0.5 μM 5-FAM/QXL520 substrate (Ac-DED(QXL520)EEAbu ψ[COO] ASC(5-FAM)) and 1 nM NS3 protease domain. The 5-FAM/QXL520 substrate (Ac-Asp-Glu-Dap(QXL520)-Glu-Glu-Abu-ψ-[COO]-Ala-Ser-Cys(5-FAM)-NH2) was purchased from Anaspec, Inc. “ψ-[COO]” refers to the location of the ester bond within the molecule

Cleavage of the substrate, which is based on the natural NS4A/NS4B junction (DEMEECASHL), was monitored by measuring the increase in fluorescence with 480 nm excitation/540 nm emission in a PerkinElmer Viewlux imager. Compounds were dissolved in DMSO and tested at 10 μM in duplicate or in dose response curves with usually 10 μM top concentration and 3-fold dilutions. The maximum level of DMSO was 5%. The standard compound in the assay was the compound BILN-2061 (Lamarre et al., (2003) Nature, 426, 186-189).

Compounds of the invention were tested in the FRET Assay using the HCV Genotype 1a

Hepatitis C NS3 Protease FRET Assay (RET-S1) using the HCV Genotype 1a NS3-4A Protease Domain Compounds of Examples 1-5, 10-26, and 29 were found to have an IC₅₀ in the range 1.0-10 nM, for example, the compound of Example 16 had an IC₅₀ of 5 nM.

Hepatitis C NS3 Protease FRET Assay (QXL520) using the HCV Genotype 1a NS3-4A

Protease Domain Compounds of Examples 6-8, 27-28, and 30-36 were found to have an IC₅₀ in the range 0.1-2.0 nM.

### Replicon Luciferase Cell Based Assay

### Method

A 10 mM stock solution in DMSO of each test compound was further diluted in DMSO in the first row of a 384-well, V-bottom microplate, to give 100 times the top concentration of the required dilution series. Dilutions of compound were prepared in 1:3 serial dilutions from the first row onwards robotically. A robot was also used to transfer 0.5 μL volumes from each dilution well into wells of white 384-well assay plates (Nunc #164610). Control wells received 0.5 μL of DMSO alone. Plates were made in duplicate for measuring HCV replication and cytotoxicity in the replicon cell lines.

Suspensions were prepared from cultures of Huh-7 cells stably transfected with sub-genomic HCV NS3—NS5B replicons of either genotype 1b (the ET subline described by Pietschmann, T., Lohmann, V., Kaul, A., Krieger, N., Rinck, G., Rutter, G., Strand, D. & Bartenschlager, R., Journal of Virology, 2002, 76, 4008-4021) or genotype 1a linked to a firefly luciferase reporter gene. Monolayers nearing confluency were stripped from growth flasks with versene-trypsin solution and the cells re-suspended in assay medium comprising DMEM (Invitrogen #11965-092) supplemented with 5% v/v foetal calf serum, 1% v/v non-essential amino acids solution, 100 units/ml penicillin, 100 μg/ml streptomycin and 2 mM GlutaMAX-1. 50 μL of suspension containing 5,000 cells of either genotype 1b or genotype 1a luciferase replicon were added to all wells, except medium controls, of the assay plate with a Multidrop Combi (Thermo Scientific Corporation) and the plate incubated for 48 hours at 37° C. in a 5% CO₂ atmosphere. A solution of Steady-Glo cytolytic buffer/luciferase substrate (Promega #E2550) was prepared according to the manufacturer's instructions, and 20 μL were added to each well with a Multidrop Combi. To measure cytotoxicity, a solution of CellTiter-Glo Luminescent Cell Viability Assay reagent (Promega #G7573) was prepared according to the manufacturer's instructions, and 20 ul were added to each well with a Multidrop Combi. The plates were then read for luminescence on an Envision Multilabel Reader (Perkin Elmer)

### HCV PI Transient Replicon Assay Protocol:

A model system for HCV RNA replication is a cell based assay using subgenomic or genomic HCV replicons containing HCV genes and possibly a selectable and/or screenable marker. The HCV replicon is a self-replicating RNA that does not produce infectious particles. The HCV PI transient replicons assay was derived from two publications (Science 258, 110, 1999; J Virol 75, 12047, 2001). In this assay, the target cells were “ET Cured cells”, a cell line generated by treating ET replicons cells (RebliKon) with Interferon a for several passages until the HCV genome was cleared. Other modifications to the published protocol included 1) cells were transfected in PBS and 2) were treated for 72 hours with compounds. Data from dose responses was analyzed using BioAssay. EC50 values were generated by plotting percent inhibition against compound concentration. The various HCV mutant replicons were generated by standard molecular biology techniques and confirmed by sequencing.

### Data Analysis

Toxicity: The luminescence values from all compound-free wells containing cells were averaged to obtain a positive control value. The mean luminescent value from the compound-free wells that had received no cells was used to provide the negative (background) control value. After the subtraction of the mean background from all values, the readings from wells at each compound concentration were expressed as a percentage of the positive control signal. The BioAssay application (CambridgeSoft) and XC50 module were used to plot the curve of percentage inhibition against compound concentration and derive the 50% toxic concentration (CClC₅₀) for the compound.

Potency: The luminescence values from all compound-free wells containing cells were averaged to obtain a positive control value. The mean luminescence value from the compound-free wells that had received no cells was used to provide the negative (background) control value. After the subtraction of the mean background from all values, the readings from wells at each compound concentration were expressed as a percentage of the positive control signal. The quantifiable and specific reduction of luciferase signal in the presence of a drug is a direct measure of replicon inhibition. The BioAssay application and XC50 module were used to plot the curve of percentage inhibition against compound concentration and derive the 50% inhibitory concentration (IC₅₀) for the compound.

The following compounds of the Invention were tested in the above replicon assays:

TABLE 2

| | Genotype | Genotype |
|---|---|---|
| Example No. | 1a | 1b |
| Example 1 | *** | *** |
| Example 2 | *** | *** |
| Example 3 | *** | *** |
| Example 4 | *** | *** |
| Example 5 | *** | *** |
| Example 6 | *** | *** |
| Example 7 | *** | *** |
| Example 8 | *** | *** |
| Example 9 | *** | *** |
| Example 10 | *** | *** |
| Example 11 | *** | *** |
| Example 12 | *** | *** |
| Example 13 | *** | *** |
| Example 14 | *** | *** |
| Example 15 | *** | *** |
| Example 16 | *** | *** |
| Example 17 | *** | *** |
| Example 18 | *** | *** |
| Example 19 | *** | *** |
| Example 20 | *** | *** |
| Example 21 | *** | *** |
| Example 22 | *** | *** |
| Example 23 | *** | *** |
| Example 24 | *** | *** |
| Example 25 | *** | *** |
| Example 26 | *** | *** |
| Example 27 | *** | *** |
| Example 28 | *** | *** |
| Example 29 | *** | *** |
| Example 30 | *** | *** |
| Example 31 | *** | *** |
| Example 32 | *** | *** |
| Example 33 | *** | *** |
| Example 34 | *** | *** |
| Example 35 | *** | *** |
| Example 36 | *** | *** |

Key (IC₅₀ μM):
* 1 μM-0.1 μM
** 0.1 μM-0.01 μM
*** <0.01 μM

Compounds of the invention are thought to be active against HCV protease-resistant mutants. In addition, example 16 and selected compounds in this application were demonstrated to have an improved profile (e.g. A1566S, A156T, R155K, others) compared with other HCV PI inhibitors ITMN-191 and TMC-435350 with respect to). EC₅₀ values (nM) are shown below in Table 3.

TABLE 3

| | wt | A156S | A156T | A156V | D168A | D168V | R155K |
|---|---|---|---|---|---|---|---|
| | EC₅₀ | EC₅₀ | EC₅₀ | EC₅₀ | EC₅₀ | EC₅₀ | EC₅₀ |
| | values (nM) | values (nM) | values (nM) | values (nM) | values (nM) | values (nM) | values (nM) |
| ITMN-191 | 0.07 | 1.64 | 6.46 | 11.9 | 54.4 | 33.1 | 109 |
| TMC-435350 | 0.87 | 0.28 | 57.54 | 36.3 | 161 | 43.7 | 58.9 |
| Example 16 | 0.04 | 0.07 | 1.95 | 7.17 | 24.8 | 80.6 | 6.93 |

Compounds of the present invention may be made by the following scheme or by methods known to those skilled in the art.

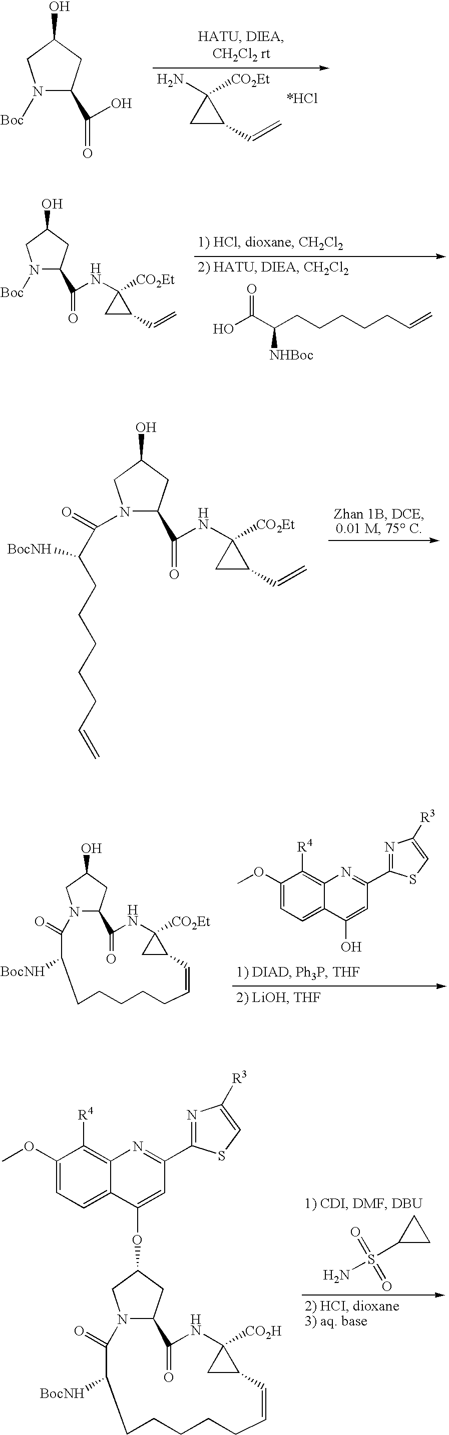

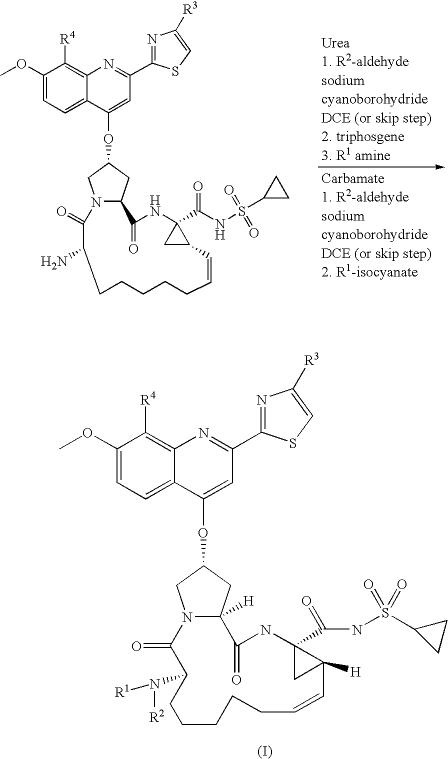

### Example 1

### (2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-{[(Phenylamino)carbonyl]amino}-1,2,3,6,7,8,9,10,11,13a,14,15,16,16a-tetradecahydrocyclopropa[e]pyrrolo[1,2-a][1,4]diazacyclopentadecine-14a(5H)-carboxamide

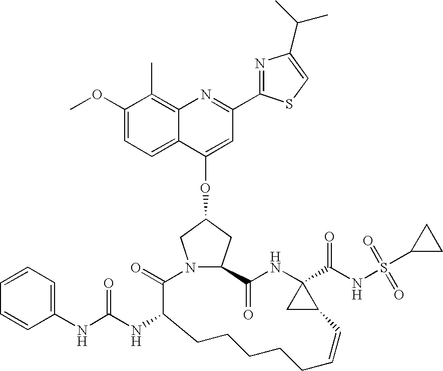

### Preparation of (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11 ,13a,14,15,16,16a-tetraclecahydrocyclopropa[e]pyrrolo[1,2-][1,4]diazacyclopentadecine-14a(5H)-carboxamide dihydrochloride 5

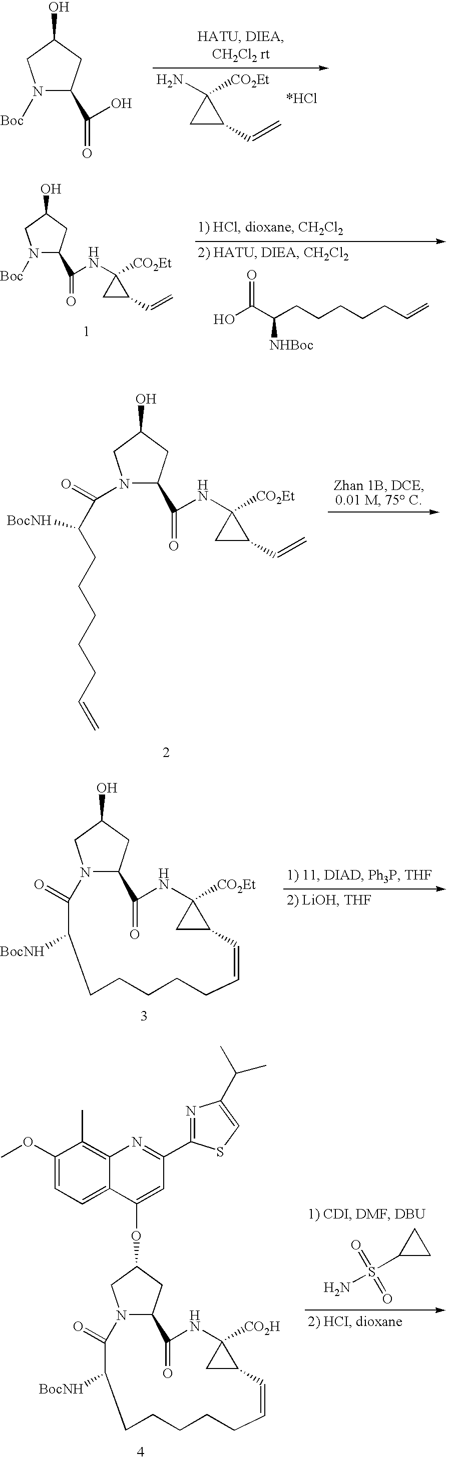

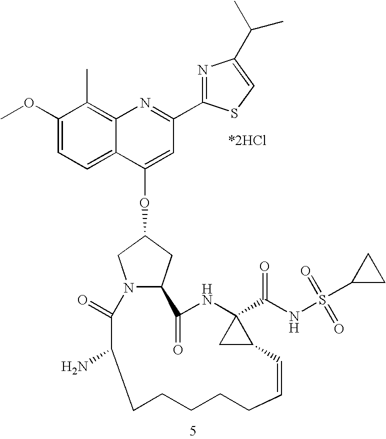

### Intermediate 1

### 1,1-dimethylethyl(2S,4S)-2-[({(1R,2S)-2-ethenyl-1-[(ethyloxy)carbonyl]cyclopropyl}amino)carbonyl]-4-hydroxy-1-pyrrolidinecarboxylate

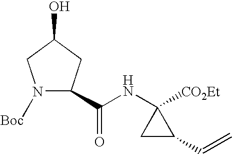

To a solution of (4S)-1-{[(1,1-dimethylethyl)oxy]carbonyl}-4-hydroxy-L-proline (1.80 g, 7.8 mmol), ethyl (1R,2S)-1-amino-2-ethenylcyclopropanecarboxylate hydrochloride (1.49 g, 7.8 mmol) and HATU (3.26 g, 8.6 mmol) in dichloromethane (80 mL) which had been cooled to 0° C. was added diisopropylethylamine (2.9 mL, 16.4 mmol) and the solution was stirred at 0° C. for 30 min and them room temperature for 5 h under a nitrogen atmosphere. The reaction was concentrated in vacuo and purified by silica gel chromatography eluting with 70-100% hexanes/ethyl acetate. The eluent was concentrated to about 200 mL and washed with 10% aqueous potassium carbonate solution (50 mL) and the organic layer dried (MgSO₄) and concentrated in vacuo to afford intermediate 1 as a white foam (2.72 g, 95% yield).

1H NMR (400 MHz, DMSO-d₆) δ ppm 1.08-1.19 (m, 3H) 1.24 (dd, J=9.33, 5.12 Hz, 1H) 1.28-1.45 (m, 9H) 1.54-1.67 (m, 1H) 1.67-1.81 (m, 1H) 2.05-2.20 (m, 1H) 2.20-2.41 (m, 1H) 3.05-3.22 (m, 1H) 3.41-3.57 (m, 1H) 3.91-4.09 (m, 3H) 4.07-4.23 (m, 1H) 4.98-5.19 (m, 2H) 5.19-5.32 (m, 1H) 5.40-5.82 (m, 1H) 8.55-8.87 (m, 1H). LC-MS (APCI): m/z 369.19 (M+H)⁺.

### Intermediate 2

### ethyl(1R,2S)-1-({(4S)-1-[(2S)-2-({[(1,1-dimethylethyl)oxy]carbonyl}amino)-8-nonenoyl]-4-hydroxy-L-prolyl}amino)-2-ethenylcyclopropanecarboxylate

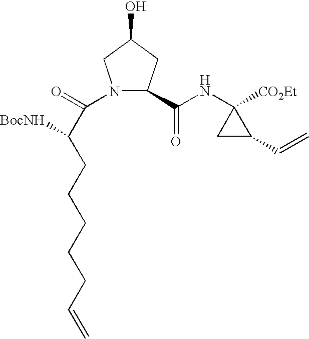

To a solution of 1,1-dimethylethyl(2S,4S)-2-[({(1R,2S)-2-ethenyl-1-[(ethyloxy)carbonyl]cyclopropyl}amino)carbonyl]-4-hydroxy-1-pyrrolidinecarboxylate

Intermediate 1 (2.70 g, 7.3 mmol) in dichloromethane (75 mL) was added 4N HCl in dioxane (18 mL) and the solution was stirred at room temperature for 1.5 h under a nitrogen atmosphere. The solution was concentrated in vacuo and the residue suspended in ethyl acetate and concentrated in vacuo. The residue was suspended in dichloromethane (50 mL) and HATU (3.05 g, 8.0 mmol) was added followed by a solution of (2R)-2-({[(1,1-dimethylethyl)oxy]carbonyl}amino)-8-nonenoic acid (2.0 g, 7.3 mmol) in dichloromethane (25 mL). The reaction was cooled to 0° C. and diisopropylethylamine (4.1 mL, 23.4 mmol) was added dropwise. The reaction was stirred under an atmosphere of nitrogen at 0° C. for 30 min and room temperature for 3.5 h. The reaction was concentrated in vacuo and purified by silica gel chromatography eluting with 50-100% hexanes/ethyl acetate to afford intermediate 2 as an oil (3.37 g, 88% yield). 1H NMR (400 MHz, DMSO-d₆) δ ppm 1.06-1.18 (m, 3H) 1.18-1.51 (m, 17H) 1.50-1.65 (m, 2H) 1.70 (dt, J=12.84, 6.42 Hz, 1H) 1.89-2.13 (m, 3H) 2.18-2.40 (m, 1H) 3.91-4.11 (m, 4H) 4.10-4.30 (m, 3H) 4.80-5.13 (m, 3H) 5.15-5.27 (m, 1H) 5.31 (d, J=6.02 Hz, 1H) 5.62 (ddd, J=17.11, 9.83, 9.58 Hz, 1H) 5.69-5.89 (m, 1H) 6.65-7.06 (m, 1H) 8.48-8.76 (m, 1H). LC-MS (APCI): m/z 522.36 (M+H)⁺.

### Intermediate 3

### ethyl(2S,6S,13aS,14aR,16aS)-6-({[(1,1-dimethylethyl)oxy]carbonyl}amino)-2-hydroxy-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

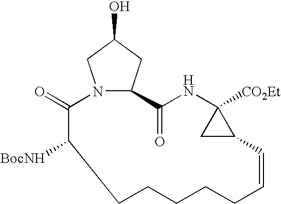

To a solution of ethyl(1R,2S)-1-({(4S)-1-[(2S)-2-({[(1,1-dimethylethyl)oxy]carbonyl}amino)-8-nonenoyl]-4-hydroxy-L-prolyl}amino)-2-ethenylcyclopropanecarboxylate intermediate 2 (3.37 g, 6.5 mmol) in anhydrous 1,2-dichloroethane (700 mL) was added Zhan 1B catalyst (950 mg, 1.3 mmol) and the reaction was evacuated and purged with nitrogen several times. The reaction was heated to 75° C. under a nitrogen atmosphere with mechanical stirring for 21 h. The reaction was concentrated in vacuo and purified by silica gel chromatography eluting with 50-100% hexanes/ethyl acetate to afford intermediate 3 as a brown solid (1.98 g, 62% yield). 1H NMR (400 MHz, DMSO-d₆) δ ppm 1.05-1.22 (m, 3H) 1.22-1.44 (m, 12H) 1.44-1.50 (m, 1H) 1.51-1.59 (m, 1H) 1.58-1.72 (m, 1H) 1.78 (br. s., 2H) 2.00-2.16 (m, 1H) 2.21-2.44 (m, 4H) 2.53-2.61 (m, 1H) 3.34-3.46 (m, 1H) 3.84-4.06 (m, 4H) 4.07-4.17 (m, 1H) 4.23 (br. s., 2H) 5.19-5.32 (m, 1H) 5.43-5.63 (m, 2H) 6.76-7.03 (m, 1H) 8.81 (s, 1H). LC-MS (APCI): m/z 494.34 (M+H)⁺.

### Intermediate 4

### (2R,6S,13aS,14aR,16aS)-6-({[(1,1-dimethylethyl)oxy]carbonyl}amino)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

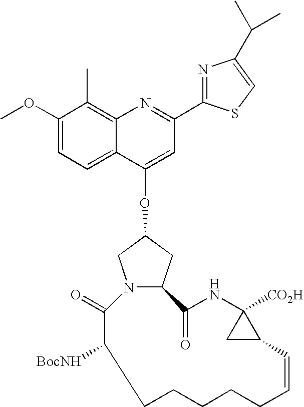

A solution of ethyl(2S,6S,13aS,14aR,16aS)-6-({[(1,1-dimethylethyl)oxy]carbonyl}amino)-2-hydroxy-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate intermediate 3 (1.87 g, 3.8 mmol), 8-methyl-2-[4-(1-methylethyl)-1,3-thiazol-2-yl]-7-(methyloxy)-4-quinolinol 11 (1.43 g, 4.5 mmol), and triphenylphosphine (1.19 g, 4.5 mmol) in anhydrous toluene (60 mL) was concentrated in vacuo to remove trace water. The residue was dissolved in anhydrous THF (20 mL) and cooled to 0° C.

Diisopropylazodicarboxylate (0.9 mL, 4.5 mmol) was added dropwise and the ice bath removed, and the reaction was stirred at room temperature for 18 h under an atmosphere of nitrogen. The reaction was quenched with water (20 mL) and partitioned between an equal volume of ethyl acetate and water. The organic layer was separated and dried (MgSO₄) and concentrated in vacuo. The residue was purified by silica gel chromatography eluting with 20-70% hexanes/ethyl acetate. The residue obtained was dissolved in a mixture of THF (12 mL), water (6 mL) and methanol (6 mL) to which was added lithium hydroxide (530 mg, 23 mmol) and the reaction stirred at room temperature for 18 h. The reaction was treated with 1N HCl (25 mL) and partitioned between an equal volume of ethyl acetate and water. The organic layer was separated and dried (MgSO₄) and concentrated in vacuo to afford intermediate 4 as tan solid (1.95 g, 61% yield). 1H NMR (400 MHz, DMSO-d₆) δ ppm 1.17 (s, 6H) 1.25-1.42 (m, 15H) 1.41-1.55 (m, 2H) 1.73 (br. s., 2H) 2.10-2.26 (m, 2H) 2.30-2.43 (m, 2H) 2.58 (s, 3H) 3.06-3.23 (m, 1H) 3.84-3.92 (m, 2H) 3.93 (s, 3H) 4.42-4.50 (m, 1H) 4.53-4.64 (m, 1H) 5.13-5.33 (m, 1H) 5.45-5.58 (m, 1H) 5.64 (br. s., 2H) 7.05 (br. s., 1H) 7.26-7.36 (m, 1H) 7.46 (s, 1H) 7.51 (s, 1H) 8.05 (d, J=9.43 Hz, 1H) 8.65 (s, 1H) 12.18 (br. s., 1H). LC-MS (ESI): m/z 762.58 (M+H)⁺.

### Intermediate 5

### (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide dihydrochloride

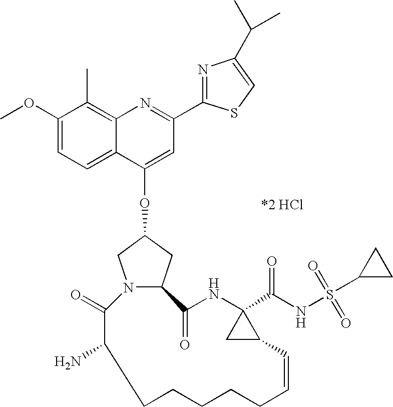

To a solution of (2R,6S,13aS,14aR,16aS)-6-({[(1,1-dimethylethyl)oxy]carbonyl}amino)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid intermediate 4 (1.75 g, 2.3 mmol) in anhydrous DMF (15 mL) was added N,N′-carbonyldiimidazole (410 mg, 2.5 mmol) and the reaction heated to 40° C. for 1 h under an atmosphere of nitrogen.

Cyclopropanesulfonamide (450 mg, 3.7 mmol) and 1,8-diazabicyclo[5.4.0]-undec-7-ene (0.37 mL, 2.5 mmol) were added and the reaction stirred at 40° C. for 18 h under an atmosphere of nitrogen. The reaction was diluted with ethyl acetate (150 mL) and washed with 0.1 N HCl (150 mL). The organic layer was separated and dried (MgSO₄) and concentrated in vacuo. The residue was dissolved in dry dichloromethane (20 mL) and treated with 4N HCl in dioxane (5 mL) and the reaction stirred at room temperature for 3 h under an atmosphere of nitrogen. The reaction was concentrated in vacuo, triturated with ethyl acetate and filtered to afford intermediate 5 (1.2 g, 60% yield) as a yellow solid which was used in subsequent reactions without further purification. 1H NMR (400 MHz, DMSO-d₆) δ ppm 0.96-1.10 (m, 2H) 1.30-1.37 (m, 6H) 1.37-1.52 (m, 4H) 1.51-1.64 (m, 4H) 1.63-1.93 (m, 4H) 2.14-2.28 (m, 1H) 2.59 (s, 3H) 2.65-2.78 (m, 2H) 2.89-2.97 (m, 2H) 3.07-3.22 (m, 1H) 3.97 (s, 3H) 3.99-4.11 (m, 2H) 4.19-4.36 (m, 2H) 4.43-4.57 (m, 1H) 5.11-5.24 (m, 1H) 5.57-5.66 (m, 1H) 5.72 (br. s., 1H) 7.44 (d, J=9.43 Hz, 1H) 7.49 (s, 1H) 7.55 (s, 1H) 8.06 (d, J=9.03 Hz, 1H) 8.23 (br. s., 3H) 9.10 (s, 1H) 11.10 (s, 1H). LC-MS (ESI): m/z 765.35 (M+H)⁺.

### Preparation of 8-methyl-2-[4-(1-methylethyl)-1,3-thiazol-2-yl]-7-(methyloxy)-4-quinolinol 11

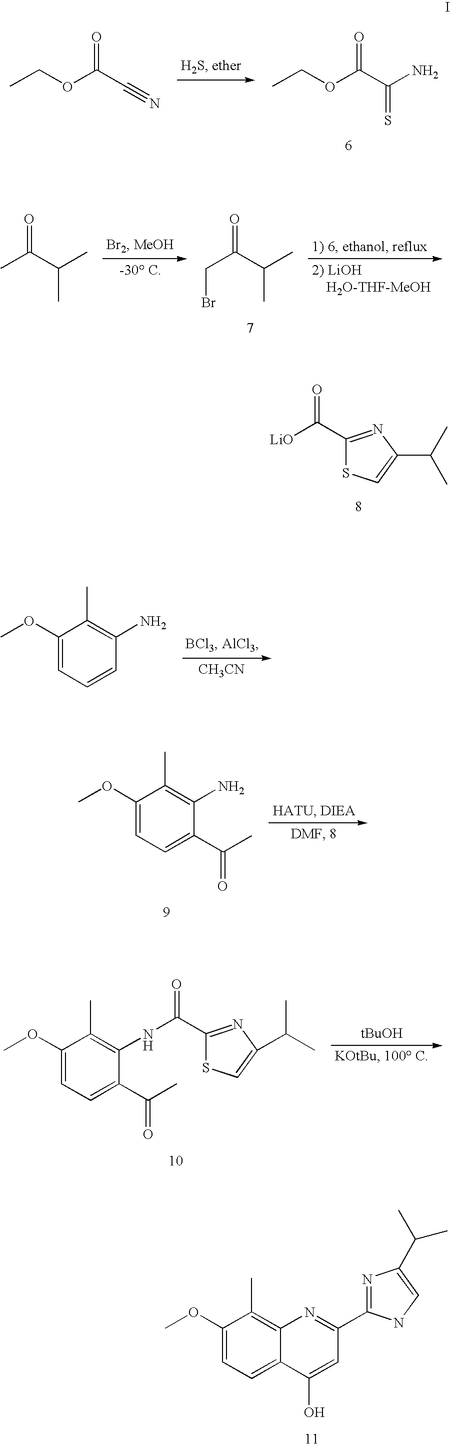

### Intermediate 6

### ethyl amino(thioxo)acetate

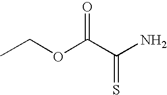

To a solution of [(cyanocarbonyl)oxy]ethane (25 g, 0.25 mol) mixed with triethylamine (1 ml) in ether (200 ml) cooled to 0° C. was bubbled H₂S for 2 hours. Resulting solution was stirred at RT overnight before charging with N₂. 1N HCl (200 ml) was introduced and stirred for another 30 min. Extracted with ether and separated. Organic layer was washed with brine and dried over MgSO₄. Filtered and evaporated to afford ethyl amino(thioxo)acetate 6 (32g , 95%) as a yellow solid. ¹H NMR (400 MHz, CHLOROFORM-d) δ ppm 1.37 (t, J=7.14 Hz, 3H) 4.34 (q, J=7.14 Hz, 2H) 7.49-8.43 (m, 2H), LC-MS (ESI): m/z 134 (M+H)⁺.

### Intermediate 7

### 1-bromo-3-methyl-2-butanone

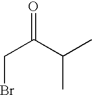

To a solution of methyl isopropyl ketone (10.0 g, 114 mmol) in anhydrous methanol (110 mL) cooled to −30° C. under a nitrogen atmosphere was added bromine (5.8 mL, 114 mmol) dropwise and the reaction was allowed to warm to room temperature and stirred for 3 h. The reaction was concentrated in vacuo diluted with diethyl ether and washed with aqueous saturated NaHCO₃ solution followed by brine, dried (MgSO₄) and concentrated in vacuo. The residue was purified by silica gel chromatography eluting with 0-60% hexanes/dichloromethane to afford intermediate 7 as a clear oil which was used without further purification. (12.46 g, 54% yield). 1H NMR (400 MHz, CHLOROFORM-d) δ ppm 1.07-1.20 (m, 6H) 2.96 (dt, J=13.79, 6.85 Hz, 1H) 3.96 (s, 2H).

### Intermediate 8

### 4-(1-methylethyl)-1,3-thiazole-2-carboxylate, lithium salt

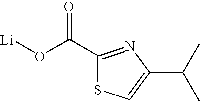

To a solution of ethyl amino(thioxo)acetate 6 (4.16 g, 31.3 mmol) in ethanol (30 mL) was added 1-bromo-3-methyl-2-butanone 7 (5.16 g, 31.3 mmol) and the solution was heated to reflux for 1.5 h under a nitrogen atmosphere. The reaction was cooled to room temperature and solid NaHCO₃ was added until gas evolution ceased. Water (50 mL) was added and the aqueous layer was extracted with ethyl acetate. The organic layer was washed with brine, dried (MgSO₄) and concentrated in vacuo. The reaction was repeated in an identical fashion on a 27.6 mmol scale and combined to afford 7.46 g of an orange oil. The oil was dissolved in THF (70 mL), water (15 mL) and methanol (25 mL) followed by the addition of anhydrous lithium hydroxide (1.3 g, 54.3 mmol) and the solution was stirred at room temperature for 18 h. The reaction was concentrated in vacuo and the residue triturated in diethyl ether and filtered to afford intermediate 8 as a white solid (5.26 g, 50% yield). 1H NMR (400 MHz, METHANOL-d₄) δ ppm 1.28 (d, J=7.02 Hz, 6H) 3.08 (ddd, J=13.34, 6.82, 6.72 Hz, 1H) 7.13 (d, J=0.80 Hz, 1H). LC-MS (ESI): m/z 170.22 (M−Li)⁻.

### Intermediate 9

### 1-[2-amino-3-methyl-4-(methyloxy)phenyl]ethanone

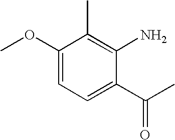

To a solution of 2-methyl-3-(methyloxy)aniline (5.0 g, 36.5 mmol) in p-xylene (80 mL) and cooled to 0° C., added BCl₃ (36.5 mL) as a 1M solution in dichloromethane and stirred for 30 min at 0° C. under a nitrogen atmosphere. Acetonitrile (2.6 mL, 54.7 mmol) was added and the solution was stirred for an additional 30 min at 0° C. AlCl₃ (4.87 g, 36.5 mmol) was suspended in anhydrous dichloromethane (50 mL) under a nitrogen atmosphere and cooled to 0° C. The xylene solution was transferred to an addition funnel and added to the dichloromethane reaction dropwise. The reaction was stirred at 0° C. for 45 min and the dichloromethane was removed in vacuo. The xylene solution was heated to 70° C. for 18 h and cooled to room temperature. Water (80 mL) was added and the solution was heated to 70° C. for 1 h and cooled to room temperature. The reaction was diluted with ethyl acetate and the aqueous layer separated. The organic layer was washed with an equal volume of water, brine and dried (MgSO₄) and concentrated in vacuo. To the residue was added 5:1 hexanes:diethyl ether and the precipitate collected by filtration to afford intermediate 9 as a white solid (4.66 g, 71% yield). 1H NMR (400 MHz, DMSO-d₆) δ ppm 1.87 (s, 3H) 2.42 (s, 3H) 3.77 (s, 3H) 6.30 (d, J=9.04 Hz, 1H) 7.03 (br. s., 2H) 7.65 (d, J=9.04 Hz, 1H). LC-MS (ESI): m/z 180.10 (M+H)⁺.

### Intermediate 10

### N-[6-acetyl-2-methyl-3-(methyloxy)phenyl]-4-(1-methylethyl)-1,3-thiazole-2-carboxamide

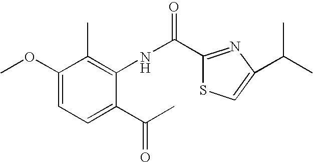

To a solution of 4-(1-methylethyl)-1,3-thiazole-2-carboxylic acid lithium salt 8 (2.86 g, 0.016 mmol) in DMF (42 ml) was added HATU (11.03 g, 0.029 mmol) and stirred for 20 min at RT before adding 1-[2-amino-3-methyl-4-(methyloxy)phenyl]ethanone 9 (2.595 g, 0.0145 mmol). Stirring was continued overnight . Reaction mixture was partitioned in water and ethyl acetate. Separated and the aqueous layer was extracted with ethyl acetate till no product was detected in water phase. Combined the organic phases and dried over MgSO₄. Filtered , concentrated and purified by silica gel chromatography (ethyl acetate/hexane, 0-50%) to afford N-[6-acetyl-2-methyl-3-(methyloxy)phenyl]-4-(1-methylethyl)-1,3-thiazole-2-carboxamide 10 as a solid (1.92 g, 40%). ¹H NMR (400 MHz, CHLOROFORM-d) δ ppm 1.39 (d, J=6.78 Hz, 6H), 2.15 (s, 3H), 2.57 (s, 3H), 3.21 (s, 1H), 3.91 (s, 3H), 6.77 (d, J=8.61 Hz, 1H), 7.15 (s, 1H), 7.74 (d, J=8.61 Hz, 1H), 11.28 (s, 1H), LC-MS (ESI): m/z 333 (M+H)⁺

### Intermediate 11

### 8-methyl-2-[4-(1-methylethyl)-1,3-thiazol-2-yl]-7-(methyloxy)-4-quinolinol

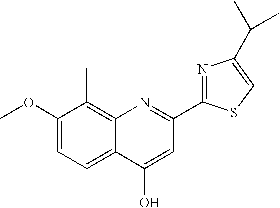

To a solution of N-[6-acetyl-2-methyl-3-(methyloxy)phenyl]-4-(1-methylethyl)-1,3-thiazole-2-carboxamide 10 (3.22 g, 9.7 mmol) in anhydrous tert-butanol (90 mL) was added solid potassium tert-butoxide (2.3 g, 20.4 mmol) and the reaction was heated to reflux under a nitrogen atmosphere for 18 h. The reaction was cooled to room temperature, the tert-butanol removed in vacuo and the residue partitioned between ethyl acetate and an equal volume of 0.15 N aqueous HCl. The aqueous layer was extracted with ethyl acetate and the combined organic layers were washed with brine and dried (MgSO₄) and concentrated in vacuo to afford intermediate 11 (3.03 g, 99% yield) as a brown solid. 1H NMR (400 MHz, CHLOROFORM-d) δ ppm 1.31-1.41 (m, 6H) 2.42 (s, 3H) 3.17 (dt, J=13.49, 6.80 Hz, 1H) 3.97 (s, 3H) 6.83 (br. s., 1H) 7.02 (d, J=9.03 Hz, 1H) 7.09 (s, 1H) 8.24 (d, J=8.83 Hz, 1H) 9.63 (br. s., 1H). LC-MS (ESI): m/z 315.14 (M+H)⁺.

### Example 1

### (2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-{[(phenylamino)carbonyl]amino}-1,2,3,6,7,8,9,10,11,13a,14,15,16,16a-tetradecahydrocyclopropa[e]pyrrolo[1,2-a][1,4]diazacyclopentadecine-14a(5H)-carboxamide

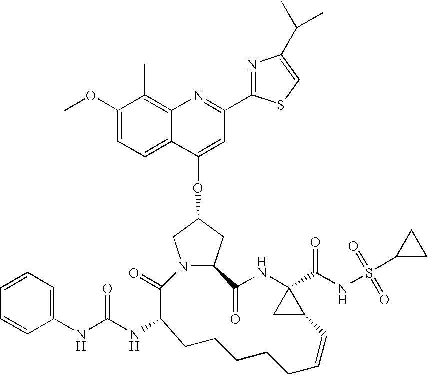

To a suspension of (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide dihydrochloride 5 (50 mg, 0.06 mmol) in anhydrous THF (0.6 mL) was added diisopropylethylamine (0.03 mL, 0.18 mmol) followed by phenylisocyanate (0.007 mL, 0.066 mmol) and the solution stirred at room temperature under a nitrogen atmosphere for 4 h. The reaction was concentrated in vacuo and purified by HPLC eluting with 10-90% acetonitrile/water/0.1% formic acid to afford (2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-{[(phenylamino)carbonyl]amino}-1,2,3,6,7,8,9,10,11,13a,14,15,16,16a-tetradecahydrocyclopropa[e]pyrrolo[1,2-a][1,4]diazacyclopentadecine-14a(5H)-carboxamide as a yellow solid (29 mg, 55% yield).

1H NMR (400 MHz, DMSO-d₆) δ ppm 0.93-1.13 (m, 4H) 1.23 (br. s., 2H) 1.34 (d, J=6.82 Hz, 6H) 1.36-1.51 (m, 4H) 1.51-1.60 (m, 2H) 1.65-1.82 (m, 2H) 2.28-2.46 (m, 2H) 2.57 (s, 3H) 2.60-2.75 (m, 2H) 2.90 (br. s., 1H) 3.09-3.21 (m, 1H) 3.86 (s, 3H) 3.91-4.00 (m, 1H) 4.04 (s, 2H) 4.21-4.43 (m, 2H) 4.66-4.81 (m, 1H) 5.08-5.24 (m, 1H) 5.52-5.62 (m, 1H) 5.65 (br. s., 1H) 6.47 (d, J=7.42 Hz, 1H) 6.84-6.95 (m, 1H) 7.02 (br. s., 1H) 7.17 (t, J=7.83 Hz, 2H) 7.21-7.28 (m, 2H) 7.47 (s, 1H) 7.54 (s, 1H) 8.06 (d, J=9.23 Hz, 1H) 8.45 (br. s., 1H) 8.78 (br. s., 1H) 11.09 (br. s., 1H). HRMS for C₄₅H₅₄N₇O₈S₂ (M+H)⁺ calc: 884.3475, found: 884.3497.

### Example 2

### (2R,6S,13aS,14aR,16aS)-N-(cyclopropylsulfonyl)-6-{[(diethylamino)carbonyl]amino}-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

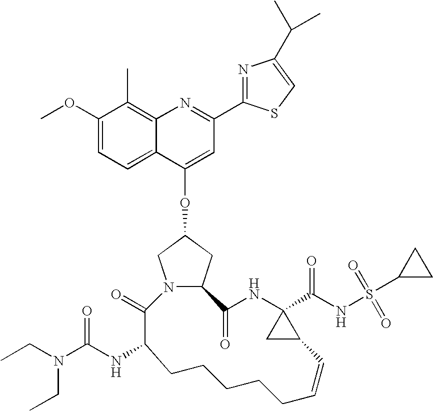

At 0° C., to a stirred solution of (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide dihydrochloride (5) (50 mg, 0.06 mmol) in THF (5 mL) were added N,N-diisopropylethylamine (34 mg, 0.3 mmol) and triphosgene (12 mg, 0.04 mmol). The resulting mixture was stirred while it naturally warmed up to room temperature over 6 hours before addition of diethyl amine (22 mg, 0.3 mmol). Stirring was continued for additional 4 hours. After solvents was evaporated, the crude product was purified by RP HPLC(C-18, 20 to 90% acetonotrile/water (0.1% formic acid)) to give (2R,6S,13aS,14aR,16aS)-N-(cyclopropylsulfonyl)-6-{[(diethylamino)carbonyl]amino}-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide as white foam (36 mg, yield 69%). 1H NMR (400 MHz, METHANOL-d₄) δ ppm 8.08 (d, J=9.03 Hz, 1H) 7.60 (s, 1H) 6.89-7.30 (m, 2H) 5.59-5.75 (m, 1H) 5.55 (br. s., 1H) 5.06 (t, J=9.43 Hz, 1H) 4.55-4.75 (m, 2H) 4.40 (dd, J=10.83, 2.61 Hz, 1H) 4.09 (dd, J=11.54, 3.51 Hz, 1H) 3.92 (s, 3H) 3.03-3.23 (m, 5H) 2.88 (ddd, J=6.32, 3.11, 3.01 Hz, 1H) 2.71-2.81 (m, 1H) 2.57 (s, 5H) 2.38 (d, J=8.63 Hz, 1H) 1.78-2.04 (m, 2H) 1.70 (dd, J=8.03, 5.42 Hz, 1H) 1.39-1.62 (m, 6H) 1.15-1.40 (m, 9H) 1.03-1.15 (m, 2H) 0.97 (t, 7H) LC-MS (ESI): m/z 865.7 (M+H)⁺.

### Example 3

### (2R,6S,13aS,14aR,16aS)-6-[(aminocarbonyl)amino]-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

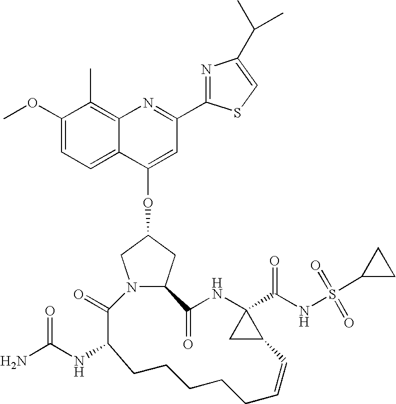

(2R,6S,13aS,14aR,16aS)-6-[(aminocarbonyl)amino]-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (27.8 mg, yield 58%) was obtained as a foam from 2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide dihydrochloride (5) (50 mg, 0.06 mmol), triphosgene (12 mg, 0.04 mmol) and aqueous ammonia by the similar procedure outlined in Example 2. 1H NMR (400 MHz, METHANOL-d₄) δ ppm 8.05 (d, J=6.02 Hz, 3H) 7.58 (s, 1H) 7.19-7.38 (m, 3H) 5.58-5.79 (m, 1H) 5.47-5.58 (m, 1H) 4.96-5.14 (m, 1H) 4.59 (s, 3H) 4.29-4.49 (m, 2H) 4.03-4.26 (m, 2H) 3.93 (s, 4H) 3.06-3.24 (m, 2H) 2.81-3.00 (m, 2H) 2.65-2.81 (m, 2H) 2.29-2.49 (m, 2H) 1.74-1.98 (m, 2H) 1.63-1.74 (m, 1H) 1.36 (m, 15H) 1.07 (br. s., 2H) 0.96 (none, 1H)LC-MS (ESI): m/z 808.6 (M+H)⁺.

### Example 4

### (2R,6S,13aS,14aR,16aS,Z)-2-(8-chloro-2-(4-cyclopropylthiazol-2-yl)-7-methoxyquinolin-4-yloxy)-N-(cyclopropylsulfonyl)-6-(3,3-diethylureido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

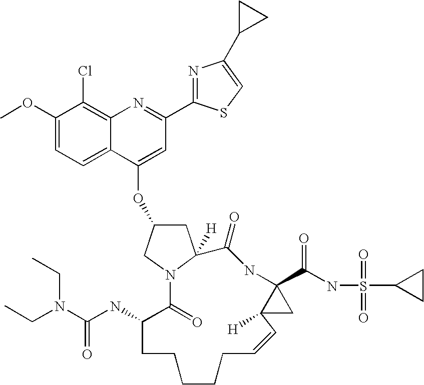

### 2-Bromo-1-cyclopropylethanone, intermediate 12

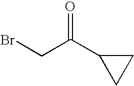

A solution of cyclopropyl methyl ketone (20.7 mL, 220 mmol) in MeOH (100 mL) was treated with bromine (11.3 mL, 220 mmol) at −5° C. for 2 hours. 50 mL water was added and the reaction was warmed up to room temperature overnight. The mixture was diluted with water (150 mL) and extracted with ethyl ether. The organic phases were separated and washed with NaHCO₃, dried (Na₂SO₄), and concentrated to dryness, affording a light yellow oil (35 g, 99%).

¹H NMR (CDCl₃) δ 0.91-1.02 (m, 2H), 1.03-1.16 (m, 2H), 2.08-2.20 (m, 1H), 4.02 (s, 2H).

### 4-Cyclopropyl-thiazole-2-carboxylic acid ethyl ester, intermediate 13

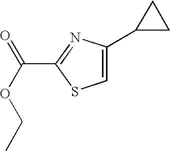

A solution of 2-bromo-1-cyclopropyl-ethanone (intermediate 12) (6.80 g, 42.0 mmol) and amino-thioxo-acetic acid ethyl ester (intermediate 6) (4.66 g, 35.0 mmol) in ethanol (50 mL) was heated to reflux for 1 hour. The reaction mixture was concentrated to dryness. The residue was purified by the flash column chromatography (silica, hexanes/ethyl acetate=4:1) to afford the title compound (6.7 g, 98%).

MS calcd for (C₉H₁₁NO₂S+H)⁺: 198.06

MS found: (M+H)⁺=198.05

### 4-Cyclopropyl-thiazole-2-carbonyl chloride, intermediate 14

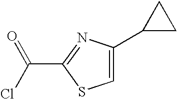

4-Cyclopropyl-thiazole-2-carboxylic acid ethyl ester (intermediate 13) (3.71 g, 18.8 mmol) was treated with NaOH (1.21 g, 30.1 mmol) in water (25 mL). The resulting mixture was stirred at room temperature overnight. The reaction was extracted with ethyl ether twice. The aqueous phase was adjusted pH to 2 by using 1N HCl and extracted with ethyl ether. The organic phase was separated, dried (Na₂SO₄), and concentrated. The residue was diluted with CH₂Cl₂ and oxayly chloride (3.28 mL, 37.6 mmol) was added and the reaction was stirred at room temperature for 1 hour. The mixture was concentrated to afford the title compound 14 as an oil (2.86 g, 80%).

### N-(6-acetyl-2-chloro-3-methoxyphenyl)-4-cyclopropylthiazole-2-carboxamide intermediate 15

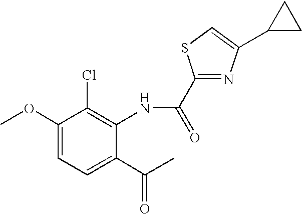

A solution of 1-(2-amino-3-chloro-4-methoxy-phenyl)-ethanone (intermediate 9) (3.32 g, 13.8 mmol) and pyridine (3.35 mL, 41.4 mmol) in CH₂Cl₂ (40 mL) was treated with 4-cyclopropyl-thiazole-2-carbonyl chloride (intermediate 14) at room temperature for 2 h. The mixture was diluted with CH₂Cl₂ and washed with brine. The organic phase was separated, dried (Na₂SO₄), and concentrated. The residue was purified by the flash column chromatography (silica, hexanes/ethyl acetate=1:1) to afford the title compound 15 (3.54 g, 60%).

MS calcd for (C₁₆H₁₅ClN₂O₃S+H)⁺: 351.05

MS found: (M+H)⁺=351.05

### 8-Chloro-2-(4-cyclopropylthiazol-2-yl)-7-methoxyquinolin-4-ol, intermediate 16

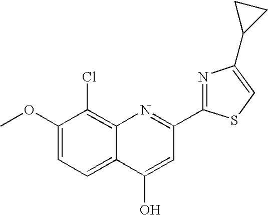

A solution of N-(6-acetyl-2-chloro-3-methoxyphenyl)-4-cyclopropylthiazole-2-carboxamide

(intermediate 15) (2.45 g, 7.00 mmol) in pyridine (30 mL) was treated with KOH (982 mg, 17.5 mmol) at room temperature and the reaction was heated to 110° C. for 2 h. The reaction mixture was concentrated to dryness. The residue was diluted with water and neutralized by acetic acid. The precipitate was filtered to afford the title compound 16 as a yellow solid (1.93 g, 83%).

MS calcd for (C₁₆H₁₃ClN₂O₂S+H)⁺: 333.05

MS found: (M+H)⁺=333.10

### 4,8-Dichloro-2-(4-cyclopropyl-thiazol-2-yl)-7-methoxy-quinoline, intermediate 17

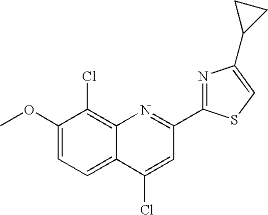

8-Chloro-2-(4-cyclopropyl-thiazol-2-yl)-7-methoxy-quinolin-4-ol (intermediate 16) (1.93 g, 5.81 mmol) and POCl₃ (5.32 mL, 58.1 mmol) was mixed at room temperature and the mixture was heated to 110° C. for 1 h. The mixture was concentrated to dryness. The residue was dissolved in CH₂Cl₂ and washed with NaHCO₃. The organic phase was separated, dried (Na₂SO₄), and concentrated to afford the title compound 17 (2.01 g, 99%).

MS calcd for (C₁₆H₁₂Cl₂N₂OS+H)⁺: 351.01

MS found: (M+H)⁺=350.7

### t-Butyl (2R,6S,13aS,14aR,16aS,Z)-2-(8-chloro-2-(4-cyclopropylthiazol-2-yl)-7-methoxyquinolin-4-yloxy)-14a-(cyclopropylsulfonylcarbamoyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-6-ylcarbamate, intermediate 18

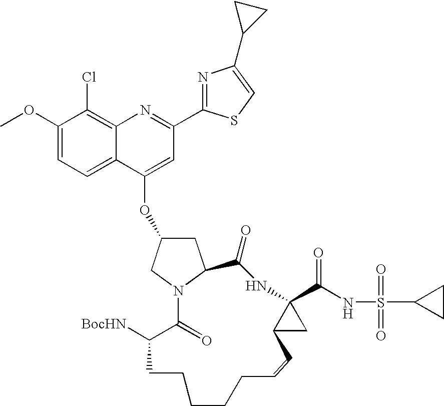

A solution of 4,8-dichloro-2-(4-cyclopropyl-thiazol-2-yl)-7-methoxy-quinoline (68 mg, 0.193 mmol) and t-butyl (2R,6S,13aS,14aR,16aS,Z)-14a-(cyclopropylsulfonylcarbamoyl)-2-hydroxy-5,16-dioxo-1,2,3,5,6,7,8,9,10,11,13a,14,14a,15,16,16a-hexadecahydrocyclopropa[e]pyrrolo[1,2-a][1,4]diazacyclopentadecin-6-ylcarbamate (100 mg, 0.176 mmol) in DMSO (3 mL) was treated with potassium t-butoxide (119 mg, 1.06 mmol) at room temperature overnight. The reaction was diluted with CH₂Cl₂ and washed with brine. The organic phase was separated, dried (Na₂SO₄), and concentrated. The residue was purified by preparative TLC (silica, hexanes/ethyl acetate=1:1), affording the title compound 18 (97 mg, 63%).

MS calcd for (C₄₂H₅₁ClN₆O₉S₂+H)⁺: 883.3

MS found: (M+H)⁺=883.1

### (2R,6S,13aS,14aR,16aS,Z)-6-amino-2-(8-chloro-2-(4-cyclopropylthiazol-2-yl)-7-methoxyquinolin-4-yloxy)-N-(cyclopropylsulfonyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide hydrochloride, intermediate 19

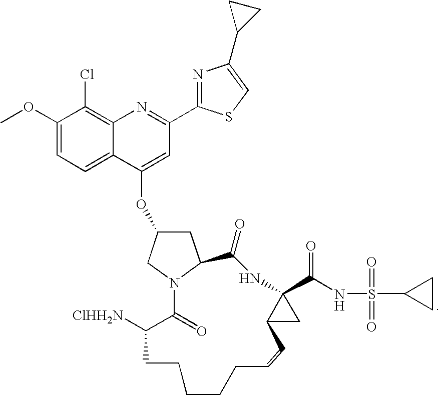

t-Butyl (2R,6S,13aS,14aR,16aS,Z)-2-(8-chloro-2-(4-cyclopropylthiazol-2-yl)-7-methoxyquinolin-4-yloxy)-14a-(cyclopropylsulfonylcarbamoyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-6-ylcarbamate (intermediate 18) (97 mg) was treated with HCl (0.5 mL, 4.0 M in dioxane) at room temperature overnight. The reaction was concentrated to afford the title compound 19 as a yellow solid (80 mg, 89%).

MS calcd for (C₃₇H₄₄Cl₂N₆O₇S₂+H−HCl)⁺: 783.2

MS found: (M+H)⁺=783.1

### (2R,6S,13aS,14aR,16aS,Z)-2-(8-chloro-2-(4-cyclopropylthiazol-2-yl)-7-methoxyquinolin-4-yloxy)-N-(cyclopropylsulfonyl)-6-(3,3-diethylureido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

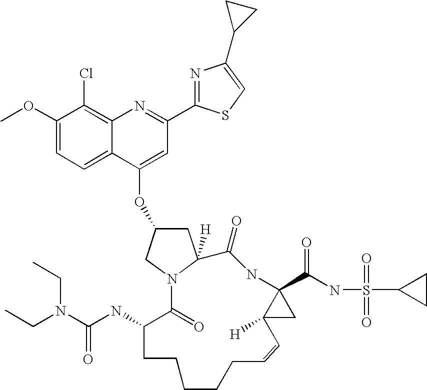

A solution of (2R,6S,13aS,14aR,16aS,Z)-6-amino-2-(8-chloro-2-(4-cyclopropylthiazol-2-yl)-7-methoxyquinolin-4-yloxy)-N-(cyclopropylsulfonyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide hydrochloride (intermediate 19) (80 mg, 0.10 mmol) and TEA (56 μL, 0.40 mmol) in THF (1 mL) was treated with diethylcarbamyl chloride (19 μL, 0.15 mmol) at room temperature overnight. The reaction was concentrated to dryness. The residue was purified by preparative TLC (silica, ethyl acetate) to yield the title compound example 4 (48 mg, 55%).

¹H NMR (CD₃OD) δ 0.84-1.14 (m, 16H), 1.27-1.62 (m, 12H), 1.79-1.92 (m, 2H), 2.16-2.22 (m, 1H), 2.31-2.37 (m, 1H), 2.64-2.76 (m, 2H), 2.92 (s, 1H), 3.14-3.27 (m, 4H), 4.06 (s, 3H), 4.18-4.21 (m, 1H), 4.46-4.48 (m, 1H), 4.66-4.69 (m, 1H), 5.07 (m, 1H), 5.52-5.59 (m, 1H), 5.71-5.74 (m, 1H), 7.13 (s, 1H), 7.29 (d, J=9.3 Hz, 1H), 7.65 (s, 1H), 8.19 (d, J=9.3 Hz, 1H).

MS calcd for (C₄₂H₅₂ClN₇O₈S₂+H)⁺: 882.3

MS found: (M+H)⁺=882.1

### Example 5

### (2R,6S,13aS,14aR,16aS)-N-(cyclopropylsulfonyl)-2-{[2-(4-cyclopropyl-1,3-thiazol-2-yl)-8-methyl-7-(methyloxy)-4-quinolinyl]oxy}-6-({[ethyl(1-methylethyl)amino]carbonyl}amino)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

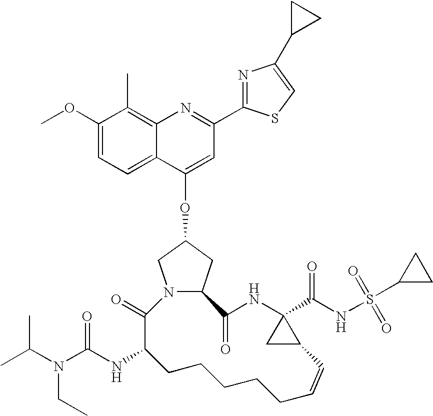

### ethyl(1-methylethyl)carbamic chloride

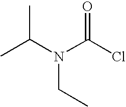

To a solution of ethylisopropylamine (500 mg, 5.7 mmol) in anhydrous THF (15 mL) cooled to 0° C. was added pyridine (0.92 mL, 11.4 mmol) followed by triphosgene (850 mg, 2.9 mmol) as a solution in anhydrous THF (10 mL) and the reaction was warmed to room temperature and stirred for 3.5 h. The reaction was concentrated in vacuo and the residue was dissolved in dichloromethane, washed with an equal volume of 0.1 N HCl. The organic layer was dried (MgSO₄) and concentrated in vacuo to afford the product as a brown oil (743 mg, 87% yield) which was carried on without further characterization.

### (2R,6S,13aS,14aR,16aS)-N-(cyclopropylsulfonyl)-2-{[2-(4-cyclopropyl-1,3-thiazol-2-yl)-8-methyl-7-(methyloxy)-4-quinolinyl]oxy}-6-({[ethyl(1-methylethyl)amino]carbonyl}amino)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

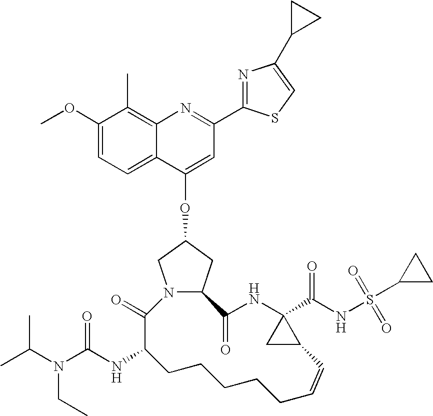

To a solution of (2R,6S,13aS,14aR,16aS)-6-amino-N-(cyclopropylsulfonyl)-2-{[2-(4-cyclopropyl-1,3-thiazol-2-yl)-8-methyl-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide hydrochloride intermediate 19 (60 mg, 0.07 mmol) and 4-dimethylaminopyridine (3 mg, 0.02 mmol) in anhydrous THF (1 mL) was added diisopropylethylamine (0.06 mL, 0.35 mmol) and ethyl(1-methylethyl)carbamic chloride (14 mg, 0.09 mmol) and the reaction was heated to 60° C. for 5 h. The reaction was concentrated in vacuo and purified by reverse phase C₁₈ HPLC eluting with 10-100% acetonitrile/water/0.1% formic acid to afford the title compound example 5 as an off-white solid (24.5 mg, 40% yield). 1H NMR (400 MHz, DMSO-d₆) d ppm 0.81-1.12 (m, 16H) 1.17-1.30 (m, 2H) 1.39 (br. s., 5H) 1.55 (d, J=8.79 Hz, 2H) 1.70 (br. s., 1H) 1.88 (br. s., 1H) 2.11-2.24 (m, 1H) 2.28-2.46 (m, 2H) 2.57 (s, 3H) 2.61-2.77 (m, 2H) 2.83-2.94 (m, 1H) 2.94-3.11 (m, 2H) 3.91 (d, J=2.74 Hz, 2H) 3.94 (s, 3H) 4.06 (quin, J=6.69 Hz, 1H) 4.18 (br. s., 1H) 4.39 (dd, J=9.87, 6.94 Hz, 1H) 4.69-4.91 (m, 1H) 5.05-5.20 (m, 1H) 5.62 (br. s., 2H) 6.11 (d, J=6.64 Hz, 1H) 7.26 (d, J=9.38 Hz, 1H) 7.42 (s, 1H) 7.50 (s, 1H) 8.14 (d, J=9.19 Hz, 1H) 8.81 (br. s., 1H) 11.11 (s, 1H). HRMS for C₄₄H₅₈N₇O₈S₂ (M+H)⁺ calc: 876.3788, found: 876.3783.

### Example 6

### (2R,6S,13aS,14aR,16aS)-N-(cyclopropylsulfonyl)-2-{[2-(4-cyclopropyl-1,3-thiazol-2-yl)-8-methyl-7-(methyloxy)-4-quinolinyl]oxy}-6-{[(dimethylamino)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

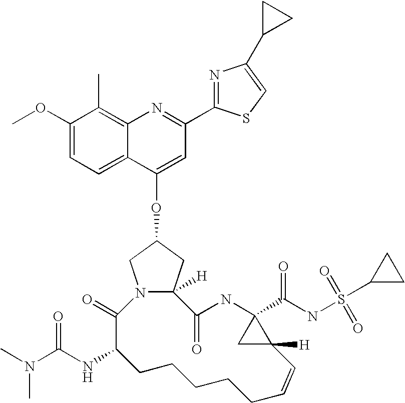

To a solution of (2R,6S,13aS,14aR,16aS)-6-amino-N-(cyclopropylsulfonyl)-2-{[2-(4-cyclopropyl-1,3-thiazol-2-yl)-8-methyl-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide hydrochloride intermediate 19 (75 mg, 0.09 mmol) and 4-dimethylaminopyridine (4 mg, 0.04 mmol) in anhydrous THF (1 mL) was added diisopropylethylamine (0.08 mL, 0.45 mmol) and dimethylcarbamoyl chloride (13 mg, 0.11 mmol) and the reaction was heated to 60° C. for 3 h. The reaction was concentrated in vacuo and purified by reverse phase C₁₈ HPLC eluting with 10-100% acetonitrile/water/0.1% formic acid to afford the title compound as an off-white solid (57 mg, 76% yield). 1H NMR (400 MHz, DMSO-d₆) d ppm 0.85-0.94 (m, 2H) 0.93-1.13 (m, 6H) 1.24 (br. s., 2H) 1.31-1.48 (m, 5H) 1.49-1.62 (m, 2H) 1.70 (br. s., 1H) 1.88 (br. s., 1H) 2.05-2.24 (m, 1H) 2.27-2.46 (m, 2H) 2.57 (s, 3H) 2.60-2.70 (m, 2H) 2.73 (s, 6H) 2.85-3.01 (m, 1H) 3.82-4.02 (m, 4H) 4.08-4.22 (m, 1H) 4.26-4.42 (m, 1H) 4.66-4.89 (m, 1H) 5.03-5.25 (m, 1H) 5.62 (br. s., 2H) 6.32 (br. s., 1H) 7.32 (d, J=9.19 Hz, 1H) 7.43 (s, 1H) 7.51 (s, 1H) 8.18 (d, J=9.19 Hz, 1H) 8.74 (br. s., 1H) 11.09 (br. s., 1H). HRMS for C₄₁H₅₂N₇O₈S₂ (M+H)⁺ calc: 834.3319, found: 834.3315.

### Example 7

### (2R,6S,13aS,14aR,16aS)-N-(cyclopropylsulfonyl)-2-{[2-(4-cyclopropyl-1,3-thiazol-2-yl)-8-methyl-7-(methyloxy)-4-quinolinyl]oxy}-6-({[methyl(1-methylethyl)amino]carbonyl}amino)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

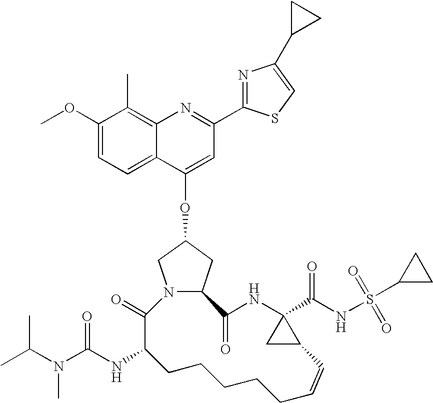

### methyl(1-methylethyl)carbamic chloride

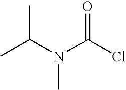

To a solution of methylisopropylamine (500 mg, 6.8 mmol) in anhydrous THF (20 mL) cooled to 0° C. was added pyridine (1.1 mL, 13.7 mmol) followed by triphosgene (1.01 g, 3.4 mmol) as a solution in anhydrous THF (15 mL) and the reaction was warmed to room temperature and stirred for 3.5 h. The reaction was concentrated in vacuo and the residue was dissolved in dichloromethane, washed with an equal volume of 0.1 N HCl. The organic layer was dried (MgSO₄) and concentrated in vacuo to afford the product as a yellow oil (881 mg, 96% yield) which was used without further characterization.

### (2R,6S,13aS,14aR,16aS)-N-(cyclopropylsulfonyl)-2-{[2-(4-cyclopropyl-1,3-thiazol-2-yl)-8-methyl-7-(methyloxy)-4-quinolinyl]oxy}-6-({[methyl(1-methylethyl)amino]carbonyl}amino)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

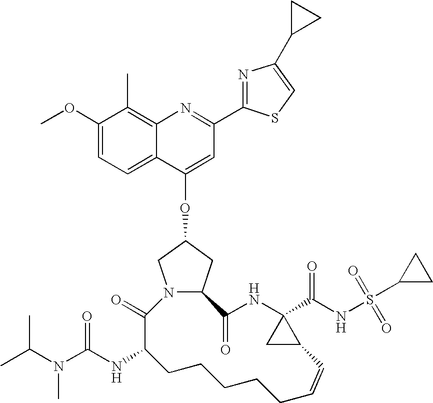

To a solution of (2R,6S,13aS,14aR,16aS)-6-amino-N-(cyclopropylsulfonyl)-2-{[2-(4-cyclopropyl-1,3-thiazol-2-yl)-8-methyl-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide hydrochloride (intermediate 19) (75 mg, 0.09 mmol) and 4-dimethylaminopyridine (4 mg, 0.04 mmol) in anhydrous THF (1 mL) was added diisopropylethylamine (0.08 mL, 0.45 mmol) and methyl(1-methylethyl)carbamic chloride (15 mg, 0.11 mmol) and the reaction was heated to 60° C. for 3 h. The reaction was concentrated in vacuo and purified by reverse phase C₁₈ HPLC eluting with 10-100% acetonitrile/water/0.1% formic acid to afford the title compound as an off-white solid (47 mg, 60% yield). 1H NMR (400 MHz, DMSO-d₆) d ppm 0.85-0.95 (m, 6H) 0.94-1.14 (m, 6H) 1.23 (br. s., 2H) 1.36 (br. s., 6H) 1.50-1.60 (m, 2H) 1.69 (br. s., 1H) 1.87 (br. s., 1H) 2.12-2.27 (m, 1H) 2.29-2.46 (m, 3H) 2.55 (s, 3H) 2.57 (s, 3H) 2.61-2.77 (m, 2H) 2.81-2.98 (m, 1H) 3.84-3.91 (m, 1H) 3.95 (s, 3H) 4.02-4.24 (m, 2H) 4.37 (dd, J=9.97, 6.84 Hz, 1H) 4.76-4.88 (m, 1H) 5.12 (t, J=9.19 Hz, 1H) 5.54-5.71 (m, 2H) 6.24 (d, J=6.64 Hz, 1H) 7.27 (d, J=9.38 Hz, 1H) 7.42 (s, 1H) 7.50 (s, 1H) 8.19 (d, J=9.19 Hz, 1H) 8.79 (s, 1H) 11.10 (s, 1H). HRMS for C₄₃H₆₆N₇O₈S₂ (M+H)⁺calc: 862.3632, found: 862.3625.

### Example 8

### (2R,6S,13aS,14aR,16aS)-6-({[bis(1-methylethyl)amino]carbonyl}amino)-N-(cyclopropylsulfonyl)-2-{[2-(4-cyclopropyl-1,3-thiazol-2-yl)-8-methyl-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

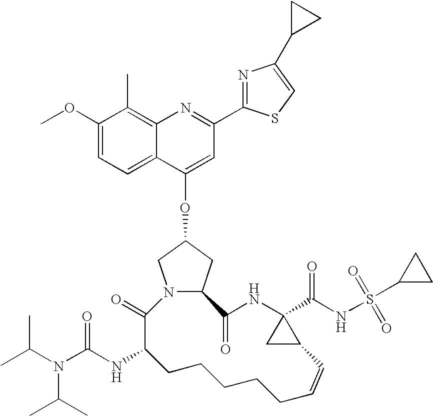

To a solution of (2R,68,13aS,14aR,16aS)-6-amino-N-(cyclopropylsulfonyl)-2-{[2-(4-cyclopropyl-1,3-thiazol-2-yl)-8-methyl-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide hydrochloride (intermediate 19) (75 mg, 0.09 mmol) and 4-dimethylaminopyridine (4 mg, 0.04 mmol) in anhydrous THF (1 mL) was added diisopropylethylamine (0.08 mL, 0.45 mmol) and diisopropylcarbamoyl chloride (19 mg, 0.11 mmol) and the reaction was heated to 60° C. for 3 h. The reaction was concentrated in vacuo and purified by reverse phase Cᵢg HPLC eluting with 10-100% acetonitrile/water/0.1% formic acid to afford the title compound as an off-white solid (35 mg, 44% yield). 1H NMR (400 MHz, DMSO-d₆) d ppm 0.79-1.14 (m, 16H) 1.14-1.31 (m, 3H) 1.38 (br. s., 6H) 1.56 (d, J=8.79 Hz, 2H) 1.71 (br. s., 1H) 1.83 (br. s., 1H) 2.14-2.27 (m, 1H) 2.28-2.47 (m, 3H) 2.57 (s, 3H) 2.61-2.77 (m, 2H) 2.90 (br. s., 1H) 3.49-3.68 (m, 2H) 3.73-3.99 (m, 5H) 4.11 (br. s., 1H) 4.42 (dd, J=10.16, 6.84 Hz, 1H) 4.80-4.94 (m, 1H) 5.04-5.19 (m, 1H) 5.62 (br. s., 2H) 6.00 (br. s., 1H) 7.26 (d, J=9.38 Hz, 1H) 7.41 (s, 1H) 7.49 (s, 1H) 8.07 (d, J=9.19 Hz, 1H) 8.87 (br. s., 1H) 11.11 (br. s., 1H). HRMS for C₄₆H₆₀N₇O₈S₂ (M+H)⁺ calc: 890.3945, found: 890.3939.

### Example 9

### (2R,6S,13aS,14aR,16aS)-N-(cyclopropylsulfonyl)-2-{[2-(4-cyclopropyl-1,3-thiazol-2-yl)-8-methyl-7-(methyloxy)-4-quinolinyl]oxy}-6-({[(1,1-dimethylethyl)(methyl)amino]carbonyl}amino)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

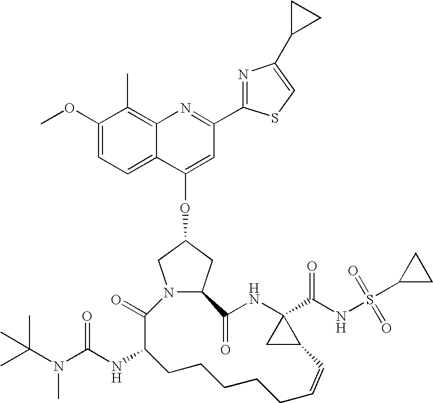

### (1,1-dimethylethyl)methylcarbamic chloride

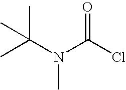

To a solution of tert-butylmethylamine (500 mg, 5.7 mmol) in anhydrous THF (15 mL) cooled to 0° C. was added pyridine (0.92 mL, 11.4 mmol) followed by triphosgene (850 mg, 2.9 mmol) as a solution in anhydrous THF (10 mL) and the reaction was warmed to room temperature and stirred for 3.5 h. The reaction was concentrated in vacuo and the residue was dissolved in dichloromethane, washed with an equal volume of 0.1 N HCl. The organic layer was dried (MgSO₄) and concentrated in vacuo to afford the product as a yellow solid (391 mg, 46% yield) which was used without further characterization.

### (2R,6S,13aS,14aR,16aS)-N-(cyclopropylsulfonyl)-2-{[2-(4-cyclopropyl-1,3-thiazol-2-yl)-8-methyl-7-(methyloxy)-4-quinolinyl]oxy}-6-({[(1,1-dimethylethyl)(methyl)amino]carbonyl}amino)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

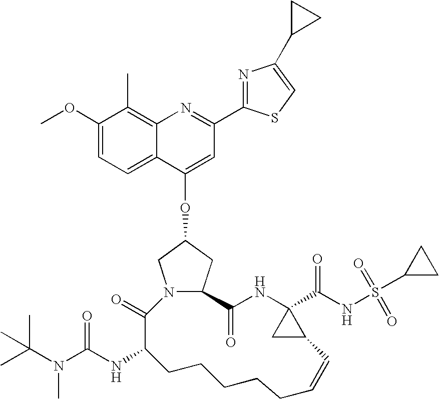

To a solution of (2R,6S,13aS,14aR,16aS)-6-amino-N-(cyclopropylsulfonyl)-2-{[2-(4-cyclopropyl-1,3-thiazol-2-yl)-8-methyl-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide hydrochloride (intermediate 19) (95 mg, 0.11 mmol) and 4-dimethylaminopyridine (13 mg, 0.11 mmol) in anhydrous THF (1 mL) was added diisopropylethylamine (0.115 mL, 0.66 mmol) and tert-butylmethylcarbamoyl chloride (25 mg, 0.17 mmol) and the reaction was heated to 60° C. for 3 h. The reaction was concentrated in vacuo and purified by reverse phase C₁₈ HPLC eluting with 10-100% acetonitrile/water/0.1% formic acid to afford the title compound as an off-white solid (7 mg, 7% yield). 1H NMR (400 MHz, DMSO-d₆) d ppm 0.79-1.14 (m, 10H) 1.14-1.29 (m, 6H) 1.39 (br. s., 6H) 1.50-1.63 (m, 2H) 1.70 (br. s., 1H) 1.77-1.90 (m, 1H) 2.12-2.28 (m, 1H) 2.26-2.46 (m, 3H) 2.57 (s, 3H) 2.67 (s, 4H) 2.91 (qd, 1H) 3.82-4.00 (m, 5H) 4.07 (br. s., 1H) 4.40 (dd, J=10.06, 6.74 Hz, 1H) 4.78-4.90 (m, 1H) 5.04-5.20 (m, 1H) 5.49-5.69 (m, 2H) 6.15-6.27 (m, 1H) 7.26 (d, J=9.19 Hz, 1H) 7.39-7.46 (m, 1H) 7.50 (s, 1H) 8.07 (d, J=9.19 Hz, 1H) 8.86 (br. s., 1H) 11.10 (s, 1H). HRMS for C₄₄H₅₈N₇O₈S₂ (M+H)⁺ calc: 876.3788, found: 876.3782.

### Example 10

### (2R,6S,13aS,14aR,16aS)-N-(cyclopropylsulfonyl)-6-{[(dimethylamino)carbonyl]amino}-2-{[6-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

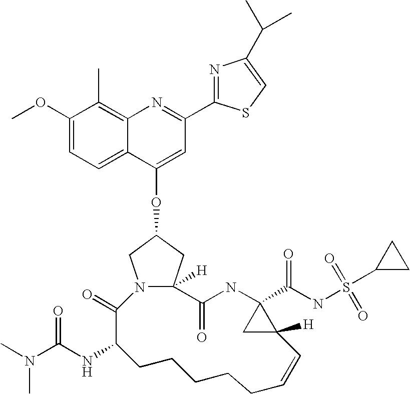

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.05 g, 0.06 mmol) in dichloroethane (3 mL) was added. N,N-diisopropylethylamine (0.077 g, 0.6 mmol) and triphosgene (0.018 g, 0.06 mmol). The reaction was stirred at rt for 1 h. Dimethyl amine (1 mL of a 2M sol) was added. The reaction was stirred for 16 h at rt. The reaction was concentrated in vacuo and purified by reverse phase O₁₈ HPLC eluting with 10-100% acetonitrile/water/0.1% formic acid to afford the title compound as an off-white solid (9 mg, 18% yield). 1H NMR (400 MHz, CHLOROFORM-d) δ ppm 0.89-0.99 (m, 1H) 1.05-1.19 (m, 2H) 1.29-1.37 (m, 2H) 1.40 (d, J=6.53 Hz, 6H) 1.44-1.55 (m, 5H) 1.62 (dd, J=9.29, 5.77 Hz, 2H) 1.84 (br. s., 1H) 1.89-1.95 (m, 2H) 2.24 (d, J=8.53 Hz, 1H) 2.64 (s, 3H) 2.69-2.76 (m, 2H) 2.86-2.94 (m, 6H) 3.16-3.28 (m, 1H) 3.49 (s, 1H) 3.74 (s, 1H) 3.88 (s, 2H) 4.05-4.13 (m, 1H) 4.41 (br. s., 1H) 4.55 (d, J=11.54 Hz, 1H) 4.63 (s, 1H) 4.88 (d, J=6.53 Hz, 1H) 5.03 (d, J=9.03 Hz, 1H) 5.51 (d, J=1.00 Hz, 1H) 5.75 (d, J=9.29 Hz, 1H) 7.04 (s, 1H) 7.08 (d, J=9.29 Hz, 1H) 7.47 (s, 1H) 7.94-8.03 (m, 2H) 10.46 (br. s., 1H). LCMS 836 (M+H).

### Example 11

### (2R,6S,13aS,14aR,16aS)-N-(cyclopropylsulfonyl)-6-({[(2R,6S)-2,6-dimethyl-4-morpholinyl]carbonyl}amino)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

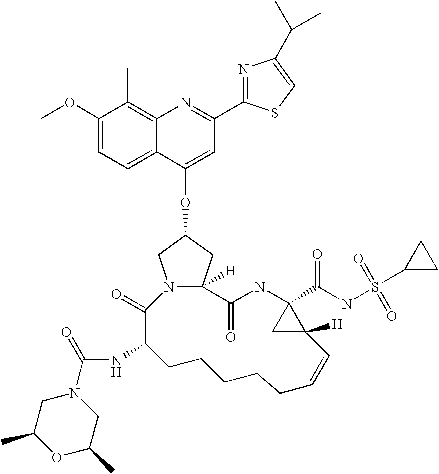

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.05 g, 0.06 mmol) in dichloroethane (3 mL) was added N,N-diisopropylethylamine (0.077 g, 0.6 mmol) and triphosgene (0.018 g, 0.06 mmol). The reaction was stirred at rt for 1 h. c is 2,6-Dimethylmorpholine (0.036 g, 0.32 mmol) was added. The reaction was stirred for 16 h at rt. The reaction was concentrated in vacuo and purified by reverse phase C₁₈ HPLC eluting with 10-100% acetonitrile/water/0.1% formic acid to afford the title compound as an off-white solid (18 mg, 31% yield). 1H NMR (400 MHz, CHLOROFORM-d) δ ppm 0.88-1.00 (m, 1H) 1.17 (dd, J=6.15, 3.89 Hz, 4H) 1.39-1.46 (m, 6H) 1.46-1.57 (m, 6H) 1.87-2.01 (m, 3H) 2.27 (d, J=8.53 Hz, 1H) 2.37-2.54 (m, 3H) 2.64 (s, 6H) 2.69 (s, 3H) 2.75 (d, J=7.03 Hz, 2H) 2.87-2.97 (m, 1H) 3.20-3.30 (m, 1H) 3.46-3.58 (m, 2H) 3.62 (d, J=13.30 Hz, 1H) 3.97 (s, 3H) 4.07-4.16 (m, 1H) 4.46 (br. s., 1H) 4.63 (s, 1H) 4.68-4.75 (m, 1H) 4.97 (d, J=7.53 Hz, 1H) 5.02 (d, J=9.54 Hz, 1H) 5.56 (br. s., 1H) 5.75 (d, J=9.29 Hz, 1H) 6.79-6.90 (m, 1H) 7.05 (s, 1H) 7.15 (d, J=9.03 Hz, 1H) 7.53-7.60 (m, 1H) 8.03 (s, 1H) 8.07 (d, J=9.29 Hz, 1H) 10.25 (br. s., 1H). LCMS 906 (M+H).

### Example 12

### (2R,6S,13aS,14aR,16aS)-N-(cyclopropylsulfonyl)-6-{[(1,1-dioxido-4-thiomorpholinyl)carbonyl]amino}-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

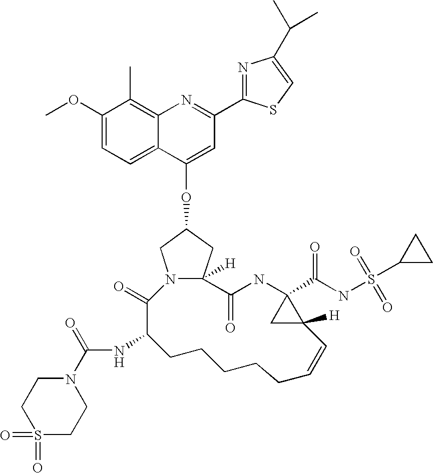

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.05 g, 0.06 mmol) in dichloroethane (3 mL) was added N,N-diisopropylethylamine (0.077 g, 0.6 mmol) and triphosgene (0.018 g, 0.06 mmol). The reaction was stirred at rt for 1 h. Thiomorpholine 1,1-dioxide (0.043 g, 0.32 mmol) was added. The reaction was stirred for 1 h at rt. The reaction was concentrated in vacuo and purified by reverse phase C₁₈ HPLC eluting with 10-100% acetonitrile/water/0.1% formic acid to afford the title compound as an off-white solid (32 mg, 55% yield). 1H NMR (400 MHz, CHLOROFORM-d) δ ppm 0.88-1.04 (m, 1H) 1.07-1.24 (m, 2H) 1.31 (d, J=4.52 Hz, 2H) 1.38-1.47 (m, 5H) 1.46-1.58 (m, 5H) 1.58-1.73 (m, 2H) 1.75-1.86 (m, 2H) 1.97 (d, J=1.25 Hz, 2H) 2.23 (d, 1H) 2.53-2.65 (m, 1H) 2.68 (s, 3H) 2.72-2.97 (m, 8H) 3.19-3.33 (m, 1H) 3.64 (br. s., 3H) 3.97 (s, 3H) 4.02-4.14 (m, 1H) 4.31-4.40 (m, 1H) 4.61-4.74 (m, 2H) 5.03 (br. s., 1H) 5.32 (br. s., 1H) 5.57 (br. s., 1H) 5.74 (br. s., 1H) 6.77-6.90 (m, 1H) 7.07 (br. s., 1H) 7.18 (d, J=9.29 Hz, 1H) 7.47-7.60 (m, 1H) 7.95 (d, J=8.53 Hz, 1H) 8.01 (s, 1H) 10.18-10.27 (m, 1H). LCMS 926 (M+H).

### Example 13

### (2R,6S,13aS,14aR,16aS)-6-{[(4-cyclopentyl-1-piperazinyl)carbonyl]amino}-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

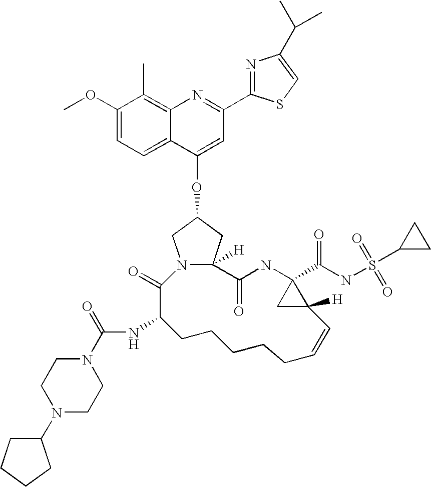

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.05 g, 0.06 mmol) in dichloroethane (3 mL) was added N,N-diisopropylethylamine (0.077 g, 0.6 mmol) and triphosgene (0.018 g, 0.06 mmol). The reaction was stirred at rt for 1 h. 1-Cyclopentylpiperazine (0.049 g, 0.32 mmol) was added. The reaction was stirred for 1 h at rt. The reaction was concentrated in vacuo and purified by reverse phase C₁₈ HPLC eluting with 10-100% acetonitrile/water/0.1% formic acid to afford the title compound as an off-white solid (6 mg, 10% yield). 1H NMR (400 MHz, CHLOROFORM-c/) δ ppm 0.86-0.99 (m, 1H) 1.04-1.21 (m, 2H) 1.26 (d, J=6.02 Hz, 2H) 1.40 (d, J=7.03 Hz, 6H) 1.42-1.55 (m, 8H) 1.57-1.77 (m, 6H) 1.77-1.95 (m, 6H) 2.23 (q, J=8.45 Hz, 1H) 2.53-2.63 (m, 1H) 2.66 (s, 4H) 2.68-2.83 (m, 5H) 2.85-2.96 (m, 1H) 3.22 (dq, J=7.03, 6.86 Hz, 1H) 3.46 (br. s., 4H) 3.93 (s, 3H) 4.08 (dd, J=11.17, 3.64 Hz, 1H) 4.32-4.42 (m, 1H) 4.61-4.72 (m, 1H) 4.98 (t, J=9.41 Hz, 1H) 5.51 (br. s., 2H) 5.69 (q, J=8.95 Hz, 1H) 7.03 (s, 1H) 7.12 (d, J=9.03 Hz, 1H) 7.48 (s, 1H) 7.62 (br. s., 1H) 8.02 (d, J=9.03 Hz, 1H) 8.30 (s, 1H). LCMS 945 (M+H).

### Example 14

### (2R,6S,13aS,14aR,16aS)-N-(cyclopropylsulfonyl)-2-[{4-(1-methyl-2-[4-(1-methylethyl)-1,3-thiazol-2-yl]-7-(methyloxy)-4-quinolinyl]oxy}-6-[(4-morpholinylcarbonyl)amino]-5,16-dioxo-1,2,3,6,7,8,9,10,11,13a,14,15,16,16a-tetradecahydrocyclocropa[e]pyrrolo[1,2-a][1,4]diazacyclopentadecine-14a(5H)-carboxamide

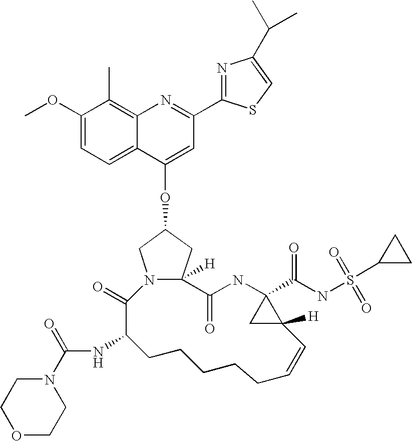

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.066 g, 0.08 mmol) in dichloroethane (3 mL) was added N,N-diisopropylethylamine (0.02 g, 0.16 mmol) and triphosgene (0.023 g, 0.08 mmol). The reaction was stirred at it for 1 h. Morpholine (0.026 g, 0.30 mmol) was added. The reaction was stirred for 1 h at rt. The reaction was concentrated in vacuo and purified by reverse phase C₁₈ HPLC eluting with 10-100% acetonitrile/water/0.1% formic acid to afford the title compound as an off-white solid (39 mg, 52% yield). 1H NMR (400 MHz, CHLOROFORM-4) δ ppm 0.89-1.00 (m, 1H) 1.10 (br. s., 2H) 1.27-1.36 (m, 2H) 1.41 (d, J=7.03 Hz, 6H) 1.44-1.55 (m, 6H) 1.62 (ddd, J=7.22, 4.96, 2.64 Hz, 3H) 1.92-1.99 (m, 2H) 2.25 (s, 1H) 2.20-2.31 (m, 1H) 2.52-2.63 (m, 1H) 2.69 (s, 3H) 2.71-2.77 (m, 2H) 2.92 (br. s., 1H) 3.17-3.35 (m, 4H) 3.63 (d, J=4.27 Hz, 4H) 3.97 (s, 3H) 4.07-4.18 (m, 1H) 4.41-4.51 (m, 1H) 4.62 (t, J=7.28 Hz, 2H) 4.93 (br. s., 1H) 5.03 (br. s., 1H) 5.57 (d, J=1.25 Hz, 1H) 5.69-5.81 (m, 1H) 6.75-6.84 (m, 1H) 7.06 (s, 1H) 7.15 (d, J=9.03 Hz, 1H) 7.56 (br. s., 1H) 8.03 (d, J=9.04 Hz, 1H) 10.20-10.30 (m, 1H). LCMS 878 (M+H).

### Example 15

### (2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-[(1-pyrrolidinylcarbonyl)amino]-1,2,3,6,7,8,9,10,11,13a,14,15,16,16a-tetradecahydrocyclopropa[e]pyrrolo[1,2-a][1,4]diazacyclopentadecine-14a(5H)-carboxamide

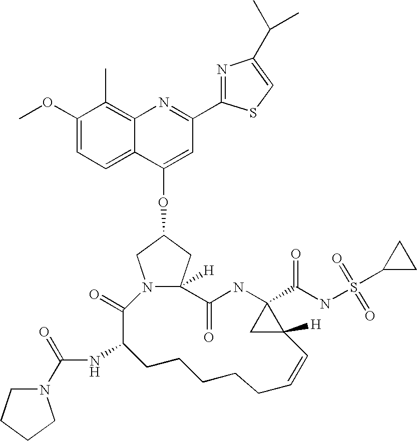

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.062 g, 0.07 mmol) in dichloroethane (3 mL) was added N,N-diisopropylethylamine (0.019 g, 0.15 mmol) and triphosgene (0.022 g, 0.07 mmol). The reaction was stirred at rt for 1 h. Pyrrolidine (0.021 g, 0.30 mmol) was added. The reaction was stirred for 1 h at rt. concentrated in vacuo and purified by reverse phase C₁₈ HPLC eluting with 10-100% acetonitrile/water/0.1% formic acid to afford the title compound as an off-white solid (31 mg, 44% yield). 1H NMR (400 MHz, CHLOROFORM-d) δ ppm 0.87-1.01 (m, 1H) 1.06-1.24 (m, 2H) 1.29-1.38 (m, 2H) 1.42 (d, J=6.78 Hz, 6H) 1.45-1.60 (m, 6H) 1.66-1.77 (m, 1H) 1.82-1.98 (m, 6H) 2.27 (d, J=9.03 Hz, 1H) 2.52-2.63 (m, 1H) 2.67 (s, 3H) 2.73 (br. s., 2H) 2.92 (s, 1H) 3.30 (d, J=6.27 Hz, 4H) 3.23-3.35 (m, 3H) 3.93 (s, 3H) 4.09-4.23 (m, 1H) 4.47-4.56 (m, 1H) 4.62 (br. s., 2H) 4.66-4.74 (m, 1H) 4.97-5.07 (m, 1H) 5.54-5.61 (m, 1H) 5.69-5.83 (m, 1H) 7.06 (s, 1H) 7.09-7.17 (m, 1H) 7.61 (br. s., 1H) 8.05 (d, J=8.78 Hz, 1H) 10.30-10.42 (m, 1H). LCMS 862 (M+H).

### Example 16

### (2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-[(1-piperidinylcarbonyl)amino]-1,2,3,6,7,8,9,10,11,13a,14,15,16,16a-tetradecahydrocyclopropa[e]pyrrolo[1,2-a][1,4]diazacyclopentadecine-14a(5H)-carboxamide

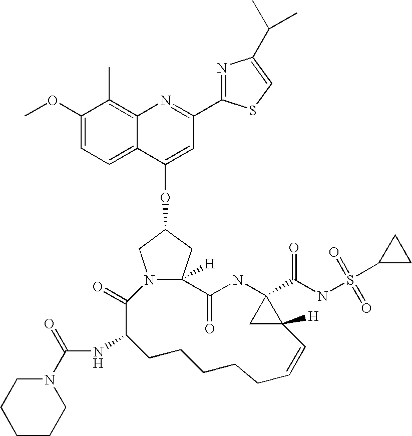

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.062 g, 0.07 mmol) in dichloroethane (3 mL) was added N,N-diisopropylethylamine (0.019 g, 0.15 mmol) and triphosgene (0.022 g, 0.07 mmol). The reaction was stirred at rt for 1 h. Piperidine (0.025 g, 0.30 mmol) was added. The reaction was stirred for 1 h at rt. concentrated in vacuo and purified by reverse phase C₁₈ HPLC eluting with 10-100% acetonitrile/water/0.1% formic acid to afford the title compound as an off-white solid (37 mg, 54% yield). 1H NMR (400 MHz, CHLOROFORM-d) δ ppm 0.85-1.00 (m, 1H) 1.04-1.22 (m, 2H) 1.33 (br. s., 2H) 1.41 (d, J=7.03 Hz, 6H) 1.45-1.71 (m, 15H) 1.91-1.99 (m, 2H) 2.25 (d, J=8.53 Hz, 1H) 2.54-2.63 (m, 1H) 2.67 (s, 3H) 2.70-2.77 (m, 2H) 2.92 (s, 1H) 3.18-3.28 (m, 2H) 3.32 (d, J=4.77 Hz, 2H) 3.93 (s, 3H) 4.07-4.17 (m, 1H) 4.38-4.51 (m, 1H) 4.62 (br. s., 2H) 4.88 (br. s., 1H) 4.98-5.09 (m, 1H) 5.55 (br. s., 1H) 5.69-5.82 (m, 1H) 6.90-7.02 (m, 1H) 7.05 (s, 1H) 7.09-7.15 (m, 1H) 7.48-7.61 (m, 1H) 8.08 (d, J=9.03 Hz, 1H) 10.29-10.37 (m, 1H). LCMS 876 (M+H).

### Example 17

### (2R,6S,13aS,14aR,16aS)-N-(cyclopropylsulfonyl)-6-({([ethyl(methyl)amino]carbonyl}amino)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

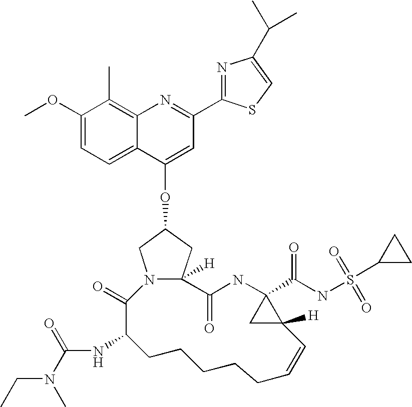

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.048 g, 0.06 mmol) in dichloroethane (3 mL) was added triethylamine (0.018 g, 0.18 mmol) and triphosgene (0.020 g, 0.06 mmol). The reaction was stirred at rt for 1 h then was added to a solution of ethylamine (0.054 g, 0.30 mmol). The reaction was stirred for 16 h at rt. concentrated in vacuo and purified by reverse phase C₁₈ HPLC eluting with 10-100% acetonitrile/water/0.1% formic acid to afford the title compound as an off-white solid (5 mg, 10% yield). 1H NMR (400 MHz, CHLOROFORM-d) δ ppm 0.87-1.00 (m, 2H) 1.05-1.13 (m, 3H) 1.11-1.23 (m, 3H) 1.40 (d, J=7.03 Hz, 6H) 1.44-1.58 (m, 6H) 1.61-1.72 (m, 2H) 1.89-1.96 (m, 2H) 2.20-2.33 (m, 2H) 2.55-2.64 (m, 1H) 2.68 (s, 3H) 2.70-2.76 (m, 2H) 2.84 (s, 3H) 2.88-2.96 (m, 1H) 3.18-3.34 (m, 3H) 3.94 (s, 3H) 3.95-4.02 (m, 1H) 4.05-4.13 (m, 1H) 4.42-4.53 (m, 1H) 4.57-4.72 (m, 2H) 4.80-4.92 (m, 1H) 4.96-5.09 (m, 1H) 5.51-5.59 (m, 1H) 5.68-5.83 (m, 1H) 7.02-7.05 (m, 1H) 7.10-7.16 (m, 1H) 7.49-7.51 (m, 1H) 8.03-8.09 (m, 1H). LCMS 850 (M+H)

### Example 18

### (2R,6S,13aS,14aR,16aS)-6-[(1-azetidinylcarbonyl)amino]-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

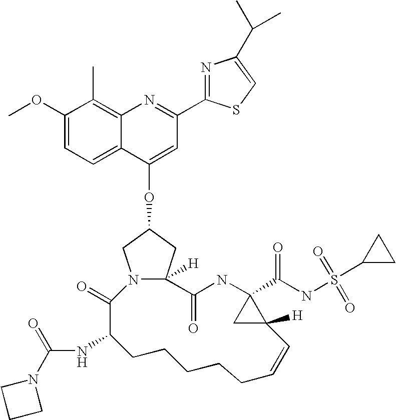

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.075 g, 0.09 mmol) in dichloroethane (3 mL) was added triethylamine (0.029 g, 0.28 mmol) and triphosgene (0.018 g, 0.06 mmol). The reaction was stirred at it for 1 h then was added to a solution of ethylamine (0.054 g, 0.30 mmol). The reaction was stirred for 16 h at rt. concentrated in vacuo and purified by reverse phase C₁₈ HPLC eluting with 10-100% acetonitrile/water/0.1% formic acid to afford the title compound as an off-white solid (4 mg, 5% yield). 1H NMR (400 MHz, CHLOROFORM-d) δ ppm 0.82-1.02 (m, 1H) 1.06-1.22 (m, 2H) 1.24-1.37 (m, 5H) 1.40 (d, J=6.78 Hz, 6H) 1.45-1.55 (m, 6H) 1.91-1.98 (m, 2H) 2.20-2.32 (m, 2H) 2.52-2.62 (m, 1H) 2.69 (s, 3H) 2.71-2.77 (m, 2H) 2.87-2.95 (m, 2H) 3.08-3.28 (m, 3H) 3.90-3.96 (m, 2H) 3.97 (s, 3H) 4.08-4.17 (m, 1H) 4.46-4.66 (m, 2H) 4.70-4.76 (m, 1H) 4.97-5.06 (m, 1H) 5.51-5.58 (m, 1H) 5.69-5.80 (m, 1H) 6.90 (br. s., 1H) 7.04 (br. s., 1H) 7.15-7.19 (m, 1H) 7.52 (s, 1H) 8.00-8.05 (m, 1H) 8.10 (br. s., 1H). LCMS 848 (M+H).

### Example 19

### (2R,6S,13aS,14aR,16aS)-6-({[(cyclopropylmethyl)(propyl)amino]carbonyl}amino)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

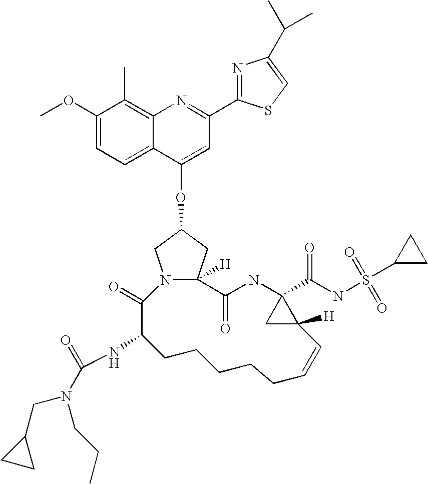

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.050 g, 0.06 mmol) in dichloroethane (3 mL) was added N,N-diisopropylethylamine (0.015 g, 0.12 mmol) and triphosgene (0.018 g, 0.06 mmol). The reaction was stirred at rt for 1 h. (Cyclopropylmethyl)propylamine (0.034 g, 0.30 mmol) was added. The reaction was stirred for 1 h at rt. The reaction was concentrated in vacuo and purified by reverse phase C₁₈ HPLC eluting with 10-100% acetonitrile/water/0.1% formic acid to afford the title compound as an off-white solid (14 mg, 26% yield). 1H NMR (400 MHz, CHLOROFORM-d) δ ppm 0.15 (q, J=4.88 Hz, 2H) 0.44-0.51 (m, 2H) 0.80-0.94 (m, 4H) 1.00-1.17 (m, 2H) 1.22-1.32 (m, 2H) 1.33-1.39 (m, 6H) 1.40-1.56 (m, 6H) 1.63 (d, J=2.75 Hz, 4H) 1.74-1.94 (m, 3H) 2.21 (t, J=8.61 Hz, 1H) 2.52-2.60 (m, 1H) 2.63-2.66 (m, 3H) 2.69 (dd, J=7.87, 2.93 Hz, 2H) 2.80-2.92 (m, 1H) 3.03 (d, J=6.41 Hz, 1H) 3.09-3.22 (m, 4H) 3.90 (s, 3H) 4.04 (dd, J=11.26, 3.94 Hz, 1H) 4.45 (ddd, J=10.48, 7.55, 3.02 Hz, 1H) 4.56 (t, J=7.87 Hz, 1H) 4.68 (d, J=11.35 Hz, 1H) 4.85 (d, J=7.51 Hz, 1H) 4.93-5.03 (m, 1H) 5.48 (br. s., 1H) 5.64-5.78 (m, 1H) 6.84 (br. s., 1H) 6.99 (s, 1H) 7.10 (d, J=9.16 Hz, 1H) 7.46 (s, 1H) 8.02 (d, J=9.16 Hz, 1H) 10.24 (br. s., 1H). LCMS 904 (M+H).

### Example 20

### (2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-{[(4-phenyl-1-piperidinyl)carbonyl]amino}-1,2,3,6,7,8,9,10,11,13a,14,15,16,16a-tetradecahydrocyclopropa[e]pyrrolo[1,2-a][1,4]diazacyclopentadecine-14a(5H)-carboxamide

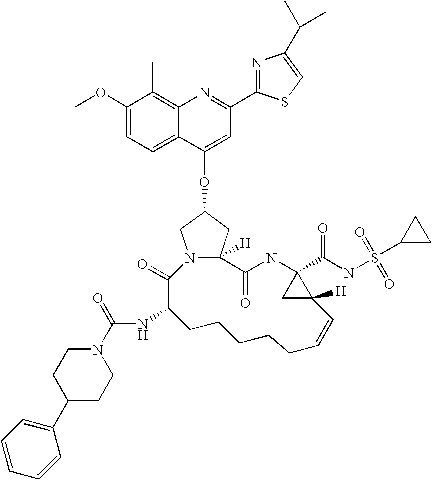

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.052 g, 0.06 mmol) in dichloroethane (3 mL) was added N,N-diisopropylethylamine (0.015 g, 0.12 mmol) and triphosgene (0.018 g, 0.06 mmol). The reaction was stirred at rt for 1 h. 4-Phenylpiperidine (0.048 g, 0.30 mmol) was added. The reaction was stirred for 1 h at rt. The reaction was concentrated in vacuo and purified by reverse phase C₁₈ HPLC eluting with 10-100% acetonitrile/water/0.1% formic acid to afford the title compound as an off-white solid (12 mg, 20% yield). 1H NMR (400 MHz, CHLOROFORM-d) δ ppm 0.90 (s, 2H) 1.05-1.24 (m, 2H) 1.26-1.38 (m, 2H) 1.41 (d, J=6.78 Hz, 6H) 1.44-1.76 (m, 7H) 1.80-1.89 (m, 2H) 1.95 (d, J=6.53 Hz, 2H) 2.21-2.32 (m, 1H) 2.56-2.65 (m, 1H) 2.67 (s, 3H) 2.72-2.99 (m, 6H) 3.19-3.32 (m, 1H) 3.89 (s, 3H) 3.91-3.96 (m, 1H) 4.04-4.19 (m, 3H) 4.40-4.50 (m, 1H) 4.65 (d, J=7.53 Hz, 2H) 4.94-5.00 (m, 1H) 5.04 (s, 1H) 5.52-5.61 (m, 1H) 5.67-5.83 (m, 1H) 7.05 (s, 1H) 7.12 (d, J=9.29 Hz, 1H) 7.17-7.21 (m, 3H) 7.23 (d, J=7.28 Hz, 1H) 7.29-7.34 (m, 3H) 7.52-7.61 (m, 1H) 8.09 (d, J=9.03 Hz, 1H) 10.29-10.45 (m, 1H). LCMS 904 (M+H).

### Example 21

### (2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-({[4-(1-pyrrolidinyl)-1-piperidinyl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide

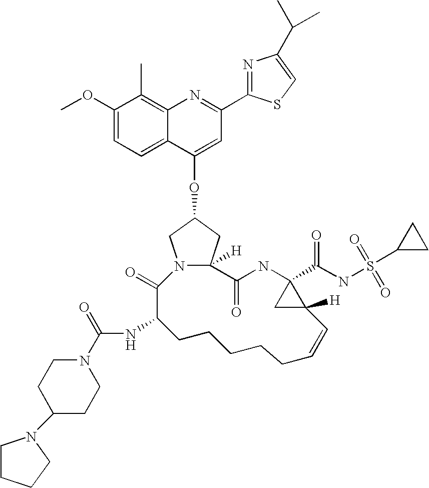

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.050 g, 0.06 mmol) in dichloroethane (3 mL) was added N,N-diisopropylethylamine (0.015 g, 0.12 mmol) and triphosgene (0.018 g, 0.06 mmol). The reaction was stirred at rt for 1 h. 4-(1-Pyrrolidinyl)piperidine (0.046 g, 0.30 mmol) was added. The reaction was stirred for 1 h at rt. The reaction was concentrated in vacuo and purified by reverse phase C₁₈ HPLC eluting with 10-100% acetonitrile/water/0.1% formic acid to afford the title compound as an off-white solid (12 mg, 20% yield). 1H NMR (400 MHz, CHLOROFORM-d) δ ppm 0.88-0.99 (m, 1H) 1.04-1.24 (m, 2H) 1.33 (br. s., 2H) 1.41 (d, J=7.03 Hz, 6H) 1.44-1.65 (m, 17H) 1.77-1.87 (m, 2H) 1.90-1.99 (m, 2H) 2.25 (d, J=8.53 Hz, 2H) 2.55-2.63 (m, 1H) 2.67 (s, 3H) 2.71-2.79 (m, 2H) 2.92 (s, 1H) 3.18-3.28 (m, 3H) 3.32 (d, J=4.77 Hz, 3H) 3.93 (s, 3H) 4.06-4.19 (m, 1H) 4.39-4.49 (m, 1H) 4.58-4.71 (m, 2H) 4.88 (br. s., 1H) 4.99-5.08 (m, 1H) 5.55 (br. s., 1H) 5.69-5.83 (m, 1H) 6.90-7.01 (m, 1H) 7.05 (s, 1H) 7.09-7.18 (m, 1H) 7.54 (br. s., 1H) 8.08 (d, J=9.03 Hz, 1H) 10.25-10.40 (m, 1H). LCMS 945 (M+H).

### Example 22

### (2R,6S,13aS,14aR,16aS)-N-(cyclopropylsulfonyl)-6-{[(4-hydroxy-1-piperidinyl)carbonyl]amino}-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16 ,16a-tetradecahydrocyclopropa[e]pyrrolo[1,2-a][1,4]diazacyclopentadecine-14a(5H)-carboxamide

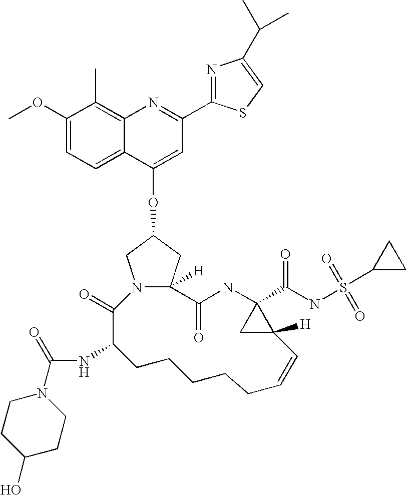

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.059 g, 0.07 mmol) in dichloroethane (3 mL) was added N,N-diisopropylethylamine (0.018 g, 0.14 mmol) and triphosgene (0.021 g, 0.07 mmol). The reaction was stirred at rt for 1 h. 4-Piperidinol (0.030 g, 0.30 mmol) was added. The reaction was stirred for 1 h at rt. The reaction was concentrated in vacuo and purified by reverse phase C₁₈ HPLC eluting with 10-100% acetonitrile/water/0.1% formic acid to afford the title compound as an off-white solid (18 mg, 28% yield). 1H NMR (400 MHz, CHLOROFORM-d) δ ppm 0.89-1.00 (m, 1H) 1.05-1.21 (m, 2H) 1.30-1.37 (m, 2H) 1.41 (d, J=6.78 Hz, 6H) 1.44-1.55 (m, 6H) 1.55-1.72 (m, 4H) 1.79-1.89 (m, 4H) 1.92 (dd, J=7.53, 6.27 Hz, 2H) 2.17-2.28 (m, 1H) 2.65 (br. s., 4H) 2.70-2.77 (m, 2H) 2.85-3.03 (m, 2H) 3.04-3.15 (m, 1H) 3.17-3.30 (m, 1H) 3.55-3.67 (m, 1H) 3.75 (s, 1H) 3.85-3.91 (m, 3H) 4.05-4.09 (m, 1H) 4.40 (br. s., 1H) 4.54-4.68 (m, 2H) 4.93 (d, J=6.02 Hz, 1H) 4.96-5.06 (m, 1H) 5.53 (br. s., 1H) 5.70-5.82 (m, 1H) 7.05 (s, 1H) 7.08 (d, J=8.78 Hz, 1H) 7.31-7.43 (m, 1H) 7.44-7.56 (m, 1H) 8.02 (d, J=9.29 Hz, 1H) 10.44 (br. s., 1H). LCMS 892 (M+H)

### Example 23

### (2R,6S,13aS,14aR,16aS)-6-({[4-(2-amino-2-oxoethyl)-1-piperidinyl]carbonyl}amino)-N-(cyclopropylsulfonyl)2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide.

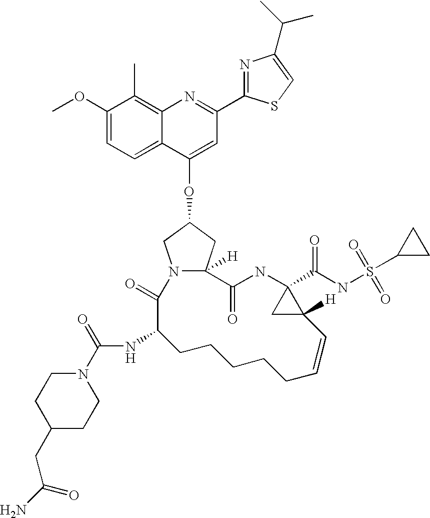

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (0.062 g, 0.07 mmol) in dichloroethane (3 mL) was added. N,N-diisopropylethylamine (0.019 g, 0.14 mmol) and triphosgene (0.022 g, 0.07 mmol). The reaction was stirred at it for 1 h. 2-(4-Piperidinyl)acetamide (0.042 g, 0.30 mmol) was added. The reaction was stirred for 1 h at rt. The reaction was concentrated in vacuo and purified by reverse phase C₁₈ HPLC eluting with 10-100% acetonitrile/water/0.1% formic acid to afford title compound 1 as an off-white solid (6 mg, 9% yield) and title compound 2 as an off white solid (9 mg, 14%). 1H NMR (400 MHz, CHLOROFORM-d) δ ppm 0.89-1.02 (m, 1H) 1.08-1.22 (m, 2H) 1.24-1.38 (m, 2H) 1.38-1.45 (m, 6H) 1.45-1.57 (m, 6H) 1.58-1.88 (m, 6H) 1.87-2.02 (m, 2H) 2.14 (d, J=7.03 Hz, 2H) 2.20-2.34 (m, 1H) 2.62 (br. s., 3H) 2.66-2.84 (m, 2H) 2.92 (br. s., 1H) 3.18-3.31 (m, 1H) 3.86 (br. s., 4H) 4.06-4.17 (m, 1H) 4.32-4.44 (m, 1H) 4.55-4.73 (m, 2H) 4.90-5.10 (m, 3H) 4.90-5.09 (m, 1H) 5.47-5.60 (m, 1H) 5.53 (br. s., 1H) 5.68-5.89 (m, 1H) 5.69-5.87 (m, 1H) 7.01-7.11 (m, 2H) 7.06 (br. s., 1H) 7.40-7.63 (m, 1H) 7.43-7.61 (m, 1H) 7.95-8.09 (m, 1H) 7.98-8.07 (m, 1H) 10.49-10.60 (m, 1H) 10.49-10.60 (m, 1H). LCMS 932 (M+H).

### Example 24

### (2R,6S,13aS,14aR,16aS)-N-(cyclopropylsulfonyl)-6-[(hexahydrocyclopenta[c]pyrrol-2(1H)-ylcarbonyl)amino]-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

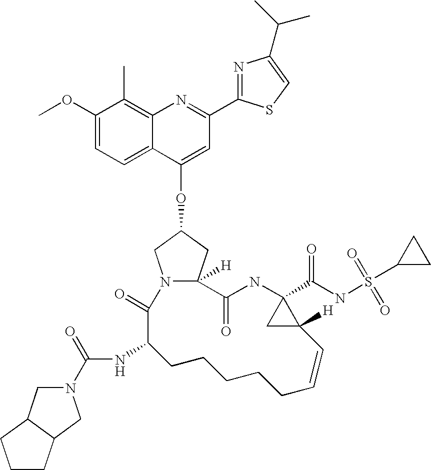

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.059 g, 0.07 mmol) in dichloroethane (3 mL) was added N,N-diisopropylethylamine (0.018 g, 0.14 mmol) and triphosgene (0.021 g, 0.07 mmol). The reaction was stirred at rt for 1 h. Octahydrocyclopenta[c]pyrrole (0.033 g, 0.30 mmol) was added. The reaction was stirred for 1 h at rt. The reaction was concentrated in vacuo and purified by reverse phase C₁₈ HPLC eluting with 10-100% acetonitrile/water/0.1% formic acid to afford the title compound as an off-white solid (18 mg, 28% yield). 1H NMR (400 MHz, CHLOROFORM-d) δppm 0.89-1.02 (m, 1H) 1.08-1.22 (m, 2H) 1.24-1.38 (m, 2H) 1.38-1.45 (m, 6H) 1.45-1.57 (m, 6H) 1.58-1.88 (m, 4H) 1.87-2.02 (m, 2H) 2.14 (d, J=7.03 Hz, 2H) 2.20-2.34 (m, 1H) 2.62 (br. s., 3H) 2.66-2.84 (m, 2H) 2.92 (br. s., 1H) 3.18-3.31 (m, 1H) 3.86 (br. s., 5H) 4.06-4.17 (m, 1H) 4.32-4.44 (m, 1H) 4.55-4.73 (m, 2H) 4.90-5.10 (m, 3H) 4.90-5.09 (m, 1H) 5.47-5.60 (m, 1H) 5.53 (br. s., 1H) 5.68-5.89 (m, 1H) 5.69-5.87 (m, 1H) 7.01-7.11 (m, 2H) 7.06 (br. s., 1H) 7.40-7.63 (m, 1H) 7.43-7.61 (m, 1H) 7.95-8.09 (m, 1H) 7.98-8.07 (m, 1H) 10.49-10.60 (m, 1H) 10.49-10.60 (m, 1H). LCMS 902 (M+H).

### Example 25

### (2R,6S,13aS,14aR,16aS)-N-(cyclopropylsulfonyl)-6-({[4-(hydroxymethyl)-1-piperidinyl]carbonyl}amino)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

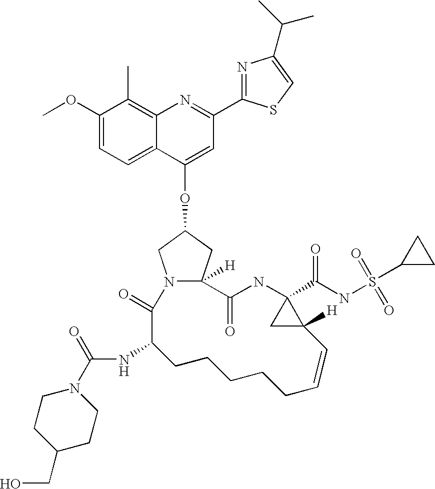

To (2R,6S,13aS,14aR,16aS)-6-amino-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide (free base of intermediate 5) (0.060 g, 0.07 mmol) in dichloroethane (3 mL) was added N,N-diisopropylethylamine (0.019 g, 0.14 mmol) and triphosgene (0.021 g, 0.07 mmol). The reaction was stirred at rt for 1 h. 4-Piperidinylmethanol (0.034 g, 0.30 mmol) was added. The reaction was stirred for 1 h at rt. The reaction was concentrated in vacuo and purified by reverse phase C₁₈ HPLC eluting with 10-100% acetonitrile/water/0.1% formic acid to afford the title compound as an off-white solid (18 mg, 27% yield). 1H NMR (400 MHz, CHLOROFORM-d) δ ppm 0.88-1.00 (m, 1H) 1.06-1.23 (m, 2H) 1.25-1.38 (m, 2H) 1.41 (d, J=7.03 Hz, 6H) 1.45-1.54 (m, 6H) 1.55-1.69 (m, 6H) 1.69-1.76 (m, 4H) 1.94 (br. s., 2H) 2.19-2.28 (m, 1H) 2.55-2.63 (m, 1H) 2.65 (s, 3H) 2.71-2.76 (m, 2H) 2.76-2.83 (m, 1H) 2.88-2.98 (m, 1H) 3.19-3.30 (m, 1H) 3.49 (d, J=5.77 Hz, 2H) 3.79-3.88 (m, 1H) 3.90 (s, 4H) 4.07-4.17 (m, 1H) 4.37-4.45 (m, 1H) 4.63 (br. s., 2H) 4.86-4.95 (m, 1H) 4.97-5.07 (m, 1H) 5.51-5.58 (m, 1H) 5.67-5.81 (m, 1H) 7.05 (s, 1H) 7.07-7.13 (m, 1H) 7.56 (br. s., 1H) 8.00-8.10 (m, 1H) 10.34-10.46 (m, 1H). LCMS 906 (M+H)

### Example 26

### (2R,6S,13aS,14aR,16aS,Z)-N-(cyclopropylsulfonyl)-2-(2-(4-cyclopropylthiazol-2-yl)-7-methoxy-8-methylquinolin-4-yloxy)-6-(3,3-diethylureido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

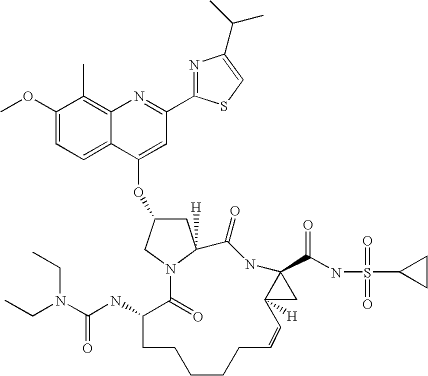

### N-(6-Acetyl-3-methoxy-2-methylphenyl)-4-cyclopropylthiazole-2-carboxamide

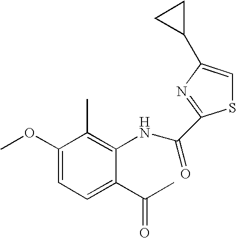

To a stirred solution of 4-cyclopropylthiazole-2-carbonyl chloride (2.8 g, 14.9 mmol) in 100 mL of dioxane was added 1-(2-amino-4-methoxy-3-methylphenyl)ethanone (2.43 g, 13.6 mmol) in 20 mL of dioxane. The reaction was stirred at room temperature for 16 hours. After the mixture was concentrated in vacuo, the residue was purified by ISCO (solid loading, ethyl acetate/hexane 0-40%) to give 3.1 g of title compound as a white solid.

¹H NMR (DMSO-d₆) δ 10.86 (s, 1H), 7.80 (d, 1H), 7.65 (s, 1H), 7.00 (d, 1H), 3.89 (s, 3H), 2.10 (m, 1H), 2.00 (s, 3H), 0.93-0.99 (m, 4H)

### 2-(4-Cyclopropylthiazol-2-yl)-7-methoxy-8-methylquinolin-4-ol

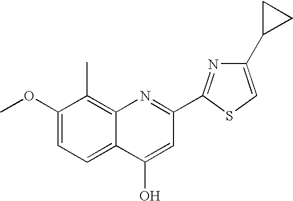

A solution of N-(6-acetyl-3-methoxy-2-methylphenyl)-4-cyclopropylthiazole-2-carboxamide (3.1 g, 9.38 mmol) in 30 mL of pyridine was treated with KOH (1.32 g, 23.5 mmol) at room temperature and the reaction was heated to 110° C. for 3 hours. After the reaction mixture was concentrated to dryness, the residue was diluted with water and neutralized by acetic acid. The precipitate was filtered and dried in vacuo to give 2.84 g of the title compound as a light-yellow solid.

¹H NMR (DMSO-d₆) δ 11.50 (bs, 1H), 7.99 (d, 2H), 7.30-7.45 (m, 3H), 3,93 (s, 3H), 2.48-2.54 (m, 4H), 0.90-1.00 (m, 4H)

### 2-(4-Chloro-7-methoxy-8-methylquinolin-2-yl)-4-cyclopropylthiazole

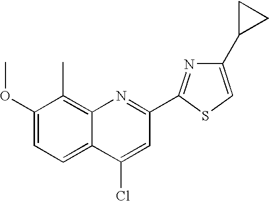

2-(4-cyclopropylthiazol-2-yl)-7-methoxy-8-methylquinolin-4-ol (2.8 g, 9.0 mmol) was mixed with POCl₃ (8.2 mL, 90 mmol) at room temperature and the mixture was heated to 110° C. for 1 h. After the reaction mixture was concentrated to dryness, the residue was diluted with ethyl acetate. The organic layer was washed with saturated NaHCO₃ and brine. The organic layer was dried over anhydrous Na₂SO₄ and concentrated in vacuo to give 2.88 g of the title compound as a yellow solid.

¹H NMR (DMSO-d₆) δ 8.06 (s, 1H), 8.05 (d, 2H), 7.65 (d, 2H), 7.50 (s, 1H), 3.98 (s, 3H), 2.48 (s, 3H), 2.10 (m, 1H), 0.92-0.97 (m, 4H)

### tert-Butyl (2R,6S,13aS,14aR,16aS,Z)-14a-(cyclopropylsulfonylcarbamoyl)-2-(2-(4-cyclopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-6-ylcarbamate

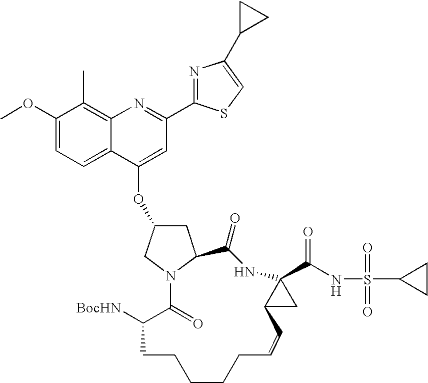

To a stirred solution of 2-(4-chloro-7-methoxy-8-methylquinolin-2-yl)-4-cyclopropylthiazole (166 mg, 0.50 mmol) in 5 mL of DMSO was added t-butyl (2R,6S,13aS,14aR,16aS,Z)-14a-(cyclopropylsulfonylcarbamoyl)-2-hydroxy-5,16-dioxo-1,2,3,5,6,7,8,9,10,11,13a,14,14a,15,16,16a-hexadecahydrocyclopropa[e]pyrrolo[1,2-a][1,4]diazacyclopentadecin-6-ylcarbamate (260 mg, 0.457 mmol) and potassium t-butoxide (256 mg, 2.28 mmol). The reaction was stirred at room temperature for 1 hour. The reaction mixture was diluted with ethyl acetate and washed with water and brine. After the mixture was dried over anhydrous Na₂SO₄ and concentrated in vacuo, the residue was purified by ISCO (solid loading, ethyl acetate/hexane 0-50%) to give 200 mg of title compound as a light-yellow solid.

MS calcd for C₄₃H₅₄N₆O₉S₂: 862.3, MS found (ESI positive): (M+H)⁺=863.3

MS calcd for C₄₃H₅₄N₆O₉S₂: 862.3, MS found (ESI negative): (M−H)⁻=861.3

¹H NMR (CDCl₃) 10.19 (bs, 1H), 7.99 (d, 1H), 7.46 (s, 1H), 7.13 (d, 1H), 6.95 (s, 1H), 6.65 (s, 1H), 5.72 (q, 1H), 5.50 (s, 1H), 4.99 (m, 2H), 4.58-4.69 (m, 2H), 4.29 (m, 1H), 4.04 (d, 1H), 3.95 (s, 3H), 2.90 (m, 1H), 2.72 (m, 2H), 2.67 (s, 3H), 2.58 (m, 1H), 2.29 (q, 1H), 2.16 (m, 1H), 1.70-1.98 (m, 3H), 0.80-1.70 (m, 25H)

### Intermediate 20

### (2R,6S,13aS,14aR,16aS,Z)-6-amino-N-(cyclopropylsulfonyl)-2-(2-(4-cyclopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

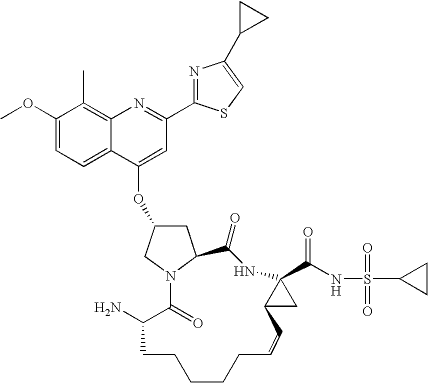

tert-Butyl (2R,6S,13aS,14aR,16aS,Z)-14a-(cyclopropylsulfonylcarbamoyl)-2-(2-(4-cyclopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-6-ylcarbamate (110 mg, 0.127 mmol) was treated with 4.0 M HCl in dioxane (5 mL, 20 mmol) for 2 hours. The reaction was concentrated to afford 97 mg of the title compound as a yellow solid.

MS calcd for C₃₈H₄₆N₆O₇S₂: 762.3, MS found (ESI positive): (M+H)⁺=763.3

MS calcd for C₃₈H₄₆N₆O₇S₂: 762.3, MS found (ESI negative): (M−H)⁻=761.2

¹H NMR (DMSO-d₆) 10.08 (s, 1H), 9.09 (s, 1H), 8.21 (bs, 2H), 8.04 (d, 1H), 7.51 (s, 1H), 7.42 (m, 2H), 5.69 (s, 1H), 5.60 (q, 1H), 5.16 (t, 1H), 4.51 (t, 1H), 3.40-4.40 (m, 5H), 3.95 (s, 3H), 2.91 (m, 1H), 2.67 (m, 1H), 2.57 (s, 3H), 2.19 (m, 2H), 0.80-1.90 (m, 19H)

### (2R,6S,13aS,14aR,16aS,Z)-N-(cyclopropylsulfonyl)-2-(2-(4-cyclopropylthiazol-2-yl)-7-methoxy-8-methylquinolin-4-yloxy)-6-(3,3-diethylureido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

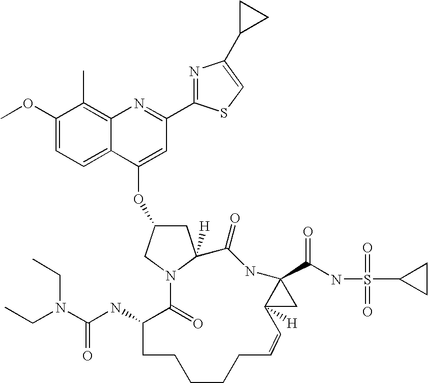

To a stirred solution of (2R,6S,13aS,14aR,16aS,Z)-6-amino-N-(cyclopropylsulfonyl)-2-(2-(4-cyclopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide (intermediate 20) (80 mg, 0.10 mmol) in 5 mL of DCM was added diethylcarbamoyl chloride (57 uL, 0.45 mmol) and TEA (126 uL, 0.9 mmol). The reaction mixture was stirred at room temperature for 72 hours. After the reaction was completed as indicated by LC/MS, the mixture was diluted with DCM and washed with 1N HCl, saturated NaHCO₃ and brine. The organic layer was dried over anhydrous Na₂SO₄. After concentrated in vacuo, the crude residue was purified by prep HPLC (SunFire Prep C18 OBD 5 uM 30×50 mm column) to give 29.1 mg of title compound as a yellow solid.

MS calcd for C₄₃H₆₆N₇O₈S₂: 861.4, MS found (ESI positive): (M+H)⁺=862.3

MS calcd for C₄₃H₅₅N₇O₈S₂: 861.4, MS found (ESI negative): (M−H)⁻=860.3

¹H NMR (CDCl₃) 10.28 (s, 1H), 8.02 (d, 1H), 7.38 (s, 1H), 7.35 (s, 1H), 7.11 (d, 1H), 7.01 (s, 1H), 5.65 (q, 1H), 5.49 (s, 1H), 4.97 (t, 1H), 4.92 (s, 1H), 4.55-4.61 (m, 2H), 4.40 (d, 1H), 4.03 (m, 2H), 3.87 (s, 3H), 3.09 (m, 4H), 2.84 (m, 1H), 2.73 (m, 2H), 2.51 (s, 3H), 2.50 (m, 1H), 2.15 (m, 2H), 1.74-1.94 (m, 3H), 0.98 (t, 6H), 0.80-1.70 (m, 15H)

### Intermediate 21

### 4-Methylene-piperidine-1-carboxylic acid tert-butyl ester

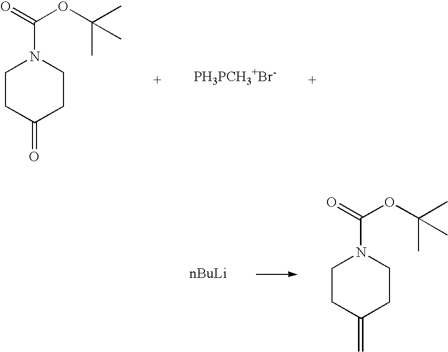

To a solution of methyltriphenylphosphonium bromide (11.12 g, 31 mmol) in THF (42 mL) was added n-BuLi (2.5 M in hexane, 12.5 mL, 31 mmol) at 0° C. N-Boc-4-piperidone (4.15 g, 21 mmol) in THF (21 mL) was added slowly. The reaction was stirred at 0° C. for 2 h before being allowed to warm up to room temperature and stirred at room temperature overnight. The reaction mixture was diluted with dichloromethane (150 mL) and extracted with aqueous HCl (0.5 N, 100 mL), saturated NaHCO₃ (100 mL) and brine (100 mL). The aqueous layers were re-extracted with dichloromethane (2×100 mL). The organic layers were combined and concentrated. Purification by flash column chromatography (5% ethyl acetate in hexane) gave the title compound (3.2 g, 78%) as a white solid.

¹H NMR (300 MHz, CDCl₃) δ 4.75 (2H, s), 3.42 (4H, m), 2.18 (4H, m), 1.46 (9H, s).

### Intermediate 22

### 4-methylenepiperidine hydrochloride

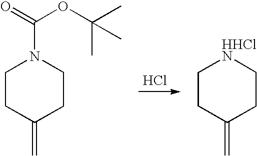

Intermediate 21 (0.86 g, 4.3 mmol) was treated with HCl in dioxane (4N, 60 mL) at room temperature for 30 min. The solvent was removed and the residue was dried under high vacuum to give the title compound as a yellow solid (0.58 g, quant).

### Intermediate 23

### 4-Trifluoromethyl-thiazole-2-carboxylic acid (6-acetyl-2-chloro-3-methoxy-phenyl)-amide

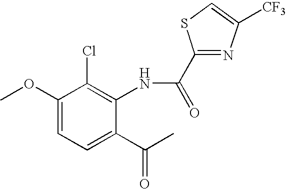

A solution of 4-trifluoromethyl-thiazole-2-carboxylic acid (4.92 g, 25.0 mmol) in DCM was treated with oxalyl chloride (4.46 mL, 50.0 mmol) at room temperature for 1 hour. The mixture was concentrated to afford 4-trifluoromethyl-thiazole-2-carbonyl chloride as an oil (5.37 g, 98%), which was used for next step.

A solution of 1-(2-amino-3-chloro-4-methoxy-phenyl)-ethanone (5.48 g, 22.7 mmol) and pyridine (5.50 mL, 68.1 mmol) in CH₂Cl₂ (50 mL) was treated with 4-trifluoromethyl-thiazole-2-carbonyl chloride at room temperature for 2 h. The mixture was diluted with CH₂Cl₂ and washed with brine. The organic phase was separated, dried (Na₂SO₄), and concentrated. The residue was purified by the flash column chromatography (silica, hexanes/ethyl acetate=1:1) to afford the title compound (5.79 g, 67%).

¹H NMR (300 MHz, CDCl₃) δ 10.38 (s, 1H), 8.03 (s, 1H), 7.74 (d, J=9.0 Hz, 1H), 6.92 (d, J=9.0 Hz, 1H), 4.00 (s, 3H), 2.60 (s, 3H).

### Intermediate 24

### 8-Chloro-7-methoxy-2-(4-trifluoromethyl-thiazol-2-yl)-quinolin-4-ol

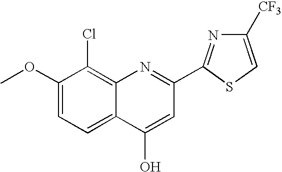

A solution of 4-trifluoromethyl-thiazole-2-carboxylic acid (6-acetyl-2-chloro-3-methoxy-phenyl)-amide (5.78 g, 15.4 mmol) in pyridine (60 mL) was treated with KOH (2.15 g, 38.3 mmol) at room temperature and the reaction was heated to 110° C. for 2 h. The reaction mixture was concentrated to dryness. The residue was diluted with water and neutralized by acetic acid. The precipitate was filtered. The solid was purified by flash column chromatography to afford the title compound as a yellow solid (1.73 g, 31%).

¹H NMR (300 MHz, DMSO-d₆) δ 12.21-12.10 (br, 1H), 8.72 (s, 1H), 8.20 (d, J=9.6 Hz, 1H), 7.63 (d, J=9.6 Hz, 1H), 7.62 (s, 1H), 4.08 (s, 3H).

### Intermediate 25

### 4,8-Dichloro-2-(4-trifluoromethyl-thiazol-2-yl)-7-methoxy-quinoline

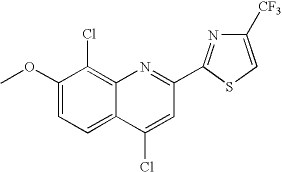

8-Chloro-2-(4-trifluoromethyl-thiazol-2-yl)-7-methoxy-quinolin-4-ol (800 mg, 2.22 mmol) and POCl₃ (2.03 mL, 22.2 mmol) was mixed at room temperature and the mixture was heated to 110° C. for 1 h. The mixture was concentrated to dryness. The residue was dissolved in CH₂Cl₂ and washed with NaHCO₃. The organic phase was separated, dried (Na₂SO₄), and concentrated to afford the title compound (821 mg, 98%).

¹H NMR (300 MHz, DMSO-d₆) δ 8.79 (s, 1H), 8.20 (d, J=9.6 Hz, 1H), 8.19 (s, 1H), 7.84 (d, J=9.3 Hz, 1H), 4.12 (s, 3H).

### Intermediate 26

### t-Butyl (2R,6S,13aS,14aR,16aS,Z)-2-(8-chloro-2-(4-trifluoromethylthiazol-2-yl)-7-methoxyquinolin-4-yloxy)-14a-(cyclopropylsulfonylcarbamoyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-6-ylcarbamate

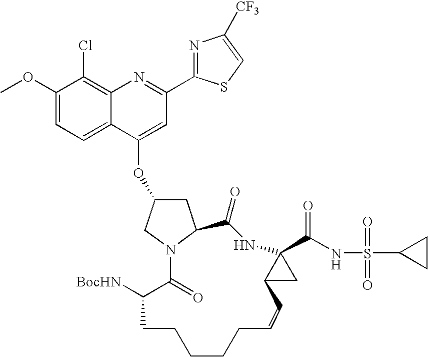

A solution of 4,8-dichloro-2-(4-trifluoromethyl-thiazol-2-yl)-7-methoxy-quinoline (intermediate 25) (146 mg, 0.387 mmol) and t-butyl (2R,6S,13aS,14aR,16aS,Z)-14a-(cyclopropylsulfonylcarbamoyl)-2-hydroxy-5,16-dioxo-1,2,3,5,6,7,8,9,10,11,13a, 14,14a,15,16,16a-hexadecahydrocyclopropa[e]pyrrolo[1,2-a][1,4]diazacyclopentadecin-6-ylcarbamate (200 mg, 0.352 mmol) in DMSO (5 mL) was treated with potassium t-butoxide (237 mg, 2.11 mmol) at room temperature overnight. The reaction was diluted with CH₂Cl₂ and washed with brine. The organic phase was separated, dried (Na₂SO₄), and concentrated. The residue was purified by preparative TLC (silica, hexanes/ethyl acetate=1:2), affording the title compound (190 mg, 59%).

MS calcd for (C₄₀H₄₆ClF₃N₆O₉S₂+H)⁺: 911.2

MS found: (M+H)⁺=911.0

### Intermediate 27

### (2R,6S,13aS,14aR,16aS,Z)-6-amino-2-(8-chloro-2-(4-trifluoromethylthiazol-2-yl)-7-methoxyquinolin-4-yloxy)-N-(cyclopropylsulfonyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide hydrochloride

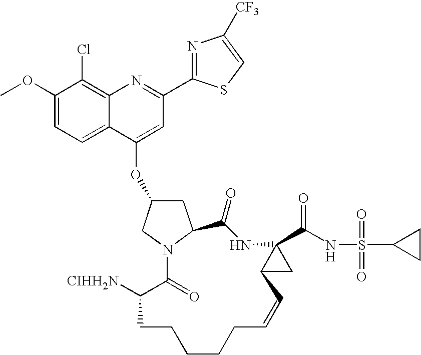

t-Butyl (2R,6S,13aS,14aR,16aS,Z)-2-(8-chloro-2-(4-trifluoromethylthiazol-2-yl)-7-methoxyquinolin-4-yloxy)-14a-(cyclopropylsulfonylcarbamoyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-6-ylcarbamate (164 mg) was treated with HCl (1.0 mL, 4.0 M in dioxane) at room temperature overnight. The reaction was concentrated to afford the title compound as a yellow solid (149 mg, 99%).

MS calcd for (C₃₅H₃₉Cl₂F₃N₆O₇S₂+H−HCl)⁺: 811.2

MS found: (M+H−HCl)⁺=811.0

### Example 27

### (2R,6S,13aS,14aR,16aS,Z)-2-(8-chloro-2(4-cyclopropylthiazol-2-yl)-7-methoxyquinolin-4-yloxy)-N-(cyclopropylsulfonyl)-6-(4-methylenepiperidine-1-carboxamido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

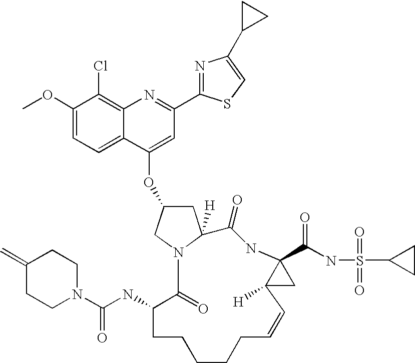

To a mixture of intermediate 19 (100 mg, 0.12 mmol) in dichloromethane (1 mL) and DMF (0.7 mL) was added DIPEA (60 uL, 0.36 mmol) and CDI (30 mg, 0.18 mmol). The mixture was stirred at room temperature overnight. The reaction mixture was added a mixture of intermediate 22 (160 mg, 1.2 mmol) and DIPEA (0.3 mL, 1.8 mmol) in dichloromethane (0.5 mL). The reaction was kept at room temperature overnight. The reaction was diluted with dichloromethane (50 mL) and extracted with aqueous HCl (1N, 50 mL), saturated NaHCO₃ (100 mL) and brine (100 mL). The aqueous phases were re-extracted with dichloromethane (2×25 mL). The organic layers were combined and concentrated. Preparative TLC (dichloromethane:Methanol 10:1) gave the title compound (52 mg, 49%) as yellow solid.

¹H NMR (300 MHz, CDCl₃) δ 10.28 (1H, d), 8.17 (1H, d), 7.56 (1H, s), 7.18 (1H, d), 6.99 (1H, s), 5.70 (1H, s), 5.56 (1H, s), 5.04 (2H, s), 4.76 (2H, s), 4.70 (2H, m), 4.42 (1H, m), 4.05 (3H, m), 3.35 (4H, m), 2.70 (3H, m), 2.18 (6H, m), 1.88 (6H, m), 1.59 (2H, m), 1.48 (6H, m), 1.16 (2H, m), 0.98 (6H, m).

MS calcd for (C44H52ClN7O8S2+H)⁺: 906.3

MS found (electrospray): (M+H)⁺=906.1

### Example 28

### (2R,6S,13aS,14aR,16aS)-2-{[8-chloro-2-(4-cyclopropyl-1,3-thiazol-2-yl)-7-(methyloxy)-4-quinolinyl]oxy}-N-(cyclopropylsulfonyl)-6-[({methyl[2-(methyloxy)ethyl]amino}carbonyl)amino]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

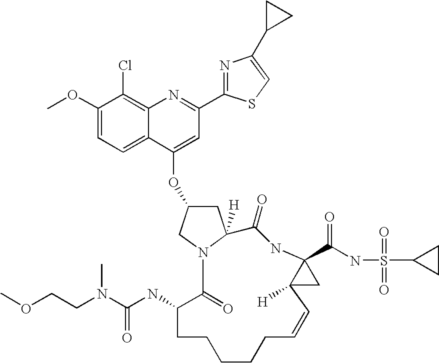

To a solution of intermediate 19 (100 mg, 0.122 mmol) in DCM (1.5 mL) was added N,N-diisopropylethylamine (61 μL, 0.37 mmol), then N,N′-carbonyldiimidazole (30 mg, 0.18 mmol) at room temperature. The solution was stirred overnight. LC-MS showed all the STM was converted to the intermediate. (2-Methoxy-ethyl)-methyl-amine hydrochloride (0.131 mL, 1.22 mmol) was added to the reaction mixture. The resulting solution was stirred at room temperature for 6 hrs. The reaction was diluted with CH₂Cl₂ and washed with 1N HCl, brine. The organic phase was separated, dried (Na₂SO₄), and concentrated. The residue was purified by column chromatography (silica, DCM/MeOH=15:1), affording the title compound (75 mg, 70%) as a white solid.

¹H NMR (CDCl₃) δ 10.42 (bs, 1H), 8.08 (d, 1H), 7.48 (s, 1H), 7.17 (d, 1H), 7.00 (s, 1H), 5.78 (dd, 2H), 5.43 (bs, 1H), 5.04 (t, 1H), 4.58 (m, 2H), 4.28 (m, 1H), 3.99 (s, 3H), 3.42 (m, 2H), 3.38 (s, 3H), 2.84 (s, 3H), 2.61 (m, 3H), 2.22 (m, 2H), 1.92 (m, 2H), 1.62-1.40 (m, 7H), 1.35-1.22 (m, 3H), 1.20-1.02 (m, 3H), 0.98-0.86 (m, 5H).

MS calcd for (C₄₂H₅₂ClN₇O₉S₂+H)⁺: 898

MS found: (M+H)⁺=898

### Example 29

### Cycloprobanesulfonic acid [18-[8-chloro-7-methoxy-2-(4-trifluoromethyl-thiazol-2-yl)-quinolin-4-yloxy]-14-(3,3-diethyl-ureido)-2,15-dioxo-3,16-diaza-tricyclo[14.3.0.04,6]nonadec-7-ene-4-carbonyl]-amide

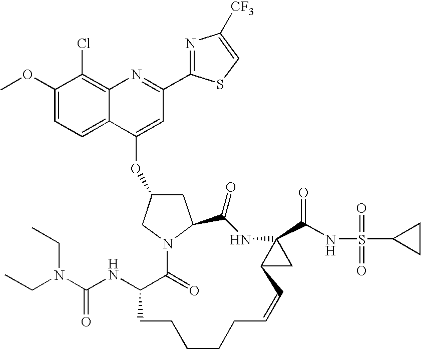

A solution of (2R,6S,13aS,14aR,16aS,Z)-6-amino-2-(8-chloro-2-(4-trifluoromethylthiazol-2-yl)-7-methoxyquinolin-4-yloxy)-N-(cyclopropylsulfonyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide hydrochloride (intermediate 27) (42 mg, 0.050 mmol) in THF was treated with TEA (28 μL, 0.20 mmol) and diethyl carbamyl chloride (10 μL, 0.075 mmol) at room temperature overnight. The reaction was concentrated to dryness. The residue was purified by preparative HPLC to yield the title compound (27 mg, 59%).

¹H NMR (300 MHz, CDCl₃) δ 8.14 (d, J=9.0 Hz, 1H), 8.02 (s, 1H), 7.91 (s, 1H), 7.47 (s, 1H), 7.39 (s, 1H), 7.16 (d, J=9.0 Hz, 1H), 5.75-5.72 (m, 1H), 5.48 (s, 1H), 5.03-4.96 (m, 1H), 4.92-4.90 (m, 1H), 4.67-4.63 (m, 2H), 4.43-4.38 (m, 1H), 4.16-4.10 (m, 1H), 4.01 (s, 3H), 3.83-3.80 (m, 1H), 3.29-3.18 (m, 4H), 2.87-2.82 (m, 1H), 2.76-2.50 (m, 3H), 2.17-2.11 (m, 1H), 1.93-1.83 (m, 2H), 1.58-1.36 (m, 8H), 1.18-1.01 (m, 8H), 0.97-0.86 (m, 2H).

MS calcd for (C₄₀H₄₇ClF₃N₇O₈S₂+H)⁺: 910.3

MS found: (M+H)⁺=910.1

### Example 30

### (2R,6S,13a-5,1-aR,16aS,Z)-N-(cyclopropylsulfonyl)-2-(2-(4-cyclopropylthiazol-2-yl)-7-methoxy-8-methylquinolin-4-yloxy)-5,16-dioxo-6-(pyrrolidine-1-carboxamido)-1,2,3,5,6,7,8,9,10,11,13a,14,14a,15,16,16a-hexadecahydrocyclopropa[e]pyrrolo[1,2-a][1,4]diazacyclopentadecine-14a-carboxamide

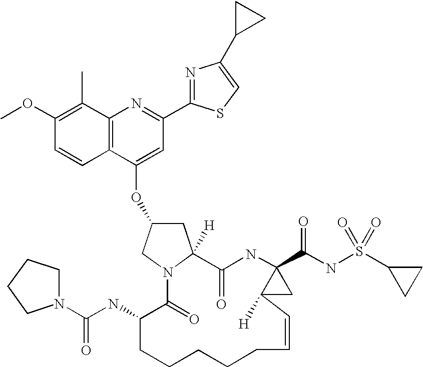

To the mixture of (2R,6S,13aS,14aR,16aS,Z)-6-amino-N-(cyclopropylsulfonyl)-2-(2-(4-cyclopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide hydrochloride (intermediate 20) (240 mg, 0.3 mmol) and 1-pyrrolidinecarbonyl chloride (134 mg, 1 mmol) in 10 ml DCM, added DIPEA (304 mg, 3 mmol) drop wise. Stirred for 2 hours at room temperature. Diluted with 100 ml EtOAc, washed with water (2×20 ml) and brine (20 ml), dried over Na₂SO₄. Concentrated then dissolved the residue in DMSO, purified by preparative HPLC. Obtained 38.5 mg bright yellow powder as title compound.

MS calcd for (C₄₃H₅₃N₇O₈S₂+H)⁺=860.3; MS found (ESI positive): (M+H)⁺=860.3

MS calcd for (C₄₄H₅₃N₇O₃S₂−H)⁻=858.3; MS found (ESI negative): (M−H)⁻=858.3

¹H NMR (400 MHz, CDCl₃) δ 10.33 (s, 1H), 8.08 (d, 1H), 7.46 (s, 2H), 7.20 (d, 1H), 7.09 (s, 1H), 5.72 (dd, 1H), 5.58 (s, 1H), 5.05 (t, 1H), 4.97 (s, 1H), 4.73 (t, 1H), 4.58 (d, 1H), 4.51 (d, 1H), 4.13 (d, 1H), 3.94 (s, 3H), 3.24 (m, 4H), 2.90 (m, 1H), 2.73 (m, 2H), 2.60-2.50 (m, 4H), 2.24 (m, 2H), 2.00-1.75 (m, 7H), 1.74-1.55 (m, 2H), 1.54-1.22 (m, 7H), 1.16 (m, 1H), 1.07 (m, 3H), 1.09-0.90 (m, 3H).

### Example 31

### (2R,6S,13aS,14aR,16aS,Z)-N-(cyclopropylsulfonyl)-2-(2-(4-cyclopropylthiazol-2-yl)-7-methoxy-8-methylquinolin-4-yloxy)-5,16-dioxo-6-(piperidine-1-carboxamido)-1,2,3,5,6,7,8,9,10,11,13a,14,14a,15,16,16a-hexadecahydrocyclopropa[e]pyrrolo[1,2-a][1,4]diazacyclopentadecine-14a-carboxamide

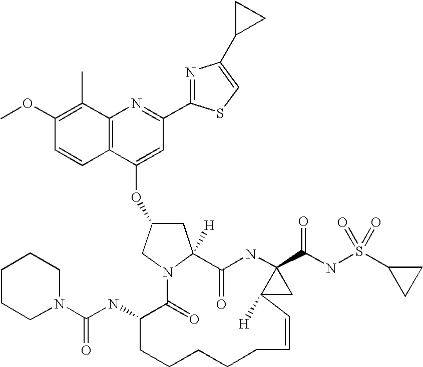

The same procedure as example 30, where 1-pyrrolidinecarbonyl chloride was replaced with 1-piperidinecarbonyl chloride.

MS calcd for (C₄₄H₅₃N₇O₈S₂+H)⁺=874.3; MS found (ESI positive): (M+H)⁺=874.3

MS calcd for (C₄₄H₅₃N₇O₃S₂−H)⁻=872.3; MS found (ESI negative): (M−H)⁻=872.3

¹H NMR (400 MHz, CDCl₃) δ 10.36 (s, 1H), 8.14 (d, 1H), 7.53 (s, 1H), 7.44 (s, 1H), 7.19 (d, 1H), 7.11 (s, 1H), 5.72 (dd, 1H), 5.58 (s, 1H), 5.05 (t, 1H), 4.75-4.60 (m, 2H), 4.40 (d, 1H), 4.08 (m, 1H), 3.94 (s, 3H), 3.38-3.10 (m, 4H), 2.90 (m, 1H), 2.74 (m, 2H), 2.65-2.50 (m, 4H), 2.23 (m, 2H), 2.00-1.75 (m, 3H), 1.74-1.35 (m, 5H), 1.34-1.10 (m, 7H), 1.09-0.80 (m, 9H).

### Example 32

### (2R,65,13aS,14aR,16aS,Z)-6-(azetidine-1-carboxamido)-N-(cyclopropylsulfonyl)-2-(2-(4-cyclopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

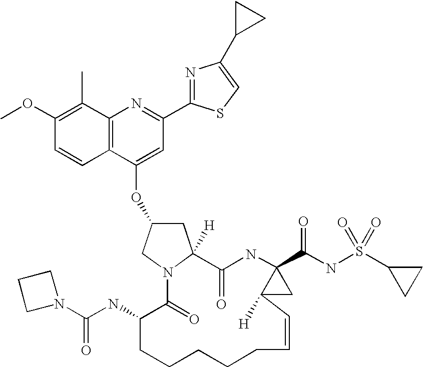

To the mixture of (2R,6S,13aS,14aR,16aS,Z)-6-amino-N-(cyclopropylsulfonyl)-2-(2-(4-cyclopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide hydrochloride (intermediate 20) (240 mg, 0.3 mmol) and 1-pyrrolidinecarbonyl chloride (240 mg, 0.3 mmol) and Di(N-succinimidyl)carbonate (153 mg, 0.6 mmol) in CH₃CN (10 ml), slowly added DIPEA (182 mg, 1.8 mmol). Stirred for 1 hour at room temperature. Then added azetidine hydrochloride (60 mg, 0.6 mmol) followed by DIPEA (121 mg, 1.2 mmol). The result solution was stirred for another 2 hours. Removed solvent, the residue was dissolved in DMSO, then purified by prep HPLC. Obtained 89 mg bright yellow powder as title compound.

MS calcd for (C₄₂H₅₁N₇O₈S₂+H)⁺=846.3; MS found (ESI positive): (M+H)⁺=846.3

MS calcd for (C₄₂H₅₁N₇O₈S₂−H)⁻=844.3; MS found (ESI negative): (M−H)⁻=844.3

¹H NMR (400 MHz, CDCl₃) δ 10.43 (s, 1H), 8.15 (d, 1H), 7.85 (bs, 1H), 7.55 (bs, 1H), 7.30 (d, 1H), 7.20 (s, 1H), 5.75-5.60 (m, 2H), 5.08 (t, 1H), 4.80 (m, 1H), 4.49 (m, 2H), 4.25 (m, 1H), 4.05-3.80 (m, 7H), 2.91 (m, 1H), 2.77 (m, 2H), 2.60-2.45 (m, 4H), 2.27 (m, 4H), 2.0-1.78 (m, 3H), 1.75-1.58 (m, 2H), 1.55-0.90 (m, 15H).

### Example 33

### (2R,6S,13aS,14aR,16aS,Z)-N-(cyclopropylsulfonyl)-2-(2-(4-cyclopropylthiazol-2-yl)-7-methoxy-8-methylquinolin-4-yloxy)-6-(3,3-difluoroazetidine-1-carboxamido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

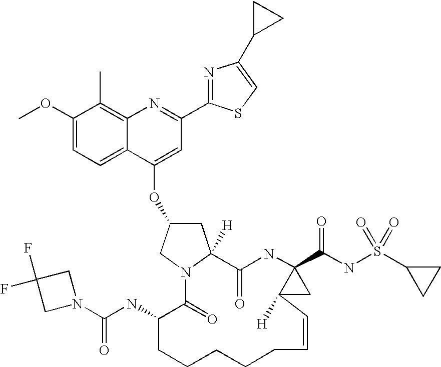

Same procedure as example 32 where azetidine hydrochloride was replaced with 3,3-difluoroazetidine hydrochloride.

MS calcd for (C₄₂H₄₉N₇O₈S₂+H)⁺=882.3; MS found (ESI positive): (M+H)⁺=882.3

MS calcd for (C₄₂H₄₃N₇O₈S₂−H)⁻=880.3; MS found (ESI negative): (M−H)⁻=880.3

¹H NMR (400 MHz, CDCl₃) δ 10.43 (s, 1H), 8.14 (d, 1H), 7.89 (bs, 1H), 7.64 (bs, 1H), 7.33 (d, 1H), 5.75-5.60 (m, 2H), 5.41 (bs, 1H), 5.07 (t, 1H), 4.81 (t, 1H), 4.55 (d, 1H), 4.45 (bs, 1H), 4.35-3.90 (m, 7H), 2.90 (m, 1H), 2.77 (d, 2H), 2.68-2.44 (m, 4H), 2.36-2.20 (m, 2H), 2.05-1.56 (m, 6H), 1.55-0.89 (m, 15H).

### Example 34

### (2R,6S,13a-5,1-aR,16aS,Z)-N-(cyclopropylsulfonyl)-2-(2-(4-cyclopropylthiazol-2-yl)-7-methoxy-8-methylquinolin-4-yloxy)-6-(3,3-dimethylpyrrolidine-1-carboxamido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

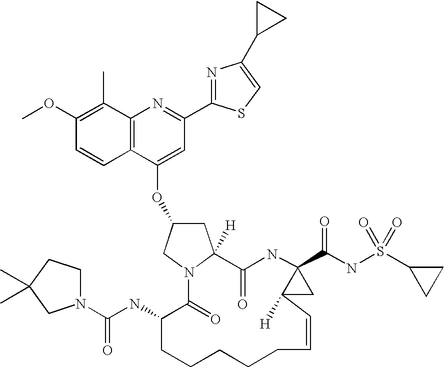

Same procedure as example 33, where azetidine hydrochloride was replaced with 3,3-dimethylpyrrolidine hydrochloride.

MS calcd for (C₄₅H₅₇N₇O₈S₂+H)⁺=888.3; MS found (ESI positive): (M+H)⁺=888.4

MS calcd for (C₄₅H₃₇N₇O₈S₂-H)⁻=886.3; MS found (ESI negative): (M−H)⁻=886.4

¹H NMR (400 MHz, CDCl₃) δ 10.33 (s, 1H), 8.09 (d, 1H), 7.51 (s, 1H), 7.43 (s, 1H), 7.21 (d, 1H), 7.09 (s, 1H), 5.72 (dd, 1H), 5.56 (s, 1H), 5.04 (t, 1H), 4.73 (t, 1H), 4.61 (d, 1H), 4.49 (d, 1H), 4.13 (m, 1H), 3.95 (s, 3H), 2.91 (d, 1H), 2.88 (m, 2H), 2.73 (m, 2H), 2.65-2.50 (m, 4H), 2.30-2.15 (m, 2H), 2.0-1.75 (m, 3H), 1.70-0.85 (m, 27H).

### Example 35

### (2R,6S,13aS,14aR,16aS,Z)-N-(cyclopropylsulfonyl)-6-(3,3-difluoroazetidine-1-carboxamido)-2-(2-(4-isopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

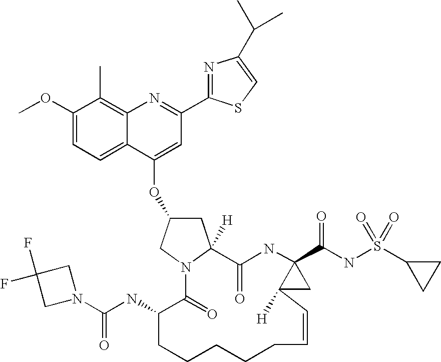

Same procedure as example 33, where (2R,65,13aS,14aR,16aS,Z)-6-amino-N-(cyclopropylsulfonyl)-2-(2-(4-cyclopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide hydrochloride was replaced with (2R,6S,13aS,14aR,16aS,Z)-6-amino-N-(cyclopropylsulfonyl)-2-(2-(4-isopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide hydrochloride (intermediate 5).

MS calcd for (C₄₂H₅₁N₇O₈S₂+H)⁺=884.3; MS found (ESI positive): (M+H)⁺=884.3

MS calcd for (C₄₂H₅₁N₇O₈S₂−H)⁻=882.3; MS found (ESI negative): (M−H)⁻=882.3

¹H NMR (400 MHz, CDCl₃) δ 10.41 (s, 1H), 8.03 (d, 1H), 7.73 (bs, 1H), 7.45 (s, 1H), 7.23 (d, 1H), 6.11 (bs, 1H), 5.70 (dd, 1H), 5.53 (s, 1H), 5.05 (bs, 1H), 5.02 (t, 1H), 4.76 (m, 1H), 4.56 (d, 1H), 4.41 (s, 1H), 4.25-3.90 (m, 7H), 3.29 (m, 1H), 2.88 (m, 1H), 2.80-2.64 (m, 4H), 2.54 (s, 3H), 2.26 (m, 1H), 1.94-1.76 (m, 3H), 1.68-1.53 (m, 2H), 1.54-0.85 (m, 15H).

### Example 36

### (2R,6S,13aS,14aR,16aS,Z)-N-(cyclopropylsulfonyl)-6-(3,3-dimethylpyrrolidine-1-carboxamido)-2-(2-(4-isopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide

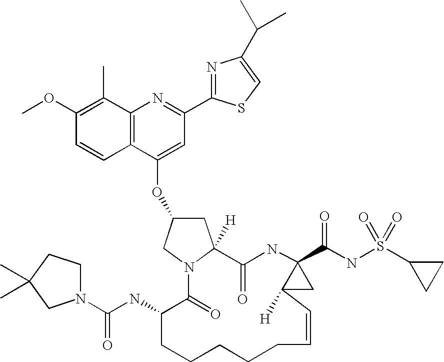

Same procedure as example 35, where 3,3-difluoroazetidine hydrochloride was replaced with 3,3-dimethylpyrrolidine hydrochloride.

MS calcd for (C₄₃H₃₃N₇O₈S₂+H)⁺=890.3; MS found (ESI positive): (M+H)⁺=890.3

MS calcd for (C₄₅H₃₃N₇O₈S₂−H)⁻=888.3; MS found (ESI negative): (M−H)⁻=888.3

¹H NMR (400 MHz, CDCl₃) δ 10.45 (s, 1H), 8.10 (d, 1H), 7.83 (s, 1H), 7.40 (s, 1H), 7.22 (d, 1H), 5.73 (dd, 1H), 5.56 (s, 1H), 5.05 (t, 1H), 4.81 (t, 1H), 4.62 (d, 1H), 4.45 (d, 1H), 4.09 (dd, 1H), 3.89 (s, 3H), 3.40-3.15 (m, 3H), 3.0-2.45 (m, 9H), 2.28 (m, 1H), 1.90 (m, 3H), 1.63 (m, 4H), 1.55-1.20 (m, 14H), 1.24-0.90 (m, 10H).

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

1. A compound of Formula (I): 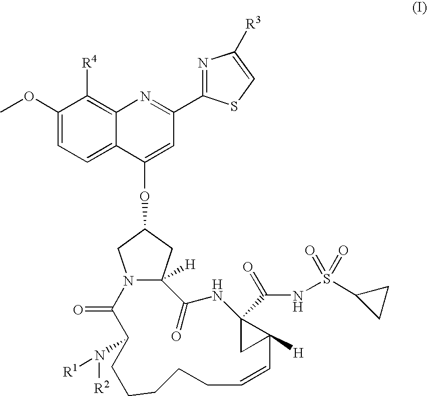
wherein:
R¹ is hydrogen, C₁-C₈ alkyl, haloalkyl, hydroxyalkyl;
R² is C(O)XR^{a}R^{b};
X is N or O;
R^{a} and R^{b} are independently selected from the group consisting of hydrogen, C₁-C₈ alkyl, haloalkyl, hydroxyalkyl, C₁₋₆alkoxy, C₃-C₇ cycloalkyl, heteroaryl, or aryl;
or R^{a} and R^{b} together with the nitrogen to which they are attached form a four to seven membered heterocyclic ring,
R³ and R⁴ are independently selected from the group consisting of C₁-C₈ alkyl, C₁-C₈ hydroxyalkyl, halogen, haloalkyl, Cᵢ₋ₛalkoxy, C₃-C₇ cycloalkyl, heteroaryl, and aryl;
or a pharmaceutically acceptable salt thereof, provided that:
if a) X is N and R^{a} is hydrogen, then R^{b} is not C₁-C₈ alkyl, haloalkyl or C₃-C₇ cycloalkyl; b) X is O, then R^{a} is absent, and R^{b} is not C₁-C₈ alkyl, haloalkyl or C₃-C₇ cycloalkyl.

2. A compound of formula (I) according to claim 1 wherein:
R¹ is hydrogen, C₁-C₈ alkyl, haloalkyl, hydroxyalkyl;
R² is C(O)XR^{a}R^{b};
X is N or O;
R^{a} and R^{b} are independently selected from the group consisting of hydroxyalkyl, C₁₋₆alkoxy, heteroaryl, and aryl, or R^{a} and R^{b} together with the nitrogen to which they are attached form a four to seven membered heterocyclic ring, and wherein if X is O, then R^{a} is absent;
R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
or a pharmaceutically acceptable salt thereof.

3. A compound of formula (I) according to claim 1 wherein:
R¹ is hydrogen, C₁-C₈ alkyl, haloalkyl, hydroxyalkyl;
R² is C(O)XR^{a}R^{b};
X is N or O;
R^{a} and R^{b} together with the nitrogen to which they are attached form a four to seven membered heterocyclic ring, and wherein if X is O, then R^{a} is absent and R^{b} is not hydrogen or C₁₋₆alkoxy;
R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
or a pharmaceutically acceptable salt thereof.

4. A compound of formula (I) according to claim 1 wherein:
R¹ is hydrogen, C₁-C₈ alkyl, haloalkyl, hydroxyalkyl;
R² is C(O)XR^{a}R^{b};
X is N or O;
R^{a} is hydrogen and R^{b} is selected from the group consisting of hydroxyalkyl, heteroaryl, and aryl, or R^{a} and R^{b} together with the nitrogen to which they are attached form a four to seven membered heterocyclic ring;
R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
or a pharmaceutically acceptable salt thereof.

5. A compound of formula (I) according to claim 1 wherein R¹ is hydrogen.

6. A compound of formula (I) according to claim 1 wherein X is O.

7. A compound of formula (I) according claim 1 wherein R^{a} and R^{b} together with the nitrogen atom to which they are attached form a four to seven membered heterocyclic ring.

8. A compound of formula (Ia) 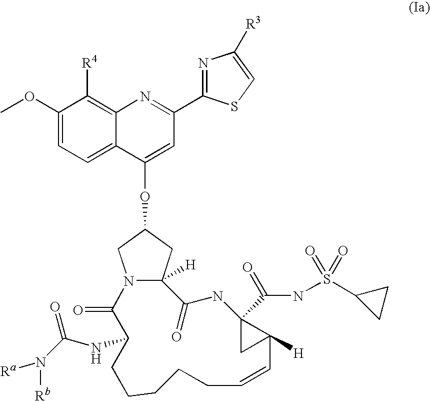
wherein:
R^{a} and R^{b} are independently selected from the group consisting of hydrogen, C₁-C₈ alkyl, haloalkyl, hydroxyalkyl, C₁₋₆alkoxy, C₃-C₇ cycloalkyl, heteroaryl, or aryl;
or R^{a} and R^{b} together with the nitrogen to which they are attached form a four to seven membered heterocyclic ring;
R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
or a pharmaceutically acceptable salt thereof, provided that:
when R^{a} is hydrogen, then R^{b} is not C₁-C₈ alkyl, haloalkyl, or C₃-C₇ cycloalkyl.

9. A compound of formula (Ia) according to claim 8 wherein:
R^{a} and R^{b} are independently selected from the group consisting of C₁-C₈ alkyl, haloalkyl, hydroxyalkyl, C₁₋₆alkoxy, C₃-C₇ cycloalkyl, heteroaryl, or aryl;
or R^{a} and R^{b} together with the nitrogen to which they are attached form a four to seven membered heterocyclic ring;
R³ and R⁴ are independently selected from the group consisting of C₁-C₈ alkyl, C₁-C₈ hydroxyalkyl, halogen, haloalkyl, Cᵢ₋ₑalkoxy, C₃-C₇ cycloalkyl, heteroaryl, and aryl;
or a pharmaceutically acceptable salt thereof.

10. A compound of formula (Ia) according to claim 8 wherein:
R^{a} and R^{b} are independently selected from the group consisting of hydroxyalkyl, C₁₋₆alkoxy, heteroaryl, and aryl, or R^{a} and R^{b} together with the nitrogen to which they are attached form a four to seven membered heterocyclic ring;
R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
or a pharmaceutically acceptable salt thereof.

11. A compound of formula (Ia) according to claim 8 wherein:
R^{a} is hydrogen and R^{b} is selected from the group consisting of hydroxyalkyl, C₁₋₆alkoxy, heteroaryl, and aryl, or R^{a} and R^{b} together with the nitrogen to which they are attached form a four to seven membered heterocyclic ring;
R³ and R⁴ are independently selected from the group consisting of C₁-C₈ alkyl, C₁-C₈ hydroxyalkyl, halogen, haloalkyl, C₁₋₆alkoxy, C₃-C₇ cycloalkyl, heteroaryl, and aryl;
or a pharmaceutically acceptable salt thereof.

12. A compound of formula (I) according to claim 1 wherein R^{a} and R^{b} are both C₁-C₈ alkyl, haloalkyl, hydroxyalkyl, C₃-C₆ cycloalkyl, heteroaryl, and aryl; or a pharmaceutically acceptable salt thereof.

13. A compound of formula (Ia) according to claim 8 wherein R^{a} and R^{b} are both C₁-C₈ alkyl, haloalkyl, hydroxyalkyl, C₃-C₆ cycloalkyl, heteroaryl, and aryl; or a pharmaceutically acceptable salt thereof.

14. A compound of formula (Ia) according to any of claim 8 wherein R^{a} and R^{b} together with the nitrogen atom to which they are attached form a four to seven membered heterocyclic ring.

15. A compound of formula (Ia) 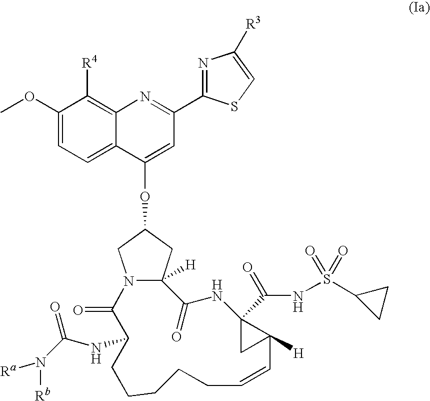
wherein R³ is C₁-C₈ alkyl or C₃-C₆ cycloalkyl; R⁴ is C₁-C₈ alkyl or halogen; R^{a} is hydrogen or C₁-C₈ alkyl, and R^{b} is selected from the group consisting of hydrogen, C₁-C₈ alkyl, C₃-C₇ cycloalkyl, heteroaryl, aryl, cycloalkylalkyl and alkoxyalkyl.

16. A compound of formula (Ia) according to claim 15 wherein R³ is cyclopropyl or isopropyl; R⁴ is C₁-C₈ alkyl; R^{a} is hydrogen or C₁-C₈ alkyl, and R^{b} is selected from the group consisting of hydrogen, C₁-C₈ alkyl, C₃-C₇ cycloalkyl, heteroaryl, aryl, cycloalkylalkyl and alkoxyalkyl.

17. A compound of formula (Ia) 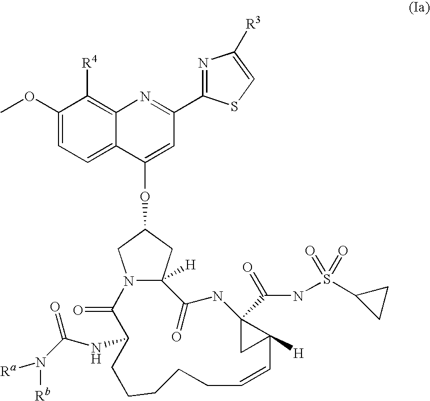
wherein R³ is C₁-C₈ alkyl or C₃-C₆ cycloalkyl; R⁴ is C₁-C₈ alkyl or halogen; and R^{a} and R^{b} together with the nitrogen atom to which they are attached form a four to eight membered heterocyclic ring optionally substituted with one or more substituents selected from the group consisting of C₁-C₈ alkyl, hydroxyalkyl, C₃-C₇ cycloalkyl, alkylidene, hydroxy, halogen, oxo, aryl, heterocyclyl, R^{a}C(O)NH₂ and R^{a}C(O)OH wherein R^{a} is alklylene.

18. A compound of formula (Ia) according to claim 17 wherein R³ is isopropyl or cyclopropyl; and R^{a} and R^{b} together with the nitrogen atom to which they are attached form a four to eight membered heterocyclic ring optionally substituted with one or more substituents selected from the group consisting of C₁-C₈ alkyl, hydroxyalkyl, C₃-C₇ cycloalkyl, alkylidene, hydroxy , halogen, oxo, aryl, heterocyclyl, R^{c}(O)NH₂ and R^{c}(O)OH wherein R^{a} is alklylene.

19. A compound of formula (Ia) according to claim 17 wherein the four to eight membered heterocyclic ring is selected from the group consisting of a morpholinyl, a thiomorpholinyl, a piperidinyl, a piperazinyl, a pyrrolidinyl and an azetidinyl heterocyclic ring.

20. A compound of formula (Ia) according to claim 15 wherein R³ is C₁-C₈ alkyl or C₃-C₆ cycloalkyl; R⁴ is halogen; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

21. A compound of formula (Ia) according to claim 20 wherein R³ is C₁-C₈ alkyl or C₃-C₆ cycloalkyl; R⁴ is chloro; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

22. A compound of formula (Ia) according to claim 15 wherein R³ is cyclopropyl, and R⁴ is C₁-C₈ alkyl or halogen; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

23. A compound of formula (Ia) according to claim 15 wherein R³ is isopropyl, and R⁴ is C₁-C₈ alkyl or halogen; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

24. A compound of formula (Ia) according to claim 15 wherein R³ is C₁-C₈ alkyl or C₃-C₆ cycloalkyl; R⁴ is methyl; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

25. A compound of formula (Ia) according to claim 15 wherein R³ is cyclopropyl or isopropyl, R⁴ is C₁-C₈ alkyl or halogen; R^{a} is hydrogen and R^{b} is hydrogen.

26. A compound of formula (Ia) according to claim 15 wherein R³ is C₁-C₈ alkyl or C₃-C₆ cycloalkyl; R⁴ is methyl; R^{a} is C₁-C₈ alkyl; and R^{b} is selected from the group consisting of C₁-C₈ alkyl, C₃-C₇ cycloalkyl, heteroaryl, aryl, cycloalkylalkyl and alkoxyalkyl.

27. A compound of formula (Ia) according to claim 15 wherein R³ is cyclopropyl or isopropyl; R⁴ is halogen or C₁-C₈ alkyl; R^{a} is methyl, ethyl, propyl or isopropyl, and R^{b} is selected from the group consisting of hydrogen, C₁-C₈ alkyl, C₃-C₇ cycloalkyl, heteroaryl, aryl, cycloalkylalkyl and alkoxyalkyl.

28. A compound of formula (Ia) according to claim 15 wherein R³ is cyclopropyl or isopropyl; R⁴ is halogen or C₁-C₈ alkyl; R^{a} is methyl, ethyl, propyl, or isopropyl, and R^{b} is methyl, ethyl, propyl, and isopropyl.

29. A compound of formula (Ia) according to claim 17 as described above wherein the four to eight membered heterocyclic ring is selected from the group consisting of a morpholinyl, a thiomorpholinyl, a piperidinyl, a piperazinyl, a pyrrolidinyl and an azetidinyl heterocyclic ring.

30. A compound of formula (Ia) according to claim 15 wherein R³ is isopropyl, and R⁴ is C₁-C₈ alkyl or halogen; R^{a} is C₁-C₈ alkyl; and R^{b} is selected from the group consisting of C₁-C₈ alkyl, C₃-C₇ cycloalkyl, heteroaryl, aryl, cycloalkylalkyl and alkoxyalkyl.

31. A compound of formula (Ia) according to claim 15 wherein R³ is isopropyl, and R⁴ is methyl; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

32. A compound of formula (Ia) according to claim 15 wherein R³ is cyclopropyl or isopropyl; R⁴ is methyl; R^{a} is hydrogen or C₁-C₈ alkyl; and R^{b} is selected from the group consisting of hydrogen, C₁-C₈ alkyl, C₃-C₇ cycloalkyl, heteroaryl, aryl, cycloalkylalkyl and alkoxyalkyl.

33. A compound of formula (Ia) according to claim 15 wherein R³ is isopropyl; R⁴ is methyl; R^{a} is methyl or ethyl or propyl or isopropyl, and R^{b} is methyl, ethyl, propyl, or isopropyl.

34. A compound of formula (Ia) according to claim 15 wherein R³ is cyclopropyl or isopropyl, and R⁴ is methyl; R^{a} is C₁-C₈ alkyl, and R^{b} is selected from the group consisting of C₁-C₈ alkyl, C₃-C₇ cycloalkyl, heteroaryl, aryl, cycloalkylalkyl and alkoxyalkyl.

35. A compound of formula (Ia) according to claim 15 wherein R³ is isopropyl, and R⁴ is methyl; R^{a} is C₁-C₈ alkyl, and R^{b} is selected from the group consisting of C₁-C₈ alkyl, C₃-C₇ cycloalkyl, heteroaryl, aryl, cycloalkylalkyl and alkoxyalkyl.

36. A compound of formula (Ia) according to claim 17 wherein R³ is isopropyl, and R⁴ is methyl; R^{a} and R^{b} together with the nitrogen to which they are attached form a four to eight membered heterocyclic ring.

37. A compound of formula (Ia) according to claim 17 wherein R³ is isopropyl; R⁴ is methyl; R^{a} and R^{b} together with the nitrogen to which they are attached form a four to eight membered heterocyclic ring selected from a morpholinyl, a thiomorpholinyl, a hexahydrocyclopenta[c]pyrrol-2(1H)-yl, a piperidinyl, a piperazinyl, a pyrrolidinyl and an azetidinyl heterocyclic ring.

38. A compound of formula (Ia) according to claim 17 wherein R³ is isopropyl, and R⁴ is methyl; R^{a} and R^{b} together with the nitrogen to which they are attached form a four to eight membered heterocyclic ring selected from a piperidinyl, a piperazinyl, a pyrrolidinyl and an azetidinyl heterocyclic ring.

39. A compound of formula (Ia) according to claim 17 wherein R³ is isopropyl or cyclopropyl, and R⁴ is halogen or C₁-C₈ alkyl; R^{a} and R^{b} together with the nitrogen to which they are attached form a four to eight membered heterocyclic ring selected from a piperidinyl, a piperazinyl, a pyrrolidinyl and an azetidinyl heterocyclic ring.

40. A compound of formula (Ia) according to claim 17 wherein R³ is isopropyl or cyclopropyl, and R⁴ is halogen or C₁-C₈ alkyl; R^{a} and R^{b} together with the nitrogen to which they are attached form a piperidinyl ring.

41. A compound of formula (Ia) according to claim 17 wherein R³ is isopropyl, R⁴ is halogen or C₁-C₈ alkyl; R^{a} and R^{b} together with the nitrogen to which they are attached form a piperidinyl heterocyclic ring.

42. A compound of formula (Ia) according to claim 17 wherein R³ is isopropyl or cyclopropyl, and R⁴ is methyl; R^{a} and R^{b} together with the nitrogen to which they are attached form a piperidinyl heterocyclic ring.

43. A compound of formula (Ia) according to claim 17 wherein R³ is isopropyl; R⁴ is halogen or C₁-C₈ alkyl; and R^{a} and R^{b} together with the nitrogen to which they are attached form a four to eight membered heterocyclic ring optionally substituted with one or more substituents selected from the group consisting of C₁-C₈ alkyl, hydroxyalkyl, C₃-C₇ cycloalkyl, alkylidene, hydroxy , halogen, oxo, aryl, heterocyclyl, R^{c}C(O)NH₂ and R^{c}C(O)OH wherein R^{e} is alkylene.

44. A compound of formula (Ia) according to claim 17 wherein R³ is isopropyl or cyclopropyl; R⁴ is methyl; and R^{a} and R^{b} together with the nitrogen to which they are attached form a four to eight membered heterocyclic ring optionally substituted with one or more substituents selected from the group consisting of C₁-C₈ alkyl, hydroxyalkyl, C₃-C₇ cycloalkyl, alkylidene, hydroxy , halogen, oxo, aryl, heterocyclyl, R^{c}C(O)NH₂ and R^{c}C(O)OH wherein R^{c} is alkylene.

45. A compound of formula (Ia) according to claim 17 wherein R³ is isopropyl; R⁴ is methyl; and R^{a} and R^{b} together with the nitrogen to which they are attached form a four to eight membered heterocyclic ring optionally substituted with one or more substituents selected from the group consisting of C₁-C₈ alkyl, hydroxyalkyl, C₃-C₇ cycloalkyl, alkylidene, hydroxy , halogen, oxo, aryl, heterocyclyl, R^{c}C(O)NH₂ and R^{c}C(O)OH wherein R^{c} is alkylene.

46. A compound selected from the group consisting of:
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-{[phenylamino)carbonyl]amino}-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-6-{[(diethylamino)carbonyl]amino}-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-6-[(aminocarbonyl)amino]-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS,Z)-2-(8-chloro-2-(4-cyclopropylthiazol-2-yl)-7-methoxyquinolin-4-yloxy)-N-(cyclopropylsulfonyl)-6-(3,3-diethylureido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
1,1-dimethylethyl ((2R,6S,13aS,14aR,16aS)-14a-{[(cyclopropylsulfonyl)amino]carbonyl}-2-{[8-methyl-2-[4-(1-methylethyl)-1,3-thiazol-2-yl]-7-(methyloxy)-4-quinolinyl]oxy}-5,16-dioxo-1,2,3,5,6,7,8,9,10,11,13a,14,14a,15,16,16a-hexadecahydrocyclopropa[e]pyrrolo[1,2-a][1,4]diazacyclopentadecin-6-yl)carbamate; and
pharmaceutically acceptable salts thereof.

47. A compound selected from the group consisting of:
The present invention features a compound selected from the group consisting of:
(2R,6S,13aS,14aR,16aS)-N-(cyclopropylsulfonyl)-6-({[(2R,6S)-2,6-dimethyl-4-morpholinyl]carbonyl}amino)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-6-{[(1,1-dioxido-4-thiomorpholinyl)carbonyl]amino}-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-6-{[(4-cyclopentyl-1-piperazinyl)carbonyl]amino}-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-6-[(4-morpholinylcarbonyl)amino]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-[(1-pyrrolidinylcarbonyl)amino]-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-[(1-piperidinylcarbonyl)amino]-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-6-[(1-azetidinylcarbonyl)amino]-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-{[(4-phenyl-1-piperidinyl)carbonyl]amino}-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-6-({[4-(1-pyrrolidinyl)-1-piperidinyl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-6-{[(4-hydroxy-1-piperidinyl)carbonyl]amino}-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-6-({[4-(2-amino-2-oxoethyl)-1-piperidinyl]carbonyl}amino)-N-(cyclopropylsulfonyl)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;.
(2R,6S,13aS,14aR,16aS)-N-(cyclopropylsulfonyl)-6-[(hexahydrocyclopenta[c]pyrrol-2(1H)-ylcarbonyl)amino]-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS)-N-(cyclopropylsulfonyl)-6-({[4-(hydroxymethyl)-1-piperidinyl]carbonyl}amino)-2-{[8-methyl-2-[4-(1-methylethyl)-1,3-thiazol-2-yl]-7-(methyloxy)-4-quinolinyl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide;
(2R,6S,13aS,14aR,16aS,Z)-N-(cyclopropylsulfonyl)-2-(2-(4-cyclopropylthiazol-2-yl)-7-methoxy-8-methylquinolin-4-yloxy)-6-(3,3-diethylureido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-2-(8-chloro-2-(4-cyclopropylthiazol-2-yl)-7-methoxyquinolin-4-yloxy)-N-(cyclopropylsulfonyl)-6-(4-methylenepiperidine-1-carboxamido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-N-(cyclopropylsulfonyl)-2-(2-(4-cyclopropylthiazol-2-yl)-7-methoxy-8-methylquinolin-4-yloxy)-5,16-dioxo-6-(pyrrolidine-1-carboxamido)-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-N-(cyclopropylsulfonyl)-2-(2-(4-cyclopropylthiazol-2-yl)-7-methoxy-8-methylquinolin-4-yloxy)-5,16-dioxo-6-(piperidine-1-carboxamido)-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-6-(azetidine-1-carboxamido)-N-(cyclopropylsulfonyl)-2-(2-(4-cyclopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-N-(cyclopropylsulfonyl)-2-(2-(4-cyclopropylthiazol-2-yl)-7-methoxy-8-methylquinolin-4-yloxy)-6-(3,3-difluoroazetidine-1-carboxamido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-N-(cyclopropylsulfonyl)-2-(2-(4-cyclopropylthiazol-2-yl)-7-methoxy-8-methylquinolin-4-yloxy)-6-(3,3-dimethylpyrrolidine-1-carboxamido)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-N-(cyclopropylsulfonyl)-6-(3,3-difluoroazetidine-1-carboxamido)-2-(2-(4-isopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
(2R,6S,13aS,14aR,16aS,Z)-N-(cyclopropylsulfonyl)-6-(3,3-dimethylpyrrolidine-1-carboxamido)-2-(2-(4-isopropylthiazol-2-yl)-7-methoxy-8-methylquinolin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide;
and pharmaceutically acceptable salts thereof.

48. A method of treating or preventing viral infection which comprises administering to a subject in need thereof, an effective amount of a compound as claimed in claim 1.

49. A method as claimed in claim 48 wherein the viral infection is a HCV infection.

50. A compound as claimed in claim 1 for use in medical therapy.

51. A compound as claimed in claim 50 wherein the medical therapy is the treatment of HCV infection.

52. Use of a compound as claimed in claim 1 or a pharmaceutically acceptable salt thereof, in the manufacture of a medicament for the treatment and/or prophylaxis of viral infection.

53. Use as claimed in claim 52 wherein the viral infection is HCV.

54. A pharmaceutical composition comprising a compound as claimed in claim 1 or a pharmaceutically acceptable salt thereof in together with at least one pharmaceutically acceptable diluent or carrier therefor.

55. A pharmaceutical composition as claimed in claim 54 in the form of a tablet, capsule, solution or suspension.

56. A combination comprising a compound as claimed in claim 1, together with at least one other therapeutically active agent.

57. A combination as claimed in claim 56, wherein the other therapeutically active agent is selected from the group consisting of interferon, interferon alfa-2a ,interferon alpha-2b interferon alfacon-1, peginterferon alpha-2b, peginterferon alpha-2a, ribavirin, TMC435350, BI201335, MK-7009, VX950 (telapravir), SCH503034, ITMN191, VCH-759, R7128, BMS-790052, RNAi agents, and cyclophilin inhibitors.

58. A combination as claimed in claim 57, wherein the other therapeutically active agent is selected from the group consisting of interferon, interferon alfa-2a, interferon alpha-2b interferon alfacon-1, peginterferon alpha-2b, peginterferon alpha-2a, and ribavirin


SEQUENCE LISTING (2 sequences)

SEQ ID NO: 1 - protein, 257 aa, homo sapiens
  VARIANT (1)...(257): Xaa = Any Amino Acid
MASMTGGQQMGAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSXXXXXXXXXXXXXXXXXXXXXXXXXXXXXXXXXXXXXXXXXXXXXXXXXXXXXXXXXGSHHHHHH

SEQ ID NO: 2 - protein, 696 aa, homo sapiens
  VARIANT (1)...(696): Xaa = Any Amino Acid
XXXMHHHHHHXAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEEC